(19) United States
(12) Patent Application Publication
(10) Pub. No.: US 2010/0056505 A1
(43) Pub. Date: Mar. 4, 2010

(54) Substituted Pyrazalones

(75) Inventors: Wen-Cherng Lee, Lexington, MA (US); Mary Beth Carter, Lawrence, KS (US); Claudio Chuaqui, Arlington, MA (US); Kevin Guckian, Marlborough, MA (US); Edward Lin, Chesnut Hill, MA (US); Michael J. Choi, San Diego, CA (US); Zhan Deng, Winchester, MA (US); Paula Ann Boriack-Sjodin, Waltham, MA (US); Lihong Sun, Lexington, MA (US)

Correspondence Address: Johnathan P. O'Brien, Ph.D;Honigman Miller Schwartz, 300 East Michigan Ave, Suite 300, KALAMAZOO, MI, 49007, US

(73) Assignee: Biogen Idec MA Inc.

(21) Appl. No.: 12/085,380

(22) PCT Filed: Nov. 21, 2006
(86) PCT No.: PCT/US2006/045095

§ 371 Date: Oct. 15, 2009

Related U.S. Application Data
(60) Provisional application No. 60/738,676, filed on Nov. 21, 2005.

(51) Int. Cl. A61K 31/498; C07D 403/02; C07D 401/14; C07D 409/14; C07D 403/12; C07D 417/14; C07D 413/14; A61K 31/541; A61K 31/5377; A61P 43/00; A61P 35/00; A61P 9/00; A61P 9/12

(52) U.S. Cl. 514/228.2; 544/353; 544/58.1; 544/116; 514/249; 514/234.8

(57) ABSTRACT

The invention is related to compounds of formula (I) as antagonists of the TGFβ family type I receptors, Alk5 and/or AIk 4, compositions and methods of use. The compounds of formula (I) can be employed in the prevention and/or treatment of diseases such as fibrosis (e.g., renal fibrosis, pulmonary fibrosis, and hepatic fibrosis), progressive cancers, or other diseases for which reduction of TGFβ family signaling activity is desirable.

This application claims priority to U.S. Ser. No. 60/738,676, filed Nov. 21, 2005. The entire contents of the aforementioned application are incorporated herein.

### FIELD OF THE INVENTION

The present invention relates to compounds that are useful for modulating Transforming Growth Factor β signaling activity.

### BACKGROUND OF THE INVENTION

TGFβ (Transforming Growth Factor β) is a member of a large family of dimeric polypeptide growth factors that includes, for example, activins, inhibins, bone morphogenetic proteins (BMPs), growth and differentiation factors (GDFs) and mullerian inhibiting substance (MIS). TGFβ exists in three isoforms (TGFβ1, TGFβ2, and TGFβ3) and is present in most cells, along with its receptors. Each isoform is expressed in both a tissue-specific and developmentally regulated fashion. Each TGFβ isoform is synthesized as a precursor protein that is cleaved intracellularly into a C-terminal region (latency associated peptide (LAP)) and an N-terminal region known as mature or active TGFβ. LAP is typically non-covalently associated with mature TGFβ prior to secretion from the cell. The LAP-TGFβ complex cannot bind to the TGFβ receptors and is not biologically active. TGFβ is generally released (and activated) from the complex by a variety of mechanisms including, for example, interaction with thrombospondin-1 or plasmin.

Following activation, TGFβ binds at high affinity to the type II receptor (TGFβRII), a constitutively active serine/threonine kinase. The ligand-bound type II receptor phosphorylates the TGFβ type I receptor (Alk 5) in a glycine/serine rich domain, which allows the type I receptor to recruit and phosphorylate downstream signaling molecules, Smad2 or Smad3. See, e.g., Huse, M. et al., Mol. Cell, 8: 671-682 (2001). Phosphorylated Smad2 or Smad3 can then complex with Smad4, and the entire hetero-Smad complex translocates to the nucleus and regulates transcription of various TGFβ-responsive genes. See, e.g., Massagué, J. Ann. Rev. Biochem. Med., 67: 773 (1998).

Activins are also members of the TGFβ superfamily, which are distinct from TGFβ in that they are homo- or heterodimers of activin βa or βb. Activins signal in a manner similar to TGFβ, that is, by binding to a constitutive serine-threonine receptor kinase, activin type II receptor (ActRIIB), and activating a type I serine-threonine receptor, Alk 4, to phosphorylate Smad2 or Smad3. The consequent formation of a hetero-Smad complex with Smad4 also results in the activin-induced regulation of gene transcription.

Indeed, TGFβ and related factors such as activin regulate a large array of cellular processes, e.g., cell cycle arrest in epithelial and hematopoietic cells, control of mesenchymal cell proliferation and differentiation, inflammatory cell recruitment, immunosuppression, wound healing, and extracellular matrix production. See, e.g., Massagué, J. Ann. Rev Cell. Biol. 6: 594-641 (1990); Roberts, A. B. and Spom M. B., Peptide Growth Factors and Their Receptors, 95: 419-472 Berlin: Springer-Verlag (1990); Roberts, A. B. and Spom M. B., Growth Factors, 8:1-9 (1993); and Alexandrow, M. G., Moses, H. L., Cancer Res., 55: 1452-1457 (1995). Hyperactivity of TGFβ signaling pathway underlies many human disorders (e.g., excess deposition of extracellular matrix, an abnormally high level of inflammatory responses, fibrotic disorders, and progressive cancers). Similarly, activin signaling and over-expression of activin is linked to pathological disorders that involve extracellular matrix accumulation and fibrosis (see, e.g., Matsuse, T. et al., Am. J. Respir. Cell Mol. Biol. 13: 17-24 (1995); Inoue, S. et al., Biochem. Biophys. Res. Comm., 205: 441-448 (1994); Matsuse, T. et al, Am. J. Pathol., 148: 707-713 (1996); De Bleser et al., Hepatology, 26: 905-912 (1997); Pawlowski, J. E., et al., J. Clin. Invest., 100: 639-648 (1997); Sugiyama, M. et al., Gastroenterology, 114: 550-558 (1998); Munz, B. et al., EMBO J., 18: 5205-5215 (1999)), inflammatory responses (see, e.g., Rosendahl, A. et al., Am. J. Repir. Cell Mol. Biol., 25: 60-68 (2001)), cachexia or wasting (see Matzuk, M. M. et al., Proc. Nat. Acad. Sci. USA, 91: 8817-8821 (1994); Coerver, K. A. et al, Mol. Endocrinol. 10: 534-543 (1996); Cipriano, S. C. et al. Endocrinology 141: 2319-27 (2000)), diseases of or pathological responses in the central nervous system (see Logan, A. et al. Eur. J. Neurosci. 11: 2367-2374 (1999); Logan, A. et al. Exp. Neurol. 159: 504-510 (1999); Masliah, E. et al., Neurochem. Int., 39: 393-400 (2001); De Groot, C. J. A. et al, J Neuropathol. Exp. Neurol. 58: 174-187 (1999), John, G. R. et al, Nat Med. 8: 1115-21 (2002)) and hypertension (see Dahly, A. J. et al., Am. J. Physiol. Regul. Integr. Comp. Physiol., 283: R757-67 (2002)). Studies have shown that TGFβ and activin can act synergistically to induce extracellular matrix production (see, e.g., Sugiyama, M. et al., Gastroenterology, 114: 550-558, (1998)). It is therefore desirable to develop modulators (e.g., antagonists) to members of the TGFβ family to prevent and/or treat disorders involving this signaling pathway.

### SUMMARY OF THE INVENTION

The invention is based on the discovery that compounds of formula (I) are potent antagonists of the TGFβ family type I receptors, Alk5 and/or Alk 4. Thus, compounds of formula (I) can be employed in the prevention and/or treatment of diseases such as fibrosis (e.g., renal fibrosis, pulmonary fibrosis, and/or hepatic fibrosis), progressive cancers, or other diseases for which reduction of TGFβ family signaling activity is desirable.

In one aspect, the invention features a compound of formula (I):

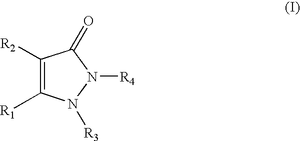

or a pharmaceutically acceptable salt thereof, wherein the variables R₁, R₂, R₃, and R₄ are defined herein.

Compounds of formula (I) exhibit affinity to the TGFβ family type I receptors, Alk5 and/or Alk4, e.g., with IC₅₀ and Kᵢ values of less than about 10 μM (e.g., less than 5.0 μM, 4.5 μM, 4.0 μM, 3.5 μM or 2.5 μM) under conditions as described below in Example 85. Some compounds of formula (I) exhibit IC₅₀ and Kᵢ values of less than 1 μM (e.g., less than 0.90 μM, less than about 0.50 μM, or less than 0.05 μM).

The present invention also features a pharmaceutical composition comprising a compound of formula (I) (or a combination of two or more compounds of formula (I)) and at least one pharmaceutically acceptable carrier. Also included in the present invention is a medicament composition including any of the compounds of formula (I), alone or in a combination, together with a suitable excipient.

The invention also features a method of inhibiting the TGFβ family type I receptors, Alk5 and/or Alk4 (e.g., with an IC₅₀ value of less than 10 μM; such as, less than 1 μM; and for example, less than 5 nM) in a cell, including the step of contacting the cell with an effective amount of one or more compounds of formula (I). Also within the scope of the invention is a method of inhibiting the TGFβ and/or activin signaling pathway in a cell or in a subject (e.g., a mammal such as a human), including the step of contacting the cell with or administering to the subject an effective amount of one or more of the compounds of formula (I).

In another aspect, a method of reducing the accumulation of excess extracellular matrix induced by TGFβ in a subject includes administering to said subject an effective amount of a compound of formula (I).

In another aspect, a method of treating or preventing fibrotic condition in a subject includes administering to said subject an effective amount of a compound of formula (I). The fibrotic condition can be, for example, mesothelioma, acute respiratory distress syndrome (ARDS), atherosclerosis, scleroderma, keloids, glomerulonephritis, diabetic nephropathy, lupus nephritis, hypertension-induced nephropathy, cholangitis, restenosis, ocular scarring, corneal scarring, hepatic fibrosis, biliary fibrosis, liver cirrhosis, cirrhosis due to fatty liver disease (alcoholic and nonalcoholic steatosis), primary sclerosing cholangitis, pulmonary fibrosis (such as bleomycin-induced pulmonary fibrosis, radiation-induced fibrosis, or idiopathic pulmonary fibrosis), renal fibrosis, sarcoidosis, acute lung injury, drug-induced lung injury, spinal cord injury, CNS scarring, systemic lupus erythematosus, Wegener's granulomatosis, cardiac fibrosis, post-infarction cardiac fibrosis, post-surgical fibrosis, connective tissue disease, radiation-induced fibrosis, chemotherapy-induced fibrosis, transplant arteriopathy, fibrosclerosis, fibrotic cancers, fibroids, fibroma, fibroadenomas, fibrosarcomas, wound healing, surgical scarring, chronic obstructive pulmonary disease, alimentary track or gastrointestinal fibrosis. The fibrotic condition can be idiopathic in nature, genetically linked, or induced by radiation.

In another aspect, the compounds of the invention are useful at treating and preventing vascular disease such as intimal thickening or vascular remodeling.

In another aspect, a method of inhibiting growth or metastasis of tumor cells and/or cancers in a subject includes administering to said subject an effective amount of a compound of formula (I).

In another aspect, a method of treating a disease or disorder mediated by an overexpression of TGFβ includes administering to a subject in need of such treatment an effective amount of a compound of formula (I). The disease or disorder can be, for example, demyclination of neurons in multiple sclerosis, Alzheimer's disease, cerebral angiopathy, squamous cell carcinomas, multiple myeloma, melanoma, glioma, glioblastomas, leukemia, sarcomas, leiomyomas, mesothelioma, or carcinomas of the lung, breast, ovary, cervix, liver, biliary tract, gastrointestinal tract, pancreas, prostate, and head and neck.

Other features and advantages of the invention will be apparent from the following detailed description, and from the claims.

### DETAILED DESCRIPTION OF THE INVENTION

### I. Definitions

For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry,” Thomas Sorrell, University Science Books, Sausalito (1999); and “March's Advanced Organic Chemistry,” 5th Ed., Ed.: Smith, M. B. and March, J., John Wiley & Sons, New York (2001).

As used herein the term “aliphatic’ encompasses the terms alkyl, alkenyl, alkynyl, each of which being optionally substituted as set forth below.

As used herein, an “alkyl” group refers to a saturated aliphatic hydrocarbon group containing 1-8 (e.g., 1-6 or 1-4) carbon atoms. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, or 2-ethylhexyl. An alkyl group can be substituted (i.e., optionally substituted) with one or more substituents such as halo, cycloaliphatic, heterocycloaliphatic, aryl, heteroaryl, alkoxy, aroyl, heteroaroyl, cycloaliphaticcarbonyl, (heterocycloaliphatic)carbonyl, nitro, cyano, amino, amido, acyl, sulfonyl, sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroarylalkoxy, or hydroxy. Without limitation, some examples of substituted alkyls include carboxyalkyl (such as HOOC-alkyl, alkoxycarbonylalkyl, and alkylcarbonyloxyalkyl), cyanoalkyl, hydroxyalkyl, alkoxyalkyl, acylalkyl, hydroxyalkyl, aralkyl, (alkoxyaryl)alkyl, (sulfonylamino)alkyl (such as (alkylsulfonylamino)alkyl), aminoalkyl, amidoalkyl, (cycloaliphatic)alkyl, cyanoalkyl, or haloalkyl.

As used herein, an “alkenyl” group refers to an aliphatic carbon group that contains 2-8 (e.g., 2-6 or 2-4) carbon atoms and at least one double bond. Like an alkyl group, an alkenyl group can be straight or branched. Examples of an alkenyl group include, but are not limited to, allyl, isoprenyl, 2-butenyl, and 2-hexenyl. An alkenyl group can be optionally substituted with one or more substituents such as halo, cycloaliphatic, heterocycloaliphatic, aryl, heteroaryl, alkoxy, aroyl, heteroaroyl, (cycloaliphatic)carbonyl, (heterocycloaliphatic)carbonyl, nitro, cyano, amino, amido, acyl, sulfonyl, sulfinyl, sulfanyl, sulfoxy, urea, thiourea, sulfamoyl, sulfamide, oxo, carboxy, carbamoyl, (cycloaliphatic)oxy, (heterocycloaliphatic)oxy, aryloxy, heteroaryloxy, aralkyloxy, (heteroaryl)alkoxy, or hydroxy.

As used herein, an “alkynyl” group refers to an aliphatic carbon group that contains 2-8 (e.g., 2-6 or 2-4) carbon atoms and has at least one triple bond. An alkynyl group can be straight or branched. Examples of an alkynyl group include, but are not limited to, propargyl and butynyl. An alkynyl group can be optionally substituted with one or more substituents such as halo, cycloaliphatic, heterocycloaliphatic, aryl, heteroaryl, alkoxy, aroyl, heteroaroyl, (cycloaliphatic)carbonyl, (heterocycloaliphatic)carbonyl, nitro, cyano, amino, amido, acyl, sulfonyl, sulfinyl, sulfanyl, sulfoxy, urea, thiourea, sulfamoyl, sulfamide, oxo, carboxy, carbamoyl, (cycloaliphatic)oxy, (heterocycloaliphatic)oxy, aryloxy, heteroaryloxy, aralkyloxy, (heteroaryl)alkoxy, or hydroxy.

As used herein, an “amido” encompasses both “aminocarbonyl” and “carbonylamino.” These terms when used alone or in connection with another group refers to an amido group such as N(R^{X})₂—C(O)— or R^{Y}C(O)—N(R^{X})₂— when used terminally and —C(O)—N(R^{X})— or —N(R^{X})—C(O)— when used internally, wherein R^{X} and R^{Y} are defined below. Examples of amido groups include alkylamido (such as alkylcarbonylamino and alkylcarbonylamino), (heterocycloaliphatic)amido, (heteroaralkyl)amido, (heteroaryl)amido, (heterocycloalkyl)alkylamido, arylamido, aralkylamido, (cycloalkyl)alkylamido, and cycloalkylamido.

As used herein, an “amino” group refers to —NR^{X}R^{Y} wherein each of R^{X} and R^{Y} is independently hydrogen, alkyl, cycloaliphatic, (cycloaliphatic)aliphatic, aryl, araliphatic, heterocycloaliphatic, (heterocycloaliphatic)aliphatic, heteroaryl, carboxy, sulfanyl, sulfinyl, sulfonyl, (aliphatic)carbonyl, (cycloaliphatic)carbonyl, ((cycloaliphatic)aliphatic)carbonyl, arylcarbonyl, (araliphatic)carbonyl, (heterocycloaliphatic)carbonyl, ((heterocycloaliphatic)aliphatic)carbonyl, (heteroaryl)carbonyl, or (heteroaraliphatic)carbonyl, each of which being defined herein and being optionally substituted. Examples of amino groups include alkylamino, dialkylamino, and arylamino.

When the term “amino” is not the terminal group (e.g., alkylcarbonylamino), it is represented by —NR^{X}— wherein R^{X} has the same meaning as defined above.

As used herein, an “aryl” group used alone or as part of a larger moiety as in “aralkyl,” “aralkoxy,” or “aryloxyalkyl” refers to monocyclic (e.g., phenyl); bicyclic (e.g., indenyl, naphthalenyl, tetrahydronaphthyl, tetrahydroindenyl); and tricyclic (e.g., fluorenyl tetrahydrofluorenyl, or tetrahydroanthracenyl, anthracenyl). The bicyclic and tricyclic groups include benzofused 2-3 membered carbocyclic rings. For example, a benzofused group includes phenyl fused with two or more C₄₋₈ carbocyclic moieties. An aryl is optionally substituted with one or more substituents including aliphatic (e.g., alkyl, alkenyl, or alkynyl); cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic ring of a benzofused bicyclic or tricyclic aryl); nitro; carboxy; amido; acyl (e.g., aliphaticcarbonyl; (cycloaliphatic)carbonyl; ((cycloaliphatic)aliphatic)carbonyl; (araliphatic)carbonyl; (heterocycloaliphatic)carbonyl; ((heterocycloaliphatic) aliphatic)carbonyl; and (heteroaraliphatic)carbonyl); sulfonyl (e.g., aliphaticsulfonyl and aminosulfonyl); sulfinyl (e.g., aliphaticsulfinyl); sulfanyl (e.g., aliphaticsulfanyl); nitro; cyano; halo; hydroxyl; mercapto; sulfoxy; urea; thiourea; sulfamoyl; sulfamide; and carbamoyl. Alternatively, an aryl can be unsubstituted.

Non-limiting examples of substituted aryls include haloaryl (e.g., mono-, di (such as p,m-dihaloaryl), and (trihalo)aryl); (carboxy)aryl (e.g., (alkoxycarbonyl)aryl, ((aryalkyl)carbonyloxy)aryl, and (alkoxycarbonyl)aryl); (amido)aryl (e.g., (aminocarbonyl)aryl, (((alkylamino)alkyl)aminocarbonyl)aryl, (alkylcarbonyl)aminoaryl, (arylaminocarbonyl)aryl, and (((heteroaryl)amino)carbonyl)aryl); aminoaryl (e.g., ((alkylsulfonyl)amino)aryl and ((dialkyl)amino)aryl); (cyanoalkyl)aryl; (alkoxy)aryl; (sulfamoyl)aryl (e.g., (aminosulfonyl)aryl); (alkylsulfonyl)aryl; (cyano)aryl; (hydroxyalkyl)aryl; ((alkoxy)alkyl)aryl; (hydroxyl)aryl, ((carboxy)alkyl)aryl; (((dialkyl)amino)alkyl)aryl; (nitroalkyl)aryl; (((alkylsulfonyl)amino)alkyl)aryl; ((heterocycloaliphatic)carbonyl)aryl; ((alkylsulfonyl)alkyl)aryl; (cyanoalkyl)aryl; (hydroxyalkyl)aryl; (alkylcarbonyl)aryl; alkylaryl; (trihaloalkyl)aryl; p-amino-m-alkoxycarbonylaryl; p-amino-m-cyanoaryl; p-halo-m-aminoaryl; and (m-(heterocycloaliphatic)-o-(alkyl))aryl.

As used herein, an “araliphatic” such as an “aralkyl” group refers to an aliphatic group (e.g., a C₁₋₄ alkyl group) that is substituted with an aryl group. “Aliphatic,” “alkyl,” and “aryl” are defined herein. An example of an araliphatic such as an aralkyl group is benzyl.

As used herein, a “bicyclic ring system” includes 8-12 (e.g., 9, 10, or 11) membered structures that form two rings, wherein the two rings have at least one atom in common (e.g., 2 atoms in common). Bicyclic ring systems include bicycloaliphatics (e.g., bicycloalkyl or bicycloalkenyl), bicycloheteroaliphatics, bicyclic aryls, and bicyclic heteroaryls.

As used herein, a “cycloaliphatic” group encompasses a “cycloalkyl” group and a “cycloalkenyl” group, each of which being optionally substituted as set forth below.

As used herein, a “cycloalkyl” group refers to a saturated carbocyclic mono- or bicyclic (fused or bridged) ring of 3-10 (e.g., 5-10) carbon atoms. Examples of cycloalkyl groups include cyclopropyl, cyclobutyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, bicyclo[3.3.2.]decyl, bicyclo[2.2.2]octyl, adamantyl, azacycloalkyl, or ((aminocarbonyl)cycloalkyl)cycloalkyl. A “cycloalkenyl” group, as used herein, refers to a non-aromatic carbocyclic ring of 3-10 (e.g., 4-8) carbon atoms having one or more double bonds. Examples of cycloalkenyl groups include cyclopentenyl, 1,4-cyclohexa-di-enyl, cycloheptenyl, cyclooctenyl, hexahydro-indenyl, octahydro-naphthyl, cyclohexenyl, cyclopentenyl, bicyclo[2.2.2]octenyl, and bicyclo[3.3.1]nonenyl.

A cycloalkyl or cycloalkenyl group can be optionally substituted with one or more substituents such as aliphatic (e.g., alkyl, alkenyl, or alkynyl), cycloaliphatic, (cycloaliphatic) aliphatic, heterocycloaliphatic, (heterocycloaliphatic) aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido (e.g., (aliphatic)carbonylamino, (cycloaliphatic)carbonylamino, ((cycloaliphatic)aliphatic)carbonylamino, (aryl)carbonylamino, (araliphatic)carbonylamino, (heterocycloaliphatic)carbonylamino, ((heterocycloaliphatic)aliphatic)carbonylamino, (heteroaryl)carbonylamino, and (heteroaraliphatic)carbonylamino), nitro, carboxy (e.g., HOOC—, alkoxycarbonyl, and alkylcarbonyloxy), acyl (e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, and (heteroaraliphatic)carbonyl), nitro, cyano, halo, hydroxy, mercapto, sulfonyl (e.g., alkylsulfonyl and arylsulfonyl), sulfinyl (e.g., alkylsulfinyl), sulfanyl (e.g., alkylsulfanyl), sulfoxy, urea, thiourea, sulfamoyl, sulfamide, oxo, or carbamoyl.

As used herein, “cyclic moiety” includes cycloaliphatic, heterocycloaliphatic, aryl, or heteroaryl, each of which has been defined previously.

As used herein, the term “heterocycloaliphatic” encompasses a heterocycloalkyl group and a heterocycloalkenyl group, each of which being optionally substituted as set forth below.

As used herein, a “heterocycloalkyl” group refers to a 3-10 membered mono- or bicylic (fused or bridged) (e.g., 5- to 10-membered mono- or bicyclic) saturated ring structure, in which one or more of the ring atoms is a heteroatom (e.g., N, O, S, or combinations thereof). Examples of a heterocycloalkyl group include piperidyl, piperazyl, tetrahydropyranyl, tetrahydrofuryl, 1,4-dioxolanyl, 1,4-dithianyl, 1,3-dioxolanyl, oxazolidyl, isoxazolidyl, morpholinyl, thiomorpholyl, octahydro-benzofuryl, octahydro-chromenyl, octahydro-thiochromenyl, octahydro-indolyl, octahydro-pyrindinyl, decahydro-quinolinyl, octahydro-benzo[b]thiopheneyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.0^{3,7}]nonyl. A monocyclic heterocycloalkyl group can be fused with a phenyl moiety such as tetrahydroisoquinoline. A “heterocycloalkenyl” group, as used herein, refers to a mono- or bicylic (e.g., 5- to 10-membered mono- or bicyclic) non-aromatic ring structure having one or more double bonds, and wherein one or more of the ring atoms is a heteroatom (e.g., N, O, or S). Monocyclic and bicycloheteroaliphatics are numbered according to standard chemical nomenclature.

A heterocycloalkyl or heterocycloalkenyl group can be optionally substituted with one or more substituents such as aliphatic (e.g., alkyl, alkenyl, or alkynyl), cycloaliphatic, (cycloaliphatic) aliphatic, heterocycloaliphatic, (heterocycloaliphatic) aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido (e.g., (aliphatic)carbonylamino, (cycloaliphatic)carbonylamino, ((cycloaliphatic) aliphatic)carbonylamino, (aryl)carbonylamino, (araliphatic)carbonylamino, (heterocycloaliphatic)carbonylamino, ((heterocycloaliphatic) aliphatic)carbonylamino, (heteroaryl)carbonylamino, and (heteroaraliphatic)carbonylamino), nitro, carboxy (e.g., HOOC—, alkoxycarbonyl, and alkylcarbonyloxy), acyl (e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, and (heteroaraliphatic)carbonyl), nitro, cyano, halo, hydroxy, mercapto, sulfonyl (e.g., alkylsulfonyl and arylsulfonyl), sulfinyl (e.g., alkylsulfinyl), sulfanyl (e.g., alkylsulfanyl), sulfoxy, urea, thiourea, sulfamoyl, sulfamide, oxo, or carbamoyl.

A “heteroaryl” group, as used herein, refers to a monocyclic, bicyclic, or tricyclic ring structure having 4 to 15 ring atoms wherein one or more of the ring atoms is a heteroatom (e.g., N, O, S, or combinations thereof) and wherein one or more rings of the bicyclic or tricyclic ring structure is aromatic. A heteroaryl group includes a benzofused ring system having 2 to 3 rings. For example, a benzofused group includes benzo fused with one or two 4 to 8 membered heterocycloaliphatic moieties (e.g., indolizyl, indolyl, isoindolyl, 3H -indolyl, indolinyl, benzo[b]furyl, benzo[b]thiophenyl, quinolinyl, or isoquinolinyl). Some examples of heteroaryl are azetidinyl, pyridyl, 1H-indazolyl, furyl, pyrrolyl, thienyl, thiazolyl, oxazolyl, imidazolyl, tetrazolyl, benzofuryl, isoquinolinyl, benzthiazolyl, xanthene, thioxanthene, phenothiazine, dihydroindole, benzo[1,3]dioxole, benzo[b]furyl, benzo[b]thiophenyl, indazolyl, benzimidazolyl, benzthiazolyl, puryl, cinnolyl, quinolyl, quinazolyl, cinnolyl, phthalazyl, quinazolyl, quinoxalyl, isoquinolyl, 4H-quinolizyl, benzo-1,2,5-thiadiazolyl, or 1,8-naphthyridyl.

Without limitation, monocyclic heteroaryls include furyl, thiophenyl, 214-pyrrolyl, pyrrolyl, oxazolyl, thazolyl, imidazolyl, pyrazolyl, isoxazolyl, isothiazolyl, 1,3,4-thiadiazolyl, 2H-pyranyl, 4-H-pranyl, pyridyl, pyridazyl, pyrimidyl, pyrazolyl, pyrazyl, or 1,3,5-triazyl. Monocyclic heteroaryls are numbered according to standard chemical nomenclature.

Without limitation, bicyclic heteroaryls include indolizyl, indolyl, isoindolyl, 3H-indolyl, indolinyl, benzo[b]furyl, benzo[b]thiophenyl, quinolinyl, isoquinolinyl, indolizyl, isoindolyl, indolyl, benzo[b]furyl, bexo[b]thiophenyl, indazolyl, benzimidazyl, benzthiazolyl, purinyl, 4H-quinolizyl, quinolyl, isoquinolyl, cinnolyl, phthalazyl, quinazolyl, quinoxalyl, 1,8-naphthyridyl, or pteridyl. Bicyclic heteroaryls are numbered according to standard chemical nomenclature.

A heteroaryl is optionally substituted with one or more substituents such as aliphatic (e.g., alkyl, alkenyl, or alkynyl); cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic or heterocyclic ring of a bicyclic or tricyclic heteroaryl); nitro; carboxy; amido; acyl (e.g., aliphaticcarbonyl; (cycloaliphatic)carbonyl; ((cycloaliphatic)aliphatic)carbonyl; (araliphatic)carbonyl; (heterocycloaliphatic)carbonyl; ((heterocycloaliphatic) aliphatic)carbonyl; and (heteroaraliphatic)carbonyl); sulfonyl (e.g., aliphaticsulfonyl and aminosulfonyl); sulfinyl (e.g., aliphaticsulfinyl); sulfanyl (e.g., aliphaticsulfanyl); nitro; cyano; halo; hydroxyl; mercapto; sulfoxy; urea; thiourea; sulfamoyl; sulfamide; or carbamoyl. Alternatively, a heteroaryl can be unsubstituted.

Non-limiting examples of substituted heteroaryls include (halo)heteroaryl (e.g., mono- and di-(halo)heteroaryl); (carboxy)heteroaryl (e.g., (alkoxycarbonyl)heteroaryl); cyanoheteroaryl; aminoheteroaryl (e.g., ((alkylsulfonyl)amino)heteroaryl and ((dialkyl)amino)heteroaryl); (amido)heteroaryl (e.g., aminocarbonylheteroaryl, ((alkylcarbonyl)amino)heteroaryl, ((((alkyl)amino)alkyl)aminocarbonyl)heteroaryl, (((heteroaryl)amino)carbonyl)heteroaryl, ((heterocycloaliphatic)carbonyl)heteroaryl, and ((alkylcarbonyl)amino)heteroaryl); (cyanoalkyl)heteroaryl; (alkoxy)heteroaryl; (sulfamoyl)heteroaryl (e.g., (aminosulfonyl)heteroaryl); (sulfonyl)heteroaryl (e.g., (alkylsulfonyl)heteroaryl); (hydroxyalkyl)heteroaryl; (alkoxyalkyl)heteroaryl; (hydroxyl)heteroaryl; ((carboxy)alkyl)heteroaryl; [((dialkyl)amino)alkyl]heteroaryl; (heterocycloaliphatic)heteroaryl; (cycloaliphatic)heteroaryl; (nitroalkyl)heteroaryl; (((alkylsulfonyl)amino)alkyl)heteroaryl; ((alkylsulfonyl)alkyl)heteroaryl; (cyanoalkyl)heteroaryl; (acyl)heteroaryl (e.g., (alkylcarbonyl)heteroaryl); (alkyl)heteroaryl, and (haloalkyl)heteroaryl (e.g., trihaloalkylheteroaryl).

A “heteroaraliphatic (such as a heteroaralkyl group) as used herein, refers to an aliphatic group (e.g., a C₁₋₄ alkyl group) that is substituted with a heteroaryl group. “Aliphatic,” “alkyl,” and “heteroaryl” have been defined above.

As used herein, an “acyl” group refers to a formyl group or R^{X}—C(O)— (such as -alkyl-C(O)—, also referred to as “alkylcarbonyl”) where R^{X} and “alkyl” have been defined previously. Acetyl and pivaloyl are examples of acyl groups.

As used herein, an “alkoxy” group refers to an alkyl-O— group where “alkyl” has been defined previously.

As used herein, a “carbamoyl” group refers to a group having the structure —O—CO—NR^{X}R^{Y} or —NR^{X}—CO—O—R^{Z} wherein R^{X} and R^{Y} have been defined above and R^{Z} can be aliphatic, aryl, araliphatic, heterocycloaliphatic, heteroaryl, or heteroaraliphatic.

As used herein, a “carboxy” group refers to —COOH, —COOR^{X}, —OC(O)H, —OC(O)R^{X} when used as a terminal group; or —OC(O)— or —C(O)O— when used as an internal group.

As used herein, a “haloaliphatic” group refers to an aliphatic group substituted with 1-3 halogen. For instance, the term haloalkyl includes the group —CF₃.

As used herein, a “mercapto” group refers to —SH.

As used herein, a “sulfo” group refers to —SO₃H or —SO₃R^{X} when used terminally or —S(O)₃— when used internally.

As used herein, a “sulfamide” group refers to the structure —NR^{X}—S(O)₂—NR^{Y}R^{Z} when used terminally and —NR^{X}—S(O)₂—NR^{Y}— when used internally, wherein R^{X}, R^{Y}, and R^{Z} have been defined above.

As used herein, a “sulfamoyl” group refers to the structure —S(O)₂—NR^{X}R^{Y} or —NR^{X}—S(O)₂—R^{Z} when used terminally or —S(O)₂—NR^{X}— or —NR^{X}—S(O)₂— when used internally, wherein R^{X}, R^{Y}, and R^{Z} are defined above.

As used herein a “sulfanyl” group refers to —S—R^{X} when used terminally and —S— when used internally, wherein R^{X} has been defined above. Examples of sulfanyls include alkylsulfanyl.

As used herein a “sulfinyl” group refers to —S(O)—R^{X} when used terminally and —S(O)— when used internally, wherein R^{X} has been defined above.

As used herein, a “sulfonyl” group refers to —S(O)₂—R^{X} when used terminally and —S(O)₂— when used internally, wherein R^{X} has been defined above.

As used herein, a “sulfoxy” group refers to —O—SO—R^{X} or —SO—O—R^{X}, when used terminally and —O—S(O)— or —S(O)—O— when used internally, where R^{X} has been defined above.

As used herein, a “halogen” or “halo” group refers to fluorine, chlorine, bromine or iodine.

As used herein, an “alkoxycarbonyl,” which is encompassed by the term carboxy, used alone or in connection with another group refers to a group such as alkyl-O—C(O)—.

As used herein, an “alkoxyalkyl” refers to an alkyl group such as alkyl-O-alkyl-, wherein alkyl has been defined above.

As used herein, a “carbonyl” refer to —C(O)—.

As used herein, an “oxo” refers to ═O.

As used herein, an “aminoalkyl” refers to the structure (R^{X})₂N-alkyl-.

As used herein, a “cyanoalkyl” refers to the structure (NC)-alkyl-.

As used herein, a “urea” group refers to the structure —NR^{X}—CO—NR^{Y}R^{Z} and a “thiourea” group refers to the structure —NR^{X}—CS—NR^{Y}R^{Z} when used terminally and —NR^{X}—CO—NR^{Y}— or —NR^{X}—CS—NR^{Y}— when used internally, wherein R^{X}, R^{Y}, and R^{Z} have been defined above.

As used herein, a “guanidine” group refers to the structure —N═C(N(R^{X}R^{Y}))N(R^{X}R^{Y}) wherein R^{X} and R^{Y} have been defined above.

As used herein, the term “amidino” group refers to the structure —C═(NR^{X})N(R^{X}R^{Y}) wherein R^{X} and R^{Y} have been defined above.

The terms “terminally” and “internally” refer to the location of a group within a substituent. A group is terminal when the group is present at the end of the substituent not further bonded to the rest of the chemical structure. Carboxyalkyl, i.e., R^{X}O(O)C-alkyl-, is an example of a carboxy group used terminally. A group is internal when the group is present in the middle of a substituent to at the end of the substituent bound to the rest of the chemical structure. Alkylcarboxy (e.g., alkyl-C(O)O— or alkyl-OC(O)—) and alkylearboxyaryl (e.g., alkyl-C(O)O-aryl- or alkyl-O(CO)-aryl-) are examples of carboxy groups used internally.

The phrase “optionally substituted” is used interchangeably with the phrase “substituted or unsubstituted.” As described herein, compounds of the invention can optionally be substituted with one or more substituents, such as are illustrated generally above, or as exemplified by particular classes, subclasses, and species of the invention. As described herein, the variables R₁, R₂, R₃, and R₄, and other variables contained therein formulae I encompass specific groups, such as alkyl and aryl. Unless otherwise noted, each of the specific groups for the variables R₁, R₂, R₃, and R₄, and other variables contained therein can be optionally substituted with one or more substituents described herein. Each substituent of a specific group is further optionally substituted with one to three of halo, cyano, oxoalkoxy, hydroxyl, amino, nitro, aryl, haloalkyl, and alkyl. For instance, an alkyl group can be substituted with alkylsulfanyl and the alkylsulfanyl can be optionally substituted with one to three of halo, cyano, oxoalkoxy, hydroxyl, amino, nitro, aryl, haloalkyl, and alkyl. As an additional example, the cycloalkyl portion of a (cycloalkyl)carbonylamino can be optionally substituted with one to three of halo, cyano, alkoxy, hydroxyl, nitro, haloalkyl, and alkyl. When two alkoxy groups are bound to the same atom or adjacent atoms, the two alkxoy groups can form a ring together with the atom(s) to which they are bound.

In general, the term “substituted,” whether preceded by the term “optionally” or not, refers to the replacement of hydrogen radicals in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a heterocycloalkyl, can be bound to another ring, such as a cycloalkyl, to form a spiro-bicyclic ring system, e.g., both rings share one common atom. As one of ordinary skill in the art will recognize, combinations of substituents envisioned by this invention are those combinations that result in the formation of stable or chemically feasible compounds.

The phrase “stable or chemically feasible,” as used herein, refers to compounds that are not substantially altered when subjected to conditions to allow for their production, detection, and preferably their recovery, purification, and use for one or more of the purposes disclosed herein.

As used herein, an effective amount is defined as the amount required to confer a therapeutic effect on the treated patient, and is typically determined based on age, surface area, weight, and condition of the patient. The interrelationship of dosages for animals and humans (based on milligrams per meter squared of body surface) is described by Freireich et al., Cancer Chemother. Rep., 50: 219 (1966). Body surface area can be approximately determined from height and weight of the patient. See, e.g., Scientific Tables, Geigy Pharmaceuticals, Ardsley, N.Y., 537 (1970).

As used herein, “patient” refers to a mammal, including a human.

An “antagonist,” as used herein, is a molecule that binds to the receptor without activating the receptor. It competes with the endogenous ligand(s) or substrate(s) for binding site(s) on the receptor and, thus inhibits the ability of the receptor to transduce an intracellular signal in response to endogenous ligand binding.

Unless otherwise defined, the number of ring atoms in formula (I) is as follows:

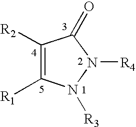

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

### II. Compounds

Compounds of the present invention are useful antagonists of the TGFβ family type I receptors, Alk5 and/or Alk 4.

A. Generic Compounds

Compounds of the present invention include those of formula (I) below:

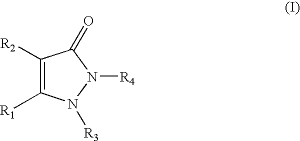

or a pharmaceutically acceptable salt thereof.

R₁ is an optionally substituted 8-12 membered saturated, partially unsaturated, or fully unsaturated bicyclic ring system that has 0-5 heteroatoms independently selected from O, S, or N. R₁ can be optionally substituted with up to 5 substituents selected from (Y—R₅).

Each of R₂ and R₃ is independently hydrogen, halo, an optionally substituted aliphatic, an optionally substituted cycloaliphatic, an optionally substituted aryl, an optionally substituted heteroaryl, an optionally substituted araliphatic, an optionally substituted heterocycloaliphatic, an optionally substituted heteroaryl, or an optionally substituted heteroaraliphatic.

R₄ is hydrogen, halo, aliphatic, cycloaliphatic, (cycloaliphatic)alkyl, aryl, araliphatic, heterocycloaliphatic, (heterocycloaliphatic)alkyl, heteroaryl, or heteroaraliphatic. Each R₄ is optionally substituted with 1 to 3 of (Y—R₅).

R₃ and R₄ together with the nitrogen atoms to which they are attached can also form a 5 to 7 membered heterocyclic ring optionally substituted with 1 to 3 of (Y—R₅)

Each R₅ is independently hydrogen, halo, aliphatic, cycloaliphatic, (cycloaliphatic)alkyl, aryl, amino, cyano, nitro, alkoyx, carbonyl, sulfonyl, araliphatic, heterocycloaliphatic, (heterocycloaliphatic)alkyl, heteroaryl, or heteroaraliphatic.

Each R₅ is optionally substituted with 1 to 3 of halo, aliphatic, amino, cyano, carbonyl, alkoxy, sulfonyl, cycloaliphatic, heterocycloaliphatic, aryl, heteroaryl.

Each Y is independently a bond, —C(O)—, —C(O)—O—, —O—C(O)—, —S(O)ₚ—O—, —O—S(O)ₚ—, —C(O)—N(R^{b})—, —N(R^{b})—C(O)—, —O—C(O)—N(R^{b})—, —N(R^{b})—C(O)—O—, —O—S(O)ₚ—N(R^{b})—, —N(R^{b})—S(O)ₚ—O—, —N(R^{b})—C(O)—N(R^{c})—, —N(R^{b})—S(O)ₚ—N(R^{c})—, —C(O)—N(R^{b})—S(O)ₚ—, —S(O)ₚ—N(R^{b})—C(O)—, —C(O)—N(R^{b})—S(O)ₚ—N(R^{c})—, —C(O)—O—S(O)ₚ—N(R^{b})—, —N(R^{b})—S(O)ₚ—N(R^{c})—C(O)—, —N(R^{b})—S(O)ₚ—O—C(O)—, —S(O)ₚ—N(R^{b})—, —N(R^{b})—S(O)ₚ—, —N(R^{b})—, —S(O)ₚ—, —O—, —S—, or —(C(R^{b})(R^{c}))_{q}—; where each of R^{b} and R^{c} is independently selected from hydrogen, hydroxy, alkyl, alkoxy, amino, aryl, aralkyl, cycloalkyl, heterocycloalkyl, heteroaryl, or heteroaralkyl; and p is 1 or 2, and q is 1-4.

B. Specific Embodiments

i. Substituent R₁

Each R₁ is independently an optionally substituted 8-12 membered saturated, partially unsaturated, or fully unsaturated bicyclic ring system having 0-5 heteroatoms independently selected from O, S, or N, in which, R₁ is optionally substituted with up to 5 substituents selected from (Y—R₅).

In several embodiments, R₁ is an optionally substituted 9 to 111 (e.g., 9, 10, or 11) membered bicyclic ring system. Several examples of R₁ include, but are not limited to bicyclo[4.3.0]-nonane or bicyclo[4.4.0]-decane, each of which is optionally substituted with 1 to 4 substituents.

In several embodiments, R₁ is an optionally substituted 9-11 membered bicyclic aromatic ring system. Examples of R₁ include, but are not limited to indenyl, naphthalenyl, tetrahydronaphthyl, tetrahydroindenyl, azulenyl, and pentahydroazulenyl, each of which is optionally substituted with 1 to 4 substituents.

In several embodiments, R₁ is an optionally substituted bicycloheteroaryl.

In several embodiments, R₁ is an optionally substituted phenyl fused with a 4-8 membered monocyclic heterocycle in which the heterocycle has at least 1 heteroatom. Suitable heteroatoms are N, O, S or combinations thereof.

In several embodiments, R₁ is an optionally substituted indolizinyl, indolyl, isoindolyl, 3H-indolyl, indolinyl, benzo[b]furyl, benzo[b]thiophenyl, quinolinyl, or isoquinolinyl. In several examples, R₁ is optionally substituted with 1-4 substituents independently selected from hydrogen, halo, aliphatic, cycloaliphatic, heterocycloaliphatic, (cycloaliphatic)aliphatic, (heterocycloaliphatic)aliphatic, alkoxy, amino, amido, sulfamoyl, carboxy, sulfonyl, sulfanyl, sulfinyl, aryl, heteroaryl, and aralkyl. In several embodiments, R₁ is unsubstituted.

In several embodiments, R₁ is indolizinyl, and R₁ is bound to the core pyrazalone of formula (I) at any chemically viable position on the bicyclic ring system (e.g., positions 1, 2, 3, 4, 7, or combinations thereof). In several examples of these embodiments, R₁ is optionally substituted at any chemically viable position or positions on the indolozinyl bicyclic ring with one or more substituents selected from (Y—R₅).

In several embodiments, R₁ is indolyl, isoindolyl, 3H-indolyl, indolinyl, benzo[b]furyl, or benzo[b]thiophenyl; and R₁ is bound to the core pyrazalone of formula (I) at any chemically viable position on the bicyclic ring system. In several examples of these embodiments, R₁ is optionally substituted at any chemically viable position or positions on the bicyclic ring system (e.g., positions 1, 2, 3, 4, 7, or combinations thereof) with one or more substituents selected from (Y—R₅).

In several embodiments, R₁ is optionally and independently substituted quinolyl, isoquinolyl, or 4H-quinolizyl; and R₁ is bound to the core pyrazalone of formula I via the 5, 6, 7, or 8 position of the bicycle. R₁ can be optionally and independently substituted at bicycle position 1, 2, 3, 4, or combinations thereof with substituents selected from (Y—R₅).

In several embodiments, R₁ is a phenyl fused with a 4-8 membered monocyclic heterocycle in which the heterocycle has at least 2 heteroatoms each selected from N, O, and S. Examples of R₁ include, but are not limited to optionally substituted 1H-indazolyl, benzimidazolyl, or benzthiazolyl. In several more examples, R₁ is bound to the core pyrazalone of formula I via the 4, 5, 6, or 7 position of the 1H-indazolyl, benzimidazolyl, or benzthiazolyl. In additional examples, R₁ is optionally and independently substituted at bicycle position 1, 2, 3, or combinations thereof with substituents selected from (Y—R₅).

In several embodiments, R₁ is cinnolyl, phthalazyl, quinazolyl, quinoxalyl, or 1,8-naphthyridyl; and R₁ is bound to the core pyrazalone of formula I via the 5, 6, 7, or 8 position of the bicycle. R₁ can be optionally and independently substituted at bicycle position 1, 2, 3, 4, or combinations thereof with substituents selected from (Y—R₅).

In several embodiments, R₁ is a phenyl fused with a 4-8 membered monocyclic heterocycle in which the heterocycle has at least three heteroatoms. Examples of R₁ include, but are not limited to optionally substituted benzo-1,2,5-thiadiazolyl, and R₁ is bound to the core pyrazalone of formula I via the 4, 5, 6, or 7 position of the bicyclic ring system.

In several embodiments, R₁ is quinoxal-1-yl, quinoxal-2-yl, quinoxal-7-yl, or quinoxal-8-yl, cinnol-1-yl, cinnol-2-yl, cinnol-3-yl, cinnol-4-yl, cinnol-5-yl, cinnol-6-yl, cinnol-7-yl, cinnol-8-yl, phthalaz-1-yl, phthalaz-2-yl, phthalaz-3-yl, phthalaz-4-yl, phthalaz-5-yl, phthalaz-6-yl, phthalaz-7-yl, phthalaz-8-yl, quinazol-1-yl, quinazol-2-yl, quinazol-3-yl, quinazol-4-yl, quinazol-5-yl, quinazol-6-yl, quinazol-7-yl, quinazol-8-yl, 1,8-naphthyrid-1-yl, 1,8-naphthyrid-2-yl, 1,8-naphthyrid-3-yl, 1,8-naphthyrid-4-yl, 1,8-naphthyrid-5-yl, 1,8-naphthyrid-6-yl, 1,8-naphthyrid-7-yl, or 1,8-naphthyrid-8-yl, each of which is optionally and independently substituted with one or more substituents selected from (Y—R₅).

Examples of bicyclic heteroaryl R₁ substituents include, but are not limited to

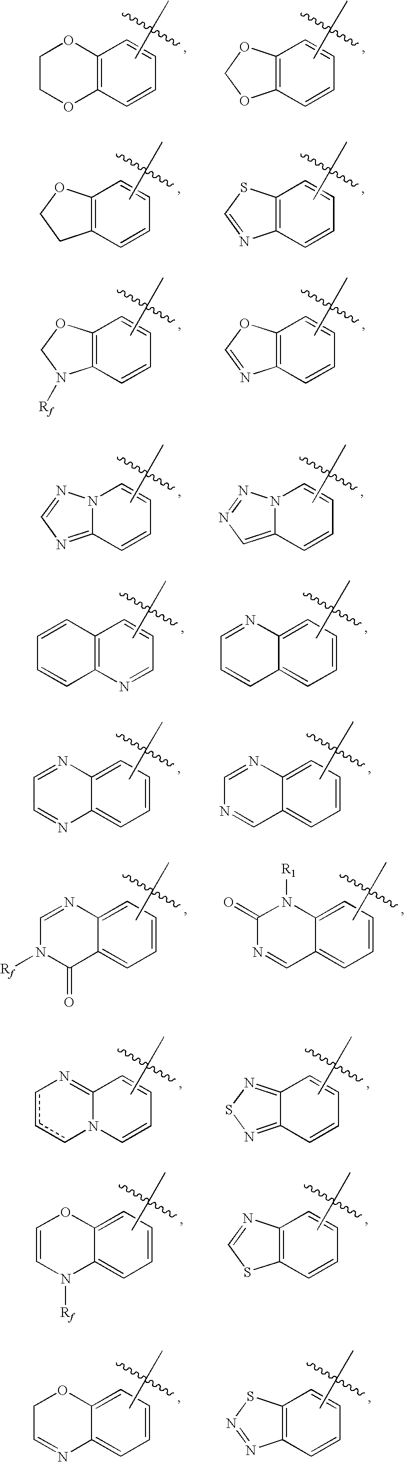

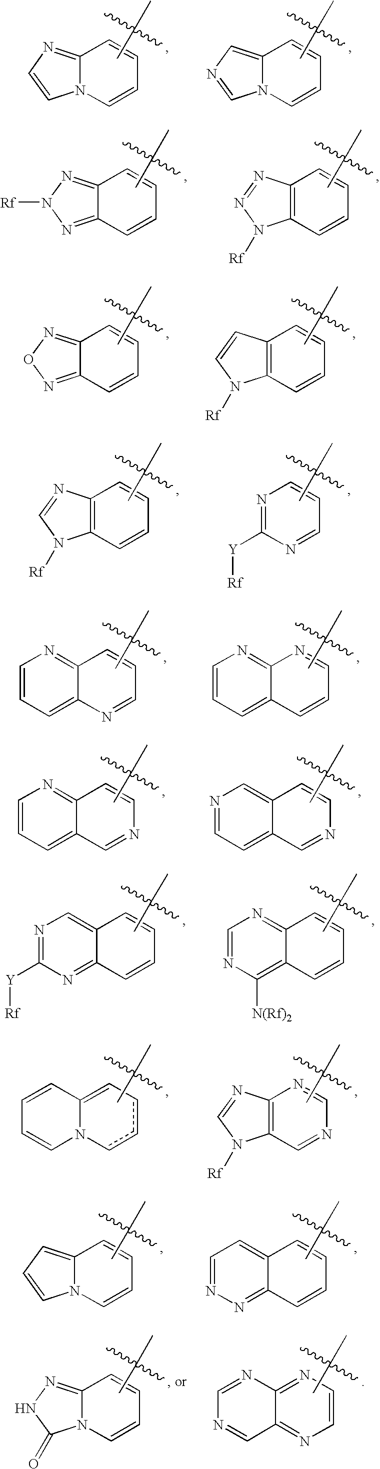

ii. Substituent R₂

In several embodiments, R₂ is hydrogen, halo, aliphatic, cycloaliphatic, heterocycloaliphatic, aryl, heteroaryl, or amino, carbonyl, or sulfonyl. Each R₂ can be optionally substituted.

In several embodiments, R₂ is an optionally substituted 5 to 10 membered ring system. Examples of ring systems include, but are not limited to optionally substituted monocyclic or bicyclic aromatic ring systems.

In several embodiments, R₂ is an optionally substituted phenyl. In several examples, R₂ is substituted with at least 1 substituent at a position meta relative to the point of attachment between R₂ and the pyrazalone ring. In several additional examples, R₂ is substituted with halo, or optionally substituted amido, carboxy, amino, alkoxy, sulfamoyl, sulfonyl, sulfanyl, sulfinyl, or an optionally substituted aliphatic at a position meta relative to the point of attachment between R₂ and the pyrazalone ring.

In several embodiments, R₂ is substituted with at least 1 substituent at a position ortho relative to the point of attachment between R₂ and the pyrazalone ring. In several examples, R₂ is substituted with an amino, cyanoalkyl, alkoxyalkyl, alkoxy, alkyl, cyano, or haloalkyl at a position ortho relative to the point of attachment between R₂ and the pyrazalone ring.

In several embodiments, R₂ is substituted with at least 1 substituent at a position para relative to the point of attachment between R₂ and the pyrazalone ring. In several examples, R₂ is substituted with halo, or optionally substituted cyanoalkyl, morpholylsulfanyl, or haloalkyl at a position para relative to the point of attachment between R₂ and the pyrazalone ring.

In several embodiments, R₂ is a heterocycloaliphatic. Examples of heterocycloaliphatics include, but are not limited to piperidinyl, piperazinyl, 2-pyrazolyl, thiomorpholyl, 2-pyrrolyl, pyrrolidyl, 2-imidizolyl, imidazolyl, imidazolidyl, pyrazolidyl, 1,4-dithiane, 1,3-dioxolanyl, or morpholinyl.

In several embodiments, R₂ is an optionally substituted heteroaryl. Examples of heteroaryls include, but are not limited to monocyclic, bicyclic, or tricyclic ring systems. In additional examples, R₂ is furyl, thiophenyl, 2H-pyrrolyl, pyrrolyl, oxazolyl, thiazolyl, imidazolyl, pyrazolyl, pyridyl, pyridazyl, pyramidyl, pyrazolyl, or pyrazyl; each of which is optionally substituted. R₂ is bound to the core pyrazalone of formula I via the 2, 3, or 4 position of the heteroaryl. R₂ can be optionally substituted at positions 1, 5, 6 or combinations thereof of the heteroaryl. R₂ can be independently substituted with halo, or alkylcarbonyl, carboxy, amido (e.g., (aminoalkylamino)carbonyl), alkoxy, sulfamoly (e.g., alkyl-S(O)₂—NR^{X}—), sulfonyl (e.g., alkyl-S(O)₂—), aminoalkyl, alkoxyalkyl, (aminoalkyl)aminoalkylcarbonyl, alkylcarbonyl, amino, aliphatic, or haloalkyl.

In several embodiments, R₂ is an optionally substituted bicyclic aryl. Suitable aryls are indenyl, naphthalenyl, tetrahydronaphthyl, tetrahydroindenyl, azulenyl, or pentahydroazulenyl.

In several embodiments, R₂ is an optionally substituted bicyclic heteroaryl. R₂ can be an optionally substituted quinolyl, indolyl, 3H-indolyl, isoindolyl, benzo[b]-4H-pyranyl, cinnolyl, quinoxylyl, benzimidazyl, benzo-1,2,5-thiadiazolyl, benzo-1,2,5-oxadiazolyl, or benzthiophenyl. R₂ can bound to the core pyrazalone of formula I via the 2, 3, 5, or 6 position of the bicycle.

In several embodiments, R₂ is optionally substituted with 1 to 3 (e.g., 2) substituents including halo, or optionally substituted carboxy (e.g., alkoxycarbonyl), amido (e.g., (aminoalkyl)aminocarbonyl), alkoxy, sulfamoyl (e.g., alkylS(O)₂NR^{X}—), sulfonyl (e.g., alkylS(O)₂—), aminoalkyl, alkoxyalkyl, alkylcarbonyl, amino, aliphatic, or haloalkyl. In several embodiments, R₂ is unsubstituted.

In several embodiments, R₂ can have no more than 4 substituents independently selected from hydrogen; halo; alkyl (e.g., alkoxyalkyl, carboxyalkyl, hydroxyalkyl, oxoalkyl, aralkyl, (sulfamoyl)alkyl (e.g., alkyl-S(O)₂NR^{X}-alkyl), cyanoalkyl, aminoalkyl, oxoalkyl, alkoxycarbonylalkyl, (cycloalkyl)alkyl heterocycloalkyl, (heterocycloalkyl)alkyl aralkyl, or haloalkyl such as trifluoromethyl); cycloalkyl; (cycloaliphatic)alkyl; aryl; araliphatic; heterocycloaliphatic; (heterocycloaliphatic)alkyl; heteroaryl; heteroaraliphatic; alkenyl, alkynyl, cycloalkyl (e.g., cyclopropyl, cyclobutyl, cyclopentyl, and cyclohexyl); heterocylcoalkyl (e.g., thiomorpholyl, piperazinyl, 1,3,5-trithianyl, morpholinyl, pyrrolyl, 1,3-dioxolanyt, pyrazolidyl, or piperidinyl); aryl; heteroaryl; alkoxy; cycloalkyloxy; heterocycloalkyloxy; aryloxy; heteroaryloxy; aralkyloxy; heteroaralkyloxy; aroyl; heteroaroyl; amido (e.g, alkylcarbonylamino, arylcarbonylamino, cycloalkylcarbonylamino, cycloalkylcarbonylamino, arylcarbonylamino, heteroarylcarbonylamino, (heterocycloalkyl)carbonylamino, arylaminocarbonyl, thiazoleaminocarbonyl, alkylaminoalkylaminocarbonyl, (cycloalkyl)alkylcarbonylamino, (heterocycloalkyl)alkylcarbonylamino), sulfamoyl; nitro; carboxy; alkylcarbonyl; thiomorpholinecarbonyl; cyano; hydroxyl; acyl; mercapto; sulfonyl (e.g., aminosulfonyl, alkylsulfonyl, morpholinesulfonyl, or arylsulfonyl); sulfinyl (e.g., alkylsulfinyl); sulfanyl (e.g., alkylsulfanyl); sulfoxy; urea; thiourea; sulfamoyl; sulfamide; oxo; or carbamoyl.

In several embodiments, R₂ is hydrogen, halo, aliphatic (e.g., alkyl, alkenyl, or alkynyl), aryl, 5-7 membered cycloaliphatic, 5-7 membered heterocycloaliphatic (e.g., piperidinyl, piperazinyl, tetrahydropyranyl, tetrahydrofuryl, dioxolanyl, oxazolidinyl, isoxazolidinyl, morpholinyl, octahydro-benzofuryl, octahydro-chromenyl, octahydro-thiochromenyl, octahydro-indolyl, octahydro-pyrindinyl, decahydro-quinolinyl, octahydro-benzo[b]thiopheneyl, 2-oxa-bicyclo[2.2.2]octyl, 1-aza-bicyclo[2.2.2]octyl, 3-aza-bicyclo[3.2.1]octyl, or 2,6-dioxa-tricyclo[3.3.1.0^{3,7}]nonyl), aryl, or heteroaryl. R₂ can be an aryl such as optionally substituted phenyl, naphthalenyl, azulenyl, fluoroenyl, or antracenyl.

In several embodiments, R₂ is a phenyl with least 1 substituent at a position meta or ortho relative to the point of attachment between R₂ and the pyrazalone ring. R₂ is meta or ortho substituted with halo, alkoxycarbonyl, dialkylaminocarbonyl, amino, cyano, alkylcarbonylamino, cyanoalkyl, alkoxy, sulfamoyl (e.g., alkylsulfonylamino), alkylsulfonyl, aminoalkyl, alkyl, hydroxyalkyl, alkoxyalkyl, hydroxyl, carboxyalkyl, dialkylaminoalkyl, sulfonylheterocycloalkyl, heterocycloarylamido, alkylsulfonylaminoalkyl, heterocycloalkylcarbonyl, dialkylaminoalkylamido, heterocycloalkylcarbonyl, oxoheterocycloalkylcarbonyl, alkylcarbonyl, amido, dialkylamino, or haloalkyl.

In several embodiments, R₂ is a phenyl that is substituted at a position para relative to the point of attachment between R₂ and the pyrazalone ring with halo, aminosulfonyl, alkylsulfonylalkyl, hydroxyl, alkylcarbonylamino, amino, alkyl, or alkoxy.

In alternative embodiments, R₂ is one selected from the group consisting of:

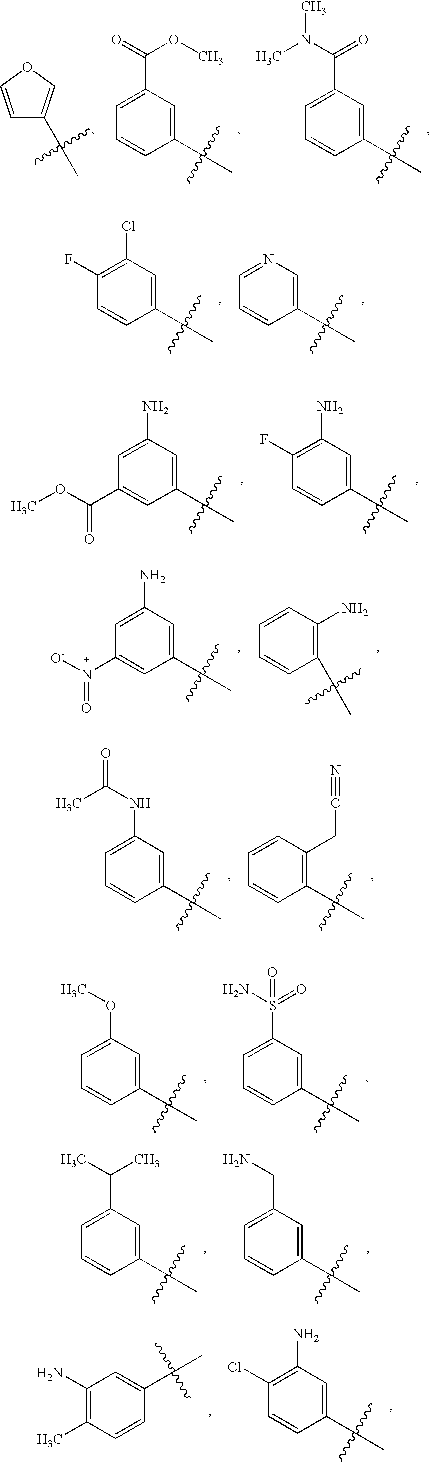

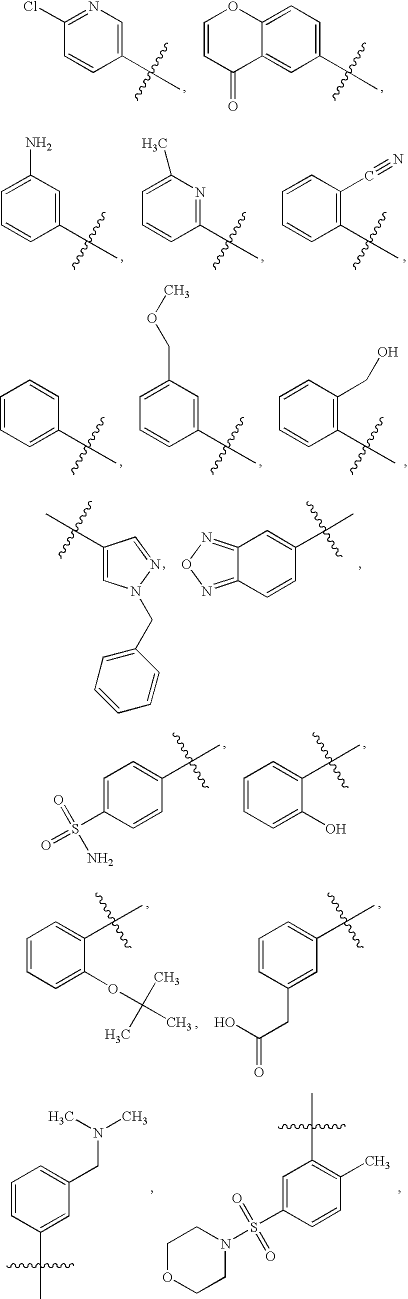

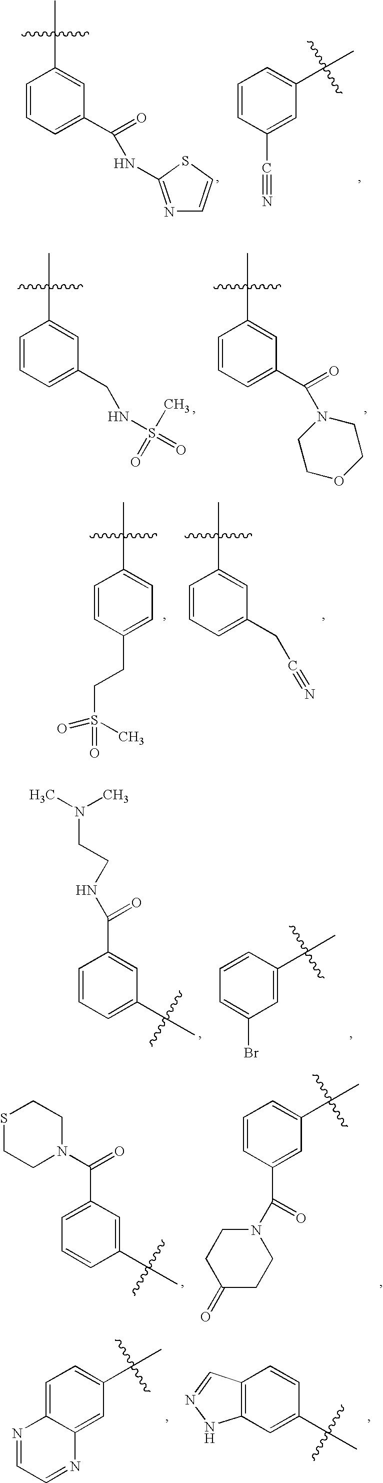

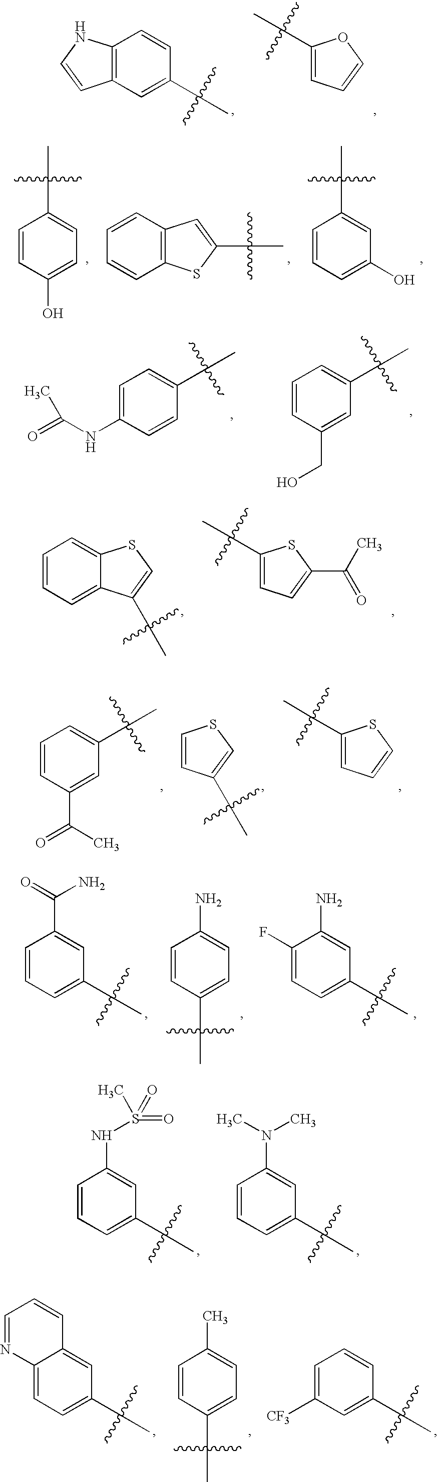

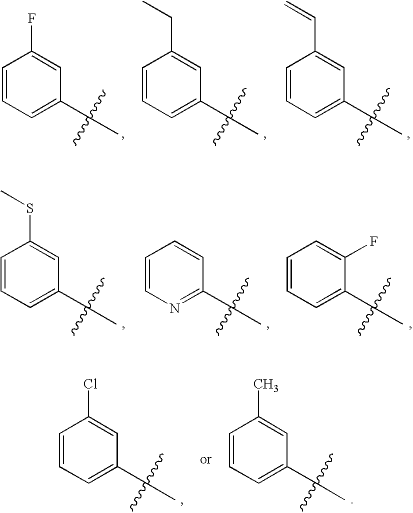

iii. Substituent R₃

R₃ can be hydrogen, halo, an optionally substituted aliphatic, an optionally substituted cycloaliphatic, an optionally substituted heterocycloaliphatic, an optionally substituted aryl, an optionally substituted heteroaryl, amino, amido, sulfamoyl, or sulfonyl.

In several embodiments, R₃ is an optionally substituted 5 to 10 membered ring system. The ring system can be an optionally substituted monocyclic or bicyclic aromatic ring system.

In several embodiments, R₃ is an optionally substituted aryl. A suitable aryl can be an optionally substituted phenyl.

In several embodiments, R₃ is substituted with at least 1 substituent at a position meta relative to the point of attachment between R₃ and the pyrazalone ring. R₃ can be independently meta substituted with halo, alkycarbonyl, carboxy, amino, amido, alkoxy, sulfonyl, sulfanyl, sulfinyl, or aliphatic.

In several embodiments, R₃ is substituted with at least 1 substituent at a position ortho relative to the point of attachment between R₃ and the pyrazalone ring. R₃ is ortho substituted with an amino, cyanoalkyl, alkoxyalkyl, alkoxy, alkyl, cyano, or haloalkyl.

In several embodiments, R₃ is substituted with at least 1 substituent at a position para relative to the point of attachment between R₃ and the pyrazalone ring. R₃ is para substituted with halo, cyanoalkyl, morpholinylsulfonyl, or haloalkyl.

In several embodiments, R₃ is a heterocycloaliphatic. Suitable heterocycloaliphatics are piperidinyl, piperazinyl, 2-pyrazolyl, thiomorpholyl, 2-pyrrolyl, pyrrolidyl, 2-imidizolyl, imidazolyl, imidazolidyl, pyrazolidyl, 1,4-dithiane, 1,3-dioxolanyl, or morpholinyl.

In several embodiments, R₃ is an optionally substituted heteroaryl. Suitable heteroaryls are monocyclic, bicyclic, or tricyclic structures. In several embodiments, R₃ is a furyl, thiophenyl, 2H-pyrrolyl, pyrrolyl, oxazolyl, thiazolyl, imidazolyl, pyrazolyl, pyridyl, pyridazyl, pyramidyl, pyrazolyl, or pyrazyl; each of which is optionally substituted. In several embodiments, R₃ is bound to the core pyrazalone of formula I via the 2, 3, or 4 position of the heteroaryl In several embodiments, R₃ is optionally substituted at ring positions 1, 5, 6 or combinations thereof on the heteroaryl. R₃ is substituted with halo, carboxy, alkylcarbonyl, amido (e.g., (aminoalkylamino)carbonyl), alkoxy, sulfamoyl (alkylsulfonylamino), alkylsulfonyl, aminoalkyl, alkoxyalkyl, alkylcarbonyl, amino, aliphatic, or haloalkyl.

In several embodiments, R₃ is an optionally substituted bicyclic aryl. Suitable aryls are indenyl, naphthalenyl, tetrahydronaphthyl, tetrahydroindenyl, azulenyl, or the like.

In several embodiments, R₃ is an optionally substituted bicyclic heteroaryl. R₃ can be an optionally substituted quinolyl, indolyl, 3H-indolyl, isoindolyl, benzo[b]-4H-pyranyl, cinnolyl, quinoxylyl, benzimidazyl, benzo-1,2,5-thiadiazolyl, benzo-1,2,5-oxadiazolyl, or benzthiophenyl. In several embodiments, R₃ is bound to the core pyrazalone of formula I via the 2, 3, 5, or 6 position of the bicycle.

In several embodiments, R₃ is bound to the core pyrazalone of formula I via the 5, 6, 7 or 8 position of the bicycle. In several examples, R₃ is optionally substituted with 1 to 3 (e.g., 2) substituents including halo, carboxy, alkylcarbonyl, amido (e.g., (aminoalkylamino)carbonyl), alkoxy, sulfamoyl (e.g., alkylsulfonylamino), alkylsulfonyl, aminoalkyl, alkoxyalkyl, alkylcarbonyl, amino, aliphatic, or haloalkyl. In several embodiments, R₃ is unsubstituted.

In several embodiments, R₃ has no more than 7 substituents independently selected from hydrogen; halo; alkyl (e.g., methyl, ethyl, propyl, isopropyl, butyl, isobutyl, sec-butyl, tert-butyl, n-pentyl, n-heptyl, or 2-ethylhexyl); cycloalkyl; (cycloaliphatic)alkyl; aryl; araliphatic; heterocycloaliphatic; (heterocycloaliphatic)alkyl; heteroaryl; aryl; aralkyl; heteroaraliphatic; alkenyl; alkynyl; cycloalkyl (e.g., cyclopropyl, cyclobutyl, cyclopentyl, or cyclohexyl); heterocylcoalkyl (e.g., thiomorpholyl, piperazinyl, 1,3,5-trithianyl, morpholinyl, pyrrolyl, 1,3-dioxolanyl, pyrazolidyl, or piperidinyl); heteroaryl; alkoxy; cycloalkyloxy; heterocycloalkyloxy; aryloxy; heteroaryloxy; aralkyloxy; heteroaralkyloxy; aroyl; heteroaroyl; amino, amido (e.g, alkylcarbonylamino, alkylsulfonylamino, arylcarbonylamino, cycloalkylcarbonylamino, arylcarbonylamino, heteroarylcarbonylamino, (heterocycloalkyl)carbonylamino, (cycloalkyl)alkylcarbonylamino, aminoalkylaminocarbonyl, arylaminocarbonyl, thiazoleaminocarbonyl, or (heterocycloalkyl)alkylcarbonylamino); nitro; carboxy (e.g., alkoxycarbonyl); alkylcarbonyl; thiomorpholinecarbonyl; alkylcarbonyloxy; hydroxyl; acyl; mercapto; sulfonyl (e.g., aminosulfonyl, alkylsulfonyl, morpholinesulfonyl, or arylsulfonyl); sulfinyl (e.g., alkylsulfinyl); sulfanyl (e.g., alkylsulfanyl); sulfoxy; urea; thiourea; sulfamoyl; sulfamide; oxo; or carbamoyl.

In several embodiments, R₃ is hydrogen, halo, aliphatic (e.g., alkyl, alkenyl, or alkynyl), aryl, 5-7 membered cycloaliphatic, 5-7 membered heterocycloaliphatic (e.g., piperidinyl, piperazinyl, tetrahydropyranyl, tetrahydrofuryl, dioxolanyl, oxazolidinyl, isoxazolidinyl, morpholinyl, octahydro-benzofuryl, octahydro-chromenyl, octahydro-thiochromenyl, octahydro-indolyl, octahydro-pyrindinyl, decahydro-quinolinyl, octahydro-benzo[b]thiopheneyl, 2-oxa-bicyclo[2.2.2]octyl, 1-aza-bicyclo[2.2.2]octyl, 3-aza-bicyclo[3.2.1]octyl, or 2,6-dioxa-tricyclo[3.3.1.0^{3,7}]nonyl), aryl, or heteroaryl.

In several embodiments, R₃ is an optionally substituted phenyl, naphthyl, or a phenyl fused with a C₄₋₈ carbocyclic moiety (e.g., 1,2,3,4-tetrahydronaphthyl, or indanyl).

In several embodiments, R₃ is an aryl including optionally substituted phenyl, naphthalenyl, azulenyl, fluorenyl, or antracenyl.

In several embodiments, R₃ is a phenyl that is substituted at a position meta or ortho relative to the point of attachment between R₂ and the pyrazalone ring with halo, carboxy (e.g., alkoxycarbonyl), amido (e.g., dialkylaminocarbonyl, alkylcarbonylamino, dialkylaminoalkylaminocarbonyl, heterocycloarylaminocarbonyl), amino (e.g., dialkylamino), cyano, cyanoalkyl, alkoxy, sulfamoyl alkylsulfonyl, aminoalkyl, alkyl, hydroxyalkyl, alkoxyalkyl, hydroxyl, carboxyalkyl, dialkylaminoalkyl, sulfonylheterocycloalkyl, alkylsulfonylaminoalkyl, heterocycloalkylcarbonyl, heterocycloalkylcarbonyl, oxoheterocycloalkylcarbonyl, alkylcarbonyl, or haloalkyl.

In several embodiments, R₃ is an optionally substituted phenyl, naphthyl, or a phenyl fused one C₄₋₈ carbocyclic moiety (e.g., 1,2,3,4-tetrahydronaphthyl or indanyl).

In several embodiments, R₃ is a phenyl that is substituted with halo, aminosulfonyl, alkylsulfonylalkyl, hydroxyl, alkylcarbonylamino, amino, alkyl, or alkoxy at a position para relative to the point of attachment between R₃ and the pyrazalone ring.

In several embodiments, R₃ is an unsubstituted methyl, ethyl, propyl, or butyl. In several embodiments, R₃ is a methyl, ethyl, propyl, or butyl each substituted with 1 to 4 halo.

In several embodiments, R₃ is substituted with one or more groups independently selected from aliphatic, cycloaliphatic, (cycloaliphatic)alkyl, (heterocycloaliphatic)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, amino, nitro, carboxy, alkylcarbonyl, amido (e.g., alkylcarbonylamino, cycloalkylcarbonylamino, (cycloalkyl)alkylcarbonylamino, arylcarbonylamino, aralkylcarbonylamino, (heterocycloalkyl)carbonylamino, (heterocycloalkyl)alkylcarbonylamino, heteroarylcarbonylamino, or heteroaralkylcarbonylamino), aminoalkyl, aminosulfonyl, cyano, cyanoalkyl, halo, hydroxy, acyl, mercapto, sulfonyl (such as alkylsulfonyl), sulfinyl, sulfanyl, sulfoxy, urea, thiourea, sulfamoyl, sulfamide, oxo, or carbamoyl.

iv. Substituent R₄

In some embodiments, R₄ is hydrogen, halo, aliphatic, cycloaliphatic, (cycloaliphatic)alkyl, aryl, araliphatic, araliphatic, heterocycloaliphatic, (heterocycloaliphatic)alkyl, heteroaryl, or heteroaraliphatic, each of which is optionally substituted with 1 to 3 of (Y—R₅), where Y and R₅ are defined herein.

In several embodiments, R₄ is hydrogen, halo, aliphatic, cycloaliphatic, (cycloaliphatic)alkyl, aryl, araliphatic, araliphatic, heterocycloaliphatic, (heterocycloaliphatic)alkyl, heteroaryl, or heteroaraliphatic each of which is optionally substituted.

In several embodiments, R₄ is an aliphatic optionally substituted with one to three substituents of (Y—R₅) (e.g., alkyl, alkenyl, alkynyl, (cycloaliphatic)aliphatic, carbonylaliphatic, carboxyaliphatic, alkoxyaliphatic, araliphatic, heteroaraliphatic, sulfonylaliphatic, sulfanylaliphatic, sulfinylaliphatic, carbonylaliphatic, aminoaliphatic, cyanoaliphatic, or heteroaraliphatic); cycloaliphatic (e.g., mono- or bicycloaliphatic); or heterocycloaliphatic (e.g., heterocycloalkyl or heterocycloalkenyl).

In several embodiments, R₄ is a 5-12 membered monocyclic or bicyclic ring system (e.g., 9-11 membered ring system). R₄ can be substituted.

In several embodiments, R₄ is an alkyl including methyl (e.g., trifluoromethyl), ethyl, propyl, isopropyl, butyl, isobutyl, sec-butyl, tert-butyl, n-pentyl, n-heptyl, or 2-ethylhexyl. R₄ is carboxyalkyl, cyanoalkyl, hydroxyalkyl, alkoxyalkyl, carbonylalkyl, carboxyalkyl, hydroxyalkyl, oxoalkyl, aralkyl, alkoxyaralkyl, (alkylsulfonylamino)alkyl, (sulfonylamino)alkyl, carbonylaminoalkyl, haloalkyl, aminocarbonylalkyl, cycloaliphaticalkyl, cyanoalkyl, aminoalkyl, oxoalkyl, or alkoxycarbonylalkyl.

In several embodiments, R₄ is a cycloaliphatic. R₄ is cyclopentyl, cyclohexyl, cyclopentenyl, cyclohexyl, bicyclo[4.3.0]-nonyl, or bicyclo[4.4.0]-decyl. R₄ is substituted with 1-3 substituents including alkyl, alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, amino, nitro, carboxy (e.g., alkoxycarbonyl or alkylcarbonyloxy), alkylcarbonyl, amido (e.g., alkylcarbonylamino, carbonylamino, cycloalkylcarbonylamino, (cycloalkyl)alkylcarbonylamino, arylcarbonylamino, aralkylcarbonylamino, (heterocycloalkyl)carbonylamino, (heterocycloalkyl)alkylcarbonylamino, heteroarylcarbonylamino, or heteroaralkylcarbonylamino), aminoalkyl, aminosulfonyl, cyano, cyanoalkyl, halo, hydroxy, acyl, mercapto, sulfonyl (such as alkylsulfonyl), sulfinyl, sulfanyl, sulfoxy, urea, thiourea, sulfamoyl, sulfamide, oxo, or carbamoyl. In certain embodiments, R₄ is substituted with 1-3 of halo, aliphatic, cycloaliphatic, heterocycloaliphatic, aryl, heteroaryl, hydroxyl, alkoxy, sulfonyl, sulfanyl, or sulfinyl.

In several embodiments, R₄ is aryl. R₄ is an optionally substituted phenyl, naphthyl, or a phenyl fused with one C₄₋₈ carbocyclic moiety (e.g., 1,2,3,4-tetrahydronaphthyl or indanyl). R₄ is further substituted with one or more groups independently selected from alkyl, alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, amino, nitro, carboxy (e.g., alkoxycarbonyl or alkylcarbonyloxy), alkylcarbonyl, amido (e.g., alkylcarbonylamino, carbonylamino, cycloalkylcarbonylamino, (cycloalkyl)alkylcarbonylamino, arylcarbonylamino, aralkylcarbonylamino, (heterocycloalkyl)carbonylamino, (heterocycloalkyl)alkylcarbonylamino, heteroarylcarbonylamino, or heteroaralkylcarbonylamino), aminoalkyl, aminosulfonyl, cyano, cyanoalkyl, halo, hydroxy, acyl, mercapto, sulfonyl (such as alkylsulfonyl), sulfinyl, sulfanyl, sulfoxy, urea, thiourea, sulfamoyl, sulfamide, oxo, or carbamoyl.

In several embodiments, R₄ is an alkyl substituted with 1-3 substituents. R₄ is independently substituted with alkoxycarbonyl, alkylcarbonyl, amino, cyano, hydroxyl, alkoxy, cycloaliphatic, heterocycloaliphatic, aryl, or heteroaryl.

In alternative embodiments, R₃ and R₄ together with the nitrogen atoms to which they are attached form a 5 to 7 membered heterocyclic ring optionally substituted with no more than three substituents, e.g., Y—R₅. In some embodiments, Y is selected from —C(O)—, —C(O)—O—, —O—C(O)—, —S(O)ₚ—O—, —O—S(O)ₚ—, —C(O)—N(R^{b})—, —N(R^{b})—C(O)—, —O—C(O)—N(R^{b})—, —N(R^{b})—C(O)—O—, —O—S(O)ₚ—N(R^{b})—, —N(R^{b})—S(O)ₚ—O—, —N(R^{b})—C(O)—N(R^{c})—, —N(R^{b})—S(O)ₚ—N(R^{c})—, —C(O)—N(R^{b})—S(O)ₚ—, —S(O)ₚ—N(R^{b})—C(O)—, —C(O)—N(R^{b})—S(O)ₚ—N(R^{c})—, —C(O)—O—S(O)ₚ—N(R^{b})—, —N(R^{b})—S(O)ₚ—N(R^{c})—C(O)—, —N(R^{b})—S(O)ₚ—O—C(O)—, —S(O)ₚ—N(R^{b})—, —N(R^{b})—S(O)ₚ—, —N(R^{b})—, —S(O)ₚ—, —O—, —S—, or —(C(R^{b})(R^{c}))_{q}—; where each of R^{b} and R^{c} is independently selected from hydrogen, hydroxy, alkyl, alkoxy, amino, aryl, aralkyl, heterocycloalkyl, heteroaryl, or heteroaralkyl; and p is 1 or 2, and q is 1-4.

In several embodiments, R₄ is a heteroaryl. R₄ is optionally substituted benzo[1,3]dioxolyl, benzo[b]thiophenyl, benzo-oxadiazolyl, benzothiadiazolyl, benzoimidazolyl, benzoxazolyl, benzothiazolyl, 2-oxo-benzoxazolyl, pyridyl, pyrimidinyl, 2,3-dihydro-benzo[1,4]dioxyl, 2,3-dihydro-benzofuryl, 2,3-dihydro-benzo[b]thiophenyl, 3,4-dihydro-benzo[1,4]oxazinyl, 3-oxo-benzo[1,4]oxazinyl, 1,1-dioxo-2,3-dihydro-benzo[b]thiophenyl, [1,2,4]triazolo[1,5-a]pyridyl, [1,2,4]triazolo[4,3-a]pyridyl, quinolinyl, quinoxalyl, quinazolinyl, isoquinolinyl, or cinnolinyl. R₄ is substituted with one or more groups independently selected from alkyl, alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, amino, nitro, carboxy (e.g., alkoxycarbonyl or alkylcarbonyloxy), alkylcarbonyl, amido (e.g., alkylcarbonylamino, carbonylamino, cycloalkylcarbonylamino, (cycloalkyl)alkylcarbonylamino, arylcarbonylamino, aralkylcarbonylamino, (heterocycloalkyl)carbonylamino, (heterocycloalkyl)alkylcarbonylamino, heteroarylcarbonylamino, or heteroaralkylcarbonylamino), aminoalkyl, aminosulfonyl, cyano, cyanoalkyl, halo, hydroxy, acyl, mercapto, sulfonyl (such as alkylsulfonyl), sulfinyl, sulfanyl, sulfoxy, urea, thiourea, sulfamoyl, sulfamide, oxo, or carbamoyl.

v. Substituent R₅

Each R₅ is independently hydrogen, halo, C₁₋₆ aliphatic (e.g., trifluoromethyl, ethyl, ethenyl, ethynyl, n-butyl, or t-butyl), cycloaliphatic (e.g., cycloalkyl, cycloalkenyl, or cycloalkynyl), heterocycloaliphatic (e.g., heterocycloalkyl, heterocycloalkenyl, or heterocycloalkynyl), aryl, or heteroaryl. Each R₅ is independently a 5-12 membered monocyclic, or bicyclic ring. Each R₅ is independently a 9-11 membered monocyclic or bicyclic ring system.

vi. Substituent Y

Each Y is independently a bond, —N(R^{b})—C(O)—, —N(R^{b})—S(O)₂—, —C(O)—, —C(O)—O—, —O—C(O)—, —C(O)—N(R^{b})—, —S(O)ₚ—, —O—, —S(O)₂—N(R^{b})—, —N(R^{b})—, —N(R^{b})—C(O)—O—, —N(R^{b})—C(O)—N(R^{c})—, —C(O)—N(R^{b})—S(O)ₚ—N(R^{c})—, or —C(O)—O—S(O)ₚ—N(R^{b})—, where each of R^{b} and R^{c} is independently selected from hydrogen, hydroxy, aliphatic, alkoxy, amino, carboxy, amido, sulfonylcarbonyl, alkylsulfonyl, aryl, aralkyl, cycloalkyl, heterocycloalkyl, heteroaryl, or heteroaralkyl; and p is 1 or 2, and q is 1-4.

vii. Substituent (Y—R₅)

Without limitation, examples of (Y—R₅) substitutents include (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, amino, nitro, carboxy, alkylcarbonyl, amido (e.g., cycloalkylcarbonylamino, (cycloalkyl)alkylcarbonylamino, arylcarbonylamino, aralkylcarbonylamino, (heterocycloalkyl)carbonylamino, (heterocycloalkyl)alkylcarbonylamino, heteroarylcarbonylamino, (alkylaminoalkylamino)carbonyl, or heteroaralkylcarbonylamino), aminoalkyl, sulfamoyl, cyano, cyanoalkyl, halo, hydroxy, acyl, mercapto, sulfonyl (such as alkylsulfonyl), sulfinyl, sulfanyl, sulfoxy, urea, thiourea, sulfamoyl, sulfamide, oxo, or carbamoyl.

viii. Provisos

In several embodiments, when R₁ is a benzimidazol-6-yl, the nitrogen at the first position of the benzimidazole ring is not substituted with sulfonyl (e.g., alkylsulfonyl or cycloalkylsulfonyl). In still other embodiments, R₁ is not benzimidazolyl substituted with sulfonyl. In other embodiments, R₁ is not benzimidazolyl.

C. Substituent Combinations

In some embodiments, R₁ is a bicylic aryl or bicyclic heteroaryl; R₂ is hydrogen, C₁₋₆ alkyl, aryl, heteroaryl, —C₁₋₄ alkyl-aryl, or —C₁₋₄ alkyl-heteroaryl; R₃ is C₁₋₆ alkyl, C₁₋₂ alkoxy, C₁₋₂ alkyl-carbonyl, C₁₋₂ alkyl-amino, C₁₋₃ alkyl-cycloalkyl, C₁₋₃ alkyl-heterocycloalkyl, C₁₋₃ alkyl-aryl, or C₁₋₃ alkyl-heteroaryl; and R₄ is an alkyl, cycloalkyl, (cycloaliphatic)alkyl, aryl, aralkyl, heterocycloaliphatic, (heterocycloaliphatic)alkyl, heteroaryl, or heteroaraliphatic that is optionally substituted with (Y—R₅), where R₅ is hydrogen, halo, C₁₋₆ alkyl, carbonyl, amino, heterocycloalkyl, aryl, or heteroaryl, and Y is —N(R^{b})—C(O)—, —N(R^{b})—S(O)₂—, —C(O)—, —C(O)—O—, —O—C(O)—, —C(O)—N(R^{b})—, —S(O)ₚ—, —O—, —S(O)₂—N(R^{b})—, —N(R^{b})—, —N(R^{b})—C(O)—O—, —N(R^{b})—C(O)—N(R^{c})—, —C(O)—N(R^{b})—S(O)ₚ—N(R^{c})—, or —C(O)—O—S(O)ₚ—N(R^{b})—.

In other embodiments, R₁ is bicyclic aryl (e.g., naphthalenyl) or bicyclic heteroaryl (e.g., quinoxalyl or benzothiazole); R₂ is aryl (e.g., substituted phenyl), heteroaryl (e.g., furyl, thiophenyl, pyrrolyl, oxazolyl, thiazolyl, imidazolyl, pyrazolyl, isoxazolyl, isothiazolyl, 1,2,3-oxadiazolyl, 1,2,3-triazolyl, 1,3,4-thiadiazolyl, pyridyl, pyridazinyl, pyramidyl, pyrazinyl, 1,3,5-triazyl, thienyl, triazolyl, tetrazolyl, benzyl, benzimidazolyl, or benzthiazolyl); R₃ is C₁₋₆ alkyl, C₁₋₂ alkoxy, C₁₋₂ alkyl-carbonyl, C₁₋₂ alkyl-amino, C₁₋₃ alkyl-cycloalkyl, C₁₋₃ alkyl-heterocycloalkyl, C₁₋₃ alkyl-aryl, or C₁₋₃ alkyl-heteroaryl; and R₄ is an alkyl, cycloalkyl, (cycloaliphatic)alkyl, aryl, aralkyl, heterocycloaliphatic, (heterocycloaliphatic)alkyl, heteroaryl, or heteroaraliphatic that is optionally substituted with 1-3 of (Y—R₅), where R₅ is hydrogen, hydroxyl, alkoxy, halo, C₁₋₆ alkyl, carbonyl, amino, heterocycloalkyl, aryl, or heteroaryl, and Y is —C(O)—, —C(O)—O—, —O—C(O)—, —S(O)ₚ—O—, —O—S(O)ₚ—, —C(O)—N(R^{b})—, —N(R^{b})—C(O)—, —O—C(O)—N(R^{b})—, —N(R^{b})—C(O)—O—, —O—S(O)ₚ—N(R^{b})—, —N(R^{b})—S(O)ₚ—O—, —N(R^{b})—C(O)—N(R^{c})—, —N(R^{b})—S(O)ₚ—N(R^{c})—, —C(O)—N(R^{b})—S(O)ₚ—, —S(O)ₚ—N(R^{b})—C(O)—, —C(O)—N(R^{b})—S(O)ₚ—N(R^{c})—, —C(O)—O—S(O)ₚ—N(R^{b})—, —N(R^{b})—S(O)ₚ—N(R^{c})—C(O)—, —N(R^{b})—S(O)ₚ—O—C(O)—, —S(O)—N(R^{b})—, —N(R^{b})—S(O)ₚ—, —N(R^{b})—, —S(O)ₚ—, —O—, —S—, or —(C(R^{b})(R^{c}))_{q}—; where each of R^{b} and R^{c} is independently selected from hydrogen, hydroxy, alkyl, alkoxy, amino, aryl, aralkyl, heterocycloalkyl, heteroaryl, or heteroaralkyl; and p is 1 or 2, and q is 1-4.

In several embodiments, R₁ is one selected from

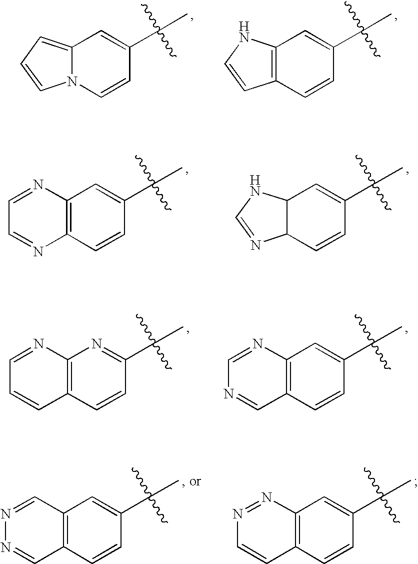

R₂ is aryl (e.g., substituted phenyl), heteroaryl (e.g., furyl, thiophenyl, pyrrolyl, oxazolyl, thiazolyl, imidazolyl, pyrazolyl, isoxazolyl, isothiazolyl, 1,2,3-oxadiazolyl, 1,2,3-triazolyl, 1,3,4-thiadiazolyl, pyridyl, pyridazinyl, pyramidyl, pyrazinyl, 1,3,5-triazyl, thienyl, triazolyl, tetrazolyl, benzyl, benzimidazolyl, or benzthiazolyl); each of which is optionally substituted; R₃ is C₁₋₆ alkyl, C₁₋₂ alkoxy, C₁₋₂ alkyl-carbonyl, C₁₋₂ alkyl-amino, C₁₋₃ alkyl-cycloalkyl, C₁₋₃ alkyl-heterocycloalkyl, C₁₋₃ alkyl-aryl, or C₁₋₃ alkyl-heteroaryl; each of which is optionally substituted; and R₄ is an alkyl, cycloalkyl, (cycloaliphatic)alkyl, aryl, aralkyl, heterocycloaliphatic, (heterocycloaliphatic)alkyl, heteroaryl, or heteroaraliphatic that is optionally substituted with 1-3 of (Y—R₅), where R₅ is hydrogen, halo, C₁₋₆ alkyl, carbonyl, amino, heterocycloalkyl, cycloalkyl (e.g., bicycle[2.2.2]octyl), aryl, or heteroaryl, and Y is —N(R^{b})—C(O)—, —N(R^{b})—S(O)₂—, —C(O)—, —C(O)—O—, —O—C(O)—, —C(O)—N(R^{b})—, —S(O)ₚ—, —O—, —S(O)₂—N(R^{b})—, —N(R^{b})—, —N(R^{b})—C(O)—O—, —N(R^{b})—C(O)—N(R^{c})—, —C(O)—N(R^{b})—S(O)ₚ—N(R^{c})—, or —C(O)—O—S(O)ₚ—N(R^{b})—, where each of R^{b} and R^{c} is independently selected from hydrogen, hydroxy, alkyl, alkoxy, amino, aryl, aralkyl, heterocycloalkyl, heteroaryl, or heteroaralkyl; and p is 1 or 2, and q is 1-4.

Some specific examples of a compound of formula (I) are shown in Examples 1-84 below and include:

TABLE 1
Exemplary Compounds

| Compound | |
|---|---|
| No. | Compound Name |
| 1 | 2-(1,2-Dimethyl-3-oxo-5-quinoxalin-6-yl-2,3-dihydro-1H-pyrazol-4-yl)-benzonitrile |
| 2 | 1,2-Dimethyl-5-quinoxalin-6-yl-4-thiophen-3-yl-1,2-dihydro-pyrazol-3-one |
| 3 | 5-Benzo[1,2,5]thiadiazol-5-yl-1,2-diethyl-4-m-tolyl-1,2-dihydro-pyrazol-3-one |
| 4 | 4-(2-Methyl-5-oxo-3-quinoxalin-6-yl-4-m-tolyl-2,5-dihydro-pyrazol-1-ylmethyl)- |
| | benzoic acid methyl ester |
| 5 | 1-Methyl-5-quinoxalin-6-yl-4-m-tolyl-2-(4-trifluoromethoxy-benzyl)-1,2-dihydro- |
| | pyrazol-3-one |
| 6 | 1-Methyl-5-quinoxalin-6-yl-2-(4-trifluoromethyl-phenyl)-1,2-dihydro-pyrazol-3-one |
| 7 | 1,2-Dimethyl-4-pyridin-2-yl-5-quinoxalin-6-yl-1,2-dihydro-pyrazol-3-one |
| 8 | 2-Pyridin-2-yl-3-quinoxalin-6-yl-6,7-dihydro-5H-pyrazolo[1,2-a]pyrazol-1-one |
| 9 | 2-Pyridin-2-yl-3-quinoxalin-6-yl-5,6,7,8-tetrahydro-pyrazolo[1,2-a]pyridazin-1-one |
| 10 | 1,2-Dimethyl-4-m-tolyl-5-[1,2,4]triazolo[1,5-a]pyridin-6-yl-1,2-dihydro-pyrazol-3- |
| | one |
| 11 | 1,2-Dimethyl-5-quinoxalin-6-yl-4-m-tolyl-1,2-dihydro-pyrazol-3-one |
| 12 | 4-(3-Chloro-phenyl)-1,2-dimethyl-5-quinoxalin-6-yl-1,2-dihydro-pyrazol-3-one |
| 13 | 4-(2-Fluoro-phenyl)-1,2-dimethyl-5-quinoxalin-6-yl-1,2-dihydro-pyrazol-3-one |
| 14 | 1,2-Diethyl-4-pyridin-2-yl-5-quinoxalin-6-yl-1,2-dihydro-pyrazol-3-one |
| 15 | 1,2-Dimethyl-4-pyridin-2-yl-5-quinoxalin-6-yl-1,2-dihydro-pyrazol-3-one |
| 16 | 4-(3-Fluoro-phenyl)-1,2-dimethyl-5-quinoxalin-6-yl-1,2-dihydro-pyrazol-3-one |
| 17 | 1,2-Dimethyl-5-quinoxalin-6-yl-4-(3-trifluoromethyl-phenyl)-1,2-dihydro-pyrazol- |
| | 3-one |
| 18 | 4-(3-Amino-4-fluoro-phenyl)-1,2-dimethyl-5-quinoxalin-6-yl-1,2-dihydro-pyrazol- |
| | 3-one |
| 19 | 1,2-Dimethyl-4-quinolin-6-yl-5-quinoxalin-6-yl-1,2-dihydro-pyrazol-3-one |
| 20 | 4-(3-Dimethylamino-phenyl)-1,2-dimethyl-5-quinoxalin-6-yl-1,2-dihydro-pyrazol- |
| | 3-one |
| 21 | 3-(1,2-Dimethyl-3-oxo-5-quinoxalin-6-yl-2,3-dihydro-1H-pyrazol-4-yl)-benzene |
| | sulfonamide |
| 22 | 4-(4-Amino-phenyl)-1,2-dimethyl-5-quinoxalin-6-yl-1,2-dihydro-pyrazol-3-one |
| 23 | 3-(1,2-Dimethyl-3-oxo-5-quinoxalin-6-yl-2,3-dihydro-1H-pyrazol-4-yl)-benzamide |
| 24 | 1,2-Dimethyl-5-quinoxalin-6-yl-4-thiophen-2-yl-1,2-dihydro-pyrazol-3-one |
| 25 | 4-(3-Acetyl-phenyl)-1,2-dimethyl-5-quinoxalin-6-yl-1,2-dihydro-pyrazol-3-one |
| 26 | 4-(5-Acetyl-thiophen-2-yl)-1,2-dimethyl-5-quinoxalin-6-yl-1,2-dihydro-pyrazol-3- |
| | one |
| 27 | 4-Benzo[b]thiophen-3-yl-1,2-dimethyl-5-quinoxalin-6-yl-1,2-dihydro-pyrazol-3-one |
| 28 | 4-(3-Hydroxymethyl-phenyl)-1,2-dimethyl-5-quinoxalin-6-yl-1,2-dihydro-pyrazol- |
| | 3-one |
| 29 | 3-(1,2-Dimethyl-3-oxo-5-quinoxalin-6-yl-2,3-dihydro-1H-pyrazol-4-yl)-benzonitrile |
| 30 | N-[4-(1,2-Dimethyl-3-oxo-5-quinoxalin-6-yl-2,3-dihydro-1H-pyrazol-4-yl)-phenyl]- |
| | acetamide |
| 31 | 4-(3-Hydroxy-phenyl)-1,2-dimethyl-5-quinoxalin-6-yl-1,2-dihydro-pyrazol-3-one |
| 32 | 4-(4-Hydroxy-phenyl)-1,2-dimethyl-5-quinoxalin-6-yl-1,2-dihydro-pyrazol-3-one |
| 33 | 4-Furan-2-yl-1,2-dimethyl-5-quinoxalin-6-yl-1,2-dihydro-pyrazol-3-one |
| 34 | 4-(3-Bromo-phenyl)-1,2-dimethyl-5-quinoxalin-6-yl-1,2-dihydro-pyrazol-3-one |
| 35 | 4-Benzo[b]thiophen-2-yl-1,2-dimethyl-5-quinoxalin-6-yl-1,2-dihydro-pyrazol-3-one |
| 36 | 4-(1H-Indol-5-yl)-1,2-dimethyl-5-quinoxalin-6-yl-1,2-dihydro-pyrazol-3-one |
| 37 | 4-(1H-Indazol-6-yl)-1,2-dimethyl-5-quinoxalin-6-yl-1,2-dihydro-pyrazol-3-one |
| 38 | 1,2-Dimethyl-4,5-di-quinoxalin-6-yl-1,2-dihydro-pyrazol-3-one |
| 39 | 1-[3-(1,2-Dimethyl-3-oxo-5-quinoxalin-6-yl-2,3-dihydro-1H-pyrazol-4-yl)- |
| | benzoyl]-piperidin-4-one |
| 40 | 1,2-Dimethyl-5-quinoxalin-6-yl-4-[3-(thiomorpholine-4-carbonyl)-phenyl]-1,2- |
| | dihydro-pyrazol-3-one |
| 41 | N-(2-Dimethylamino-ethyl)-3-(1,2-dimethyl-3-oxo-5-quinoxalin-6-yl-2,3-dihydro- |
| | 1H-pyrazol-4-yl)-benzamide |
| 42 | [3-(1,2-Dimethyl-3-oxo-5-quinoxalin-6-yl-2,3-dihydro-1H-pyrazol-4-yl)-phenyl]- |
| | acetonitrile |
| 43 | N-[4-(1,2-Dimethyl-3-oxo-5-quinoxalin-6-yl-2,3-dihydro-1H-pyrazol-4-yl)-benzyl]- |
| | methanesulfonamide |
| 44 | 1,2-Dimethyl-4-[3-(morpholine-4-carbonyl)-phenyl]-5-quinoxalin-6-yl-1,2-dihydro- |
| | pyrazol-3-one |
| 45 | N-[3-(1,2-Dimethyl-3-oxo-5-quinoxalin-6-yl-2,3-dihydro-1H-pyrazol-4-yl)-benzyl]- |
| | methanesulfonamide |
| 46 | 3-(1,2-Dimethyl-3-oxo-5-quinoxalin-6-yl-2,3-dihydro-1H-pyrazol-4-yl)-N-thiazol- |
| | 2-yl-benzamide |
| 47 | 1,2-Dimethyl-4-[2-methyl-5-(morpholine-4-sulfonyl)-phenyl]-5-quinoxalin-6-yl- |
| | 1,2-dihydro-pyrazol-3-one |
| 48 | 4-(3-Dimethylaminomethyl-phenyl)-1,2-dimethyl-5-quinoxalin-6-yl-1,2-dihydro- |
| | pyrazol-3-one |
| 49 | [3-(1,2-Dimethyl-3-oxo-5-quinoxalin-6-yl-2,3-dihydro-1H-pyrazol-4-yl)-phenyl]- |
| | acetic acid |
| 50 | 4-(2-Tert-Butoxymethyl-phenyl)-1,2-dimethyl-5-quinoxalin-6-yl-1,2-dihydro- |
| | pyrazol-3-one |
| 51 | 4-(2-Hydroxy-phenyl)-1,2-dimethyl-5-quinoxalin-6-yl-1,2-dihydro-pyrazol-3-one |
| 52 | 4-(1,2-Dimethyl-3-oxo-5-quinoxalin-6-yl-2,3-dihydro-1H-pyrazol-4-yl)- |
| | benzenesulfonamide |
| 53 | 4-Benzo[1,2,5]oxadiazol-5-yl-1,2-dimethyl-5-quinoxalin-6-yl-1,2-dihydro-pyrazol- |
| | 3-one |
| 54 | 1′-Benzyl-1,2-dimethyl-5-quinoxalin-6-yl-1,2-dihydro-1′H-[4,4′]bipyrazolyl-3-one |
| 55 | 4-(3-Methoxymethyl-phenyl)-1,2-dimethyl-5-quinoxalin-6-yl-1,2-dihydro-pyrazol- |
| | 3-one |
| 56 | 4-(2-Hydroxymethyl-phenyl)-1,2-dimethyl-5-quinoxalin-6-yl-1,2-dihydro-pyrazol- |
| | 3-one |
| 57 | 4-(3-Benzo[1,2,5]thiadiazol-5-yl-5-methoxy-pyrazol-1-yl)-benzoic acid methyl ester |
| 58 | 1,2-Dimethyl-4-phenyl-5-quinoxalin-6-yl-1,2-dihydro-pyrazol-3-one: |
| 59 | 1,2-Dimethyl-4-(6-methyl-pyridin-2-yl)-5-quinoxalin-6-yl-1,2-dihydro-pyrazol-3- |
| | one |
| 60 | 4-(3-Aminophenyl)-1,2-dihydro-1,2-dimethyl-5-(quinoxalin-7-yl)pyrazol-3-one |
| 61 | 1,2-Dihydro-1,2-dimethyl-4-(4-oxo-4H-chromen-6-yl)-5-(quinoxalin-7-yl)pyrazol- |
| | 3-one |
| 62 | 4-(6-Chloropyridin-3-yl)-1,2-dihydro-1,2-dimethyl-5-(quinoxalin-7-yl)pyrazol-3- |
| | one |
| 63 | 4-(3-Amino-4-methylphenyl)-1,2-dihydro-1,2-dimethyl-5-(quinoxalin-7-yl)pyrazol- |
| | 3-one |
| 64 | 4-(3-Amino-4-chlorophenyl)-1,2-dihydro-1,2-dimethyl-5-(quinoxalin-7-yl)pyrazol- |
| | 3-one |
| 65 | 4-(3-(Aminomethyl)phenyl)-1,2-dihydro-1,2-dimethyl-5-(quinoxalin-7-yl)pyrazol- |
| | 3-one |
| 66 | 1,2-Dihydro-1,2-dimethyl-4-(3-(methylsulfonyl)phenyl)-5-(quinoxalin-7-yl)pyrazol- |
| | 3-one |
| 67 | 1,2-Dihydro-1,2-dimethyl-4-(3-(aminosulfonyl)phenyl)-5-(quinoxalin-7-yl)pyrazol- |
| | 3-one |
| 68 | 1,2-Dihydro-4-(3-methoxyphenyl)-1,2-dimethyl-5-(quinoxalin-7-yl)pyrazol-3-one |
| 69 | 2-(2-(2,3-Dihydro-1,2-dimethyl-3-oxo-5-(quinoxalin-7-yl)-1H-pyrazol-4- |
| | yl)phenyl)acetonitrile |
| 70 | N-(3-(2,3-Dihydro-1,2-dimethyl-3-oxo-5-(quinoxalin-7-yl)-1H-pyrazol-4- |
| | yl)phenyl)acetamide |
| 71 | 4-(2-Aminophenyl)-1,2-dihydro-1,2-dimethyl-5-(quinoxalin-7-yl)pyrazol-3-one |
| 72 | 4-(3-Amino-5-nitrophenyl)-1,2-dihydro-1,2-dimethyl-5-(quinoxalin-7-yl)pyrazol-3- |
| | one |
| 73 | 1,2-Dihydro-1,2-dimethyl-4-(quinolin-8-yl)-5-(quinoxalin-7-yl)pyrazol-3-one |
| 74 | Methyl 3-amino-5-(2,3-dihydro-1,2-dimethyl-3-oxo-5-(quinoxalin-7-yl)-1H- |
| | pyrazol-4-yl)benzoate |
| 75 | 1,2-Dihydro-1,2-dimethyl-4-(pyridin-3-yl)-5-(quinoxalin-7-yl)pyrazol-3-one |
| 76 | 4-(3-Chloro-4-fluorophenyl)-1,2-dihydro-1,2-dimethyl-5-(quinoxalin-7-yl)pyrazol- |
| | 3-one |
| 77 | 3-(2,3-Dihydro-1,2-dimethyl-3-oxo-5-(quinoxalin-7-yl)-1H-pyrazol-4-yl)-N,N- |
| | dimethylbenzamide |
| 78 | Methyl 3-(2,3-dihydro-1,2-dimethyl-3-oxo-5-(quinoxalin-7-yl)-1H-pyrazol-4- |
| | yl)benzoate |
| 79 | 4-Furan-3-yl-1,2-dimethyl-5-quinoxalin-6-yl-1,2-dihydro-pyrazol-3-one |
| 80 | 5-Benzo[1,2,5]thiadiazol-5-yl-4-(3-bromo-phenyl)-2-(4-hydroxy-bicyclo[2.2.2]oct- |
| | 1-ylmethyl)-1-methyl-1,2-dihydro-pyrazol-3-one |
| 81 | 4-(3-Ethyl-phenyl)-1,2-dimethyl-5-quinoxalin-6-yl-1,2-dihydro-pyrazol-3-one |
| 82 | 4-(3-Isopropyl-phenyl)-1,2-dimethyl-5-quinoxalin-6-yl-1,2-dihydro-pyrazol-3-one |
| 83 | 1,2-Dimethyl-4-(3-methylsulfanyl-phenyl)-5-quinoxalin-6-yl-1,2-dihydro-pyrazol- |
| | 3-one |
| 84 | 1,2-Dimethyl-5-quinoxalin-6-yl-4-(3-vinyl-phenyl)-1,2-dihydro-pyrazol-3-one |
| 85 | 1,2-Dimethyl-4-(2-methyl-pyridin-4-yl)-5-quinoxalin-6-yl-1,2-dihydro-pyrazol-3- |
| | one |
| 86 | 5-Benzo[1,2,5]thiadiazol-5-yl-1,2-dimethyl-1,2-dihydro-pyrazol-3-one |
| 87 | 5-Benzo[1,2,5]thiadiazol-5-yl-4-bromo-1,2-dimethyl-1,2-dihydro-pyrazol-3-one |
| 88 | 5-Benzo[1,2,5]thiadiazol-5-yl-4-(3-chloro-4-fluoro-phenyl)-1,2-dimethyl-1,2- |
| | dihydro-pyrazol-3-one |
| 89 | 4-(3-Chloro-4-fluoro-phenyl)-1,2-dimethyl-5-[1,2,4]triazolo[1,5-a]pyridin-6-yl-1,2- |
| | dihydro-pyrazol-3-one |
| 90 | 4-m-Tolyl-5-[1,2,4]triazolo[1,5-a]pyridin-6-yl-1,2-dihydro-pyrazol-3-one |
| 91 | 2-Phenyl-4-m-tolyl-5-[1,2,4]triazolo[1,5-a]pyridin-6-yl-1,2-dihydro-pyrazol-3-one |
| 92 | 4-(5-Oxo-4-m-tolyl-3-[1,2,4]triazolo[1,5-a]pyridin-6-yl-2,5-dihydro-pyrazol-1-yl)- |
| | benzenesulfonamide |

An N-oxide derivative or a pharmaceutically acceptable salt of each of the compounds of formula (I) is also within the scope of this invention. For example, a nitrogen ring atom of the imidazole core ring or a nitrogen-containing heterocyclyl substituent can form an oxide in the presence of a suitable oxidizing agent such as m-chloroperbenzoic acid or H₂O₂.

A compound of formula (I) that is acidic in nature (e.g., having a carboxyl or phenolic hydroxyl group) can form a pharmaceutically acceptable salt such as a sodium, potassium, calcium, or gold salt. Also within the scope of the invention are salts formed with pharmaceutically acceptable amines such as ammonia, alkyl amines, hydroxyalkylamines, and N-methylglycamine. A compound of formula (I) can be treated with an acid to form acid addition salts. Examples of such acids include hydrochloric acid, hydrobromic acid, hydroiodic acid, sulfuric acid, methanesulfonic acid, phosphoric acid, p-bromophenyl-sulfonic acid, carbonic acid, succinic acid, citric acid, benzoic acid, oxalic acid, malonic acid, salicylic acid, malic acid, fumaric acid, ascorbic acid, maleic acid, acetic acid, and other mineral and organic acids well known to those skilled in the art. The acid addition salts can be prepared by treating a compound of formula (I) in its free base form with a sufficient amount of an acid (e.g., hydrochloric acid) to produce an acid addition salt (e.g., a hydrochloride salt). The acid addition salt can be converted back to its free base form by treating the salt with a suitable dilute aqueous basic solution (e.g., sodium hydroxide, sodium bicarbonate, potassium carbonate, or ammonia). Compounds of formula (I) can also be, e.g., in a form of achiral compounds, racemic mixtures, optically active compounds, pure diastereomers, or a mixture of diastereomers.

### III. Synthesis of Compounds of Formula (I)

Compounds of formula (I) can be prepared from commercially available starting materials by any known methods. In one method, compounds of formula (I) wherein R₁ is an optionally substituted 8-12 membered saturated, partially unsaturated, or fully unsaturated bicyclic ring system that includes 0-5 heteroatoms selected from O, S, and N, are prepared according to Schemes 1-6 below.

As illustrated in Scheme 1, one method of producing a compound of formula (I) includes reacting a R₁-substituted carboxylic acid (1.1) with methoxymethylamine hydrochloride, in the presence of a coupling agent (e.g., hydroxybenzotriazole, HATU, or PyBOP), base (e.g. diisopropylethylamine), and solvent (eg. dimethylformamide, methylene chloride, or THF) to yield an R₁-substituted alkoxyamide (1.2). See Preparation A below. The R₁-substituted alkoxyamide can be treated with a suitable base (e.g., lithium diisopropylamide, lithium hexamethyldisilazide, diisopropylethylamine, or the like), ethyl acetate, and tetrahydrofuran at a temperature of about −78° C. to yield a R₁-substituted 3-oxo-propionic acid ethyl ester (1.3). See Preparation B below. In turn, the propionic acid ethyl ester (1.3) can react with a R₃, R₄-disubstituted hydrazine hydrochloride refluxed in pyridine to yield a pyrazol-3-one (1.4). See Preparation C below, which illustrates a synthesis using a 1,2-dimethylhydrazine hydrochloride to produce a pyrazal-3-one in which R₃ and R₄ are methyl. The pyrazal-3-one (each of R₃ and R₄ is methyl) can be reacted with a suitable optionally substituted halogenated R₂ (e.g., aryl, heteroaryl, or the like) in the presence of a palladium catalyst (e.g., palladium acetate), tri-(2-furyl)phosphine, and dimethylformamide to produce a compound of formula 1.

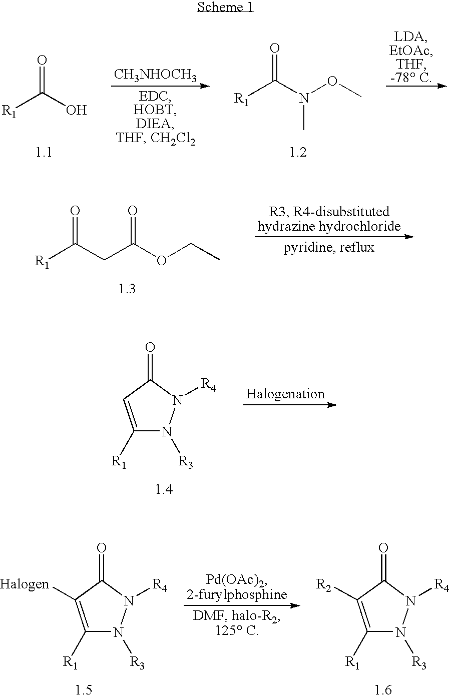

As illustrated in Scheme 2, an alternative method of producing compounds of formula (I) includes using a Weinreb reaction to transform the R₁-substituted carboxylic acid (1.1) to a corresponding Weinreb amide (1.2). See Nahm, S. et al., Tetrahedron Lett., 22, 3815 (1981); Sibi, M. P., Org. Prep. & Proced. Int., 25: 15 (1993); Bailen, M. A. et al., Tetrahedron Lett., 42: 5013 (2001); and Katritzky, A. R. et al., J. Org. Chem., 65: 8069 (2000). The R₁-substituted Weinreb amide (1.2) can be transformed into the 4-halo-1,2-dihydropyrazol-3-one (1.5) using the chemistry shown in Scheme 1 and described above. See Scheme 1 above. The 4-halo-1,2-dihydropyrazol-3-one can undergo a Suzuki reaction at an elevated temperature (e.g., about 110° C.) to produce a compound of formula (I) (1.6) wherein R₂ is an aryl, heteroaryl, cycloaliphatic, or heterocycloaliphatic. See, generally, Suzuki, A., J. Organometallic Chem., 576, 147-168 (1999).

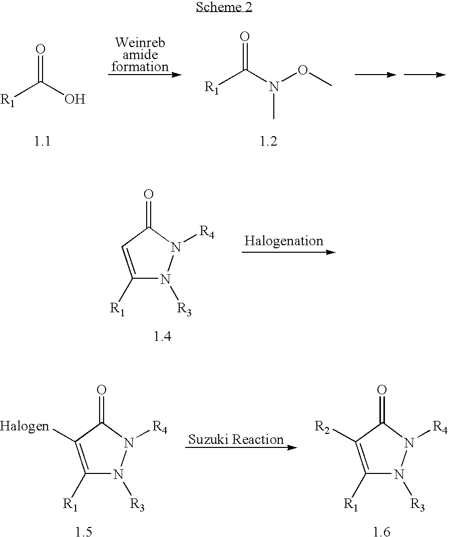

As illustrated in Scheme 3, another alternative method of synthesizing compounds of formula (I) includes treating the alkoxyamide (1.2), formed according to Schemes 1 or 2, with ethylacetate and a suitable base in a solvent at a temperature of about −78° C. to produce the 3-hydroxy-propionic acid ethyl ester (3.1). See Schemes 1-2 above. The ethyl ester (3.1) can be used as an intermediate to produce the compounds of formula (I) by following the pyrazalone formation and substitution described in Schemes 1 or 2.

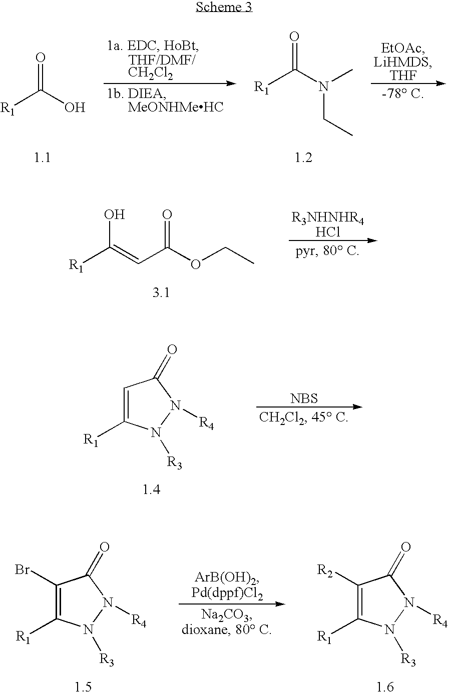

As illustrated in Scheme 4, another method of producing compounds of formula (I) includes reacting a R₁-substituted carboxylic acid (1.1) with a R₃-substituted hydrazine in a solvent to produce the R₁, R₃-disubstituted hydrazine intermediate (4.1). See Scheme 4 and Example 4 below. This disubstituted hydrazine intermediate can then react with a halogenated R₄ (e.g., haloaryl, haloaliphatic, halocycloaliphatic, haloheteroaryl, haloheterocycloaliphatic, or the like) in the presence of a base (e.g., cesium carbonate) and a solvent to produce a R₁, R₃, R₄-trisubstituted hydrazine intermediate (4.2), which can react with acetylchloride in a solvent in the presence of base to produce a hydrazone intermediate (4.3). The hydrazone intermediate (4.3) can then react with a suitable base at −78° C. to form a R₁, R₃, R₄-trisubstituted pyrazal-3-one (1.4), which can be used to produce compounds of formula (I) as described in Schemes 1 and 2.

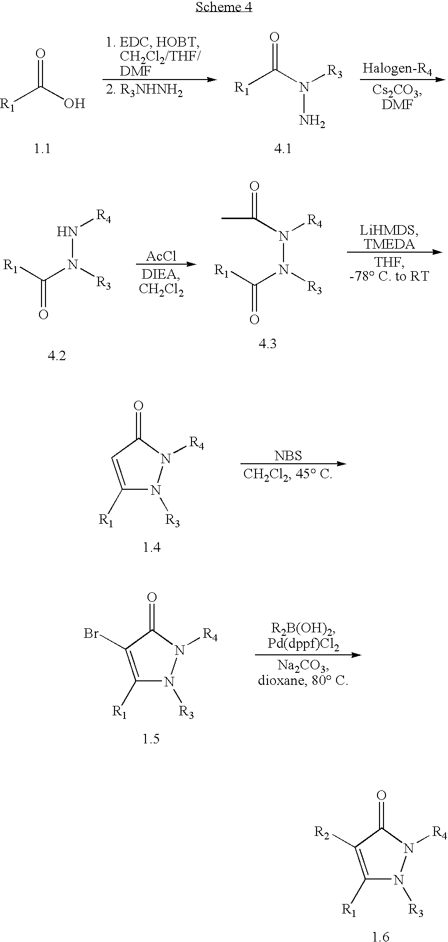

As illustrated in Scheme 5, another method of producing compounds of formula (I) starts with protecting the free amine of a monosubstituted hydrazine (5.1) to produce a protected monosubstituted hydrazine (5.2) (e.g., BOC-protected monosubstituted hydrazine). The protected hydrazine can react with an electrophile (e.g., chloroacetate, bromoacetate, combinations thereof, or the like) to produce a R₄-substituted protected amide (5.3), which is then deprotected to produce the R₄-substituted amide (5.4). The unprotected hydrazide 5.4 is reductively aminated by reaction with an appropriate R₃ aldehyde or ketone followed by reaction with a selective reducing agent (e.g., sodium cyanoborohydride, or the like) and acetic acid to produce an R₃, R₄-disubstituted amide (5.5), which can then react with an R₁-substituted carboxylic acid chloride (5.6) in the presence of a solvent (e.g., dichloromethane) and suitable base (e.g., diisopropylethylamine, or the like) to produce an R₁, R₃, R₄-trisubstituted diamide (4.3). The diamide is treated with a suitable base (e.g., lithium hexamethyldisilazide) in a solvent (e.g., tetrahydrofuran) in the presence of (tetramethylethylenediamine) to produce a R₁, R₃, R₄-trisubstituted pyrazal-3-one (1.4).

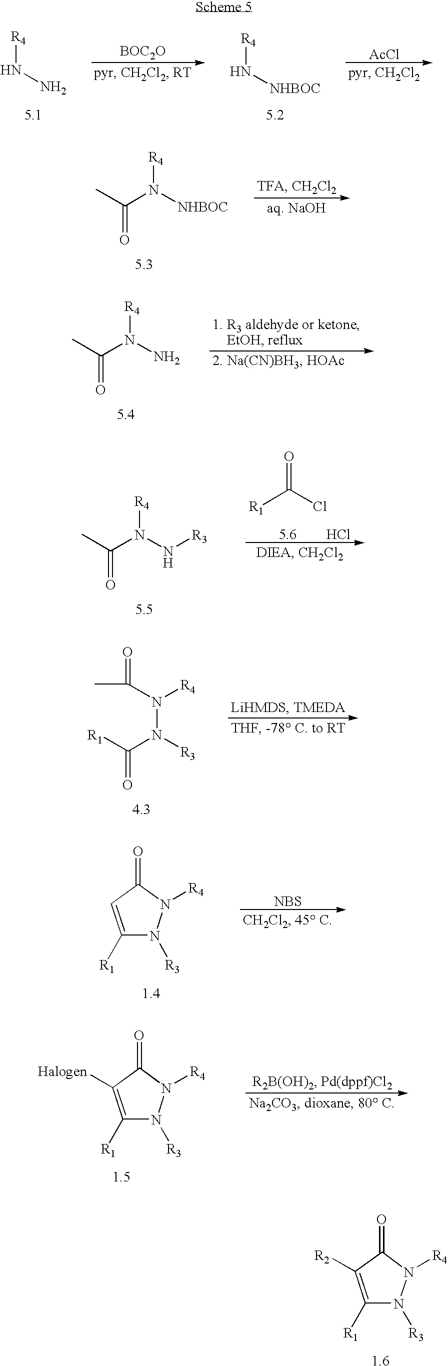

As illustrated in Scheme 6, another method to produce compounds of formula (I) includes reacting a R₂-substituted acid chloride (6.1) with a R₃, R₄-disubstituted hydrazine (e.g., N,N diethylhydrazine hydrochloride) in a solvent (e.g., dichloromethane, or a suitable replacement) in the presence of a base to produce an R₂, R₃, R₄-trisubstituted amide (6.2). The substituted amide can in turn react with a R₁-substituted carboxylic acid chloride (6.3) to produce a R₁, R₂, R₃, R₄-tetrasubstituted diamide (6.4), which can be treated with sodium hydride in the presence of a solvent at a temperature from about 0° C. to about room temperature to produce a compound of formula I.

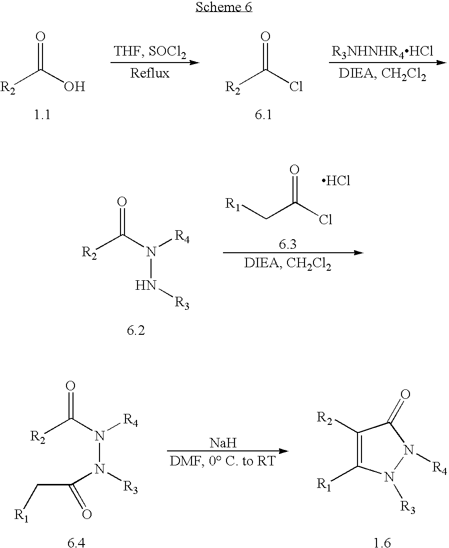

As illustrated in Scheme 7, another method of producing compounds of formula (I) wherein R₃, R₄ and the atoms to which they are attached form an optionally substituted 5-8 membered ring, includes treating a dihaloalkyl (e.g., 1,3-dibromopropane, 1,4-bromobutane, or the like) (7.1) with a protected hydrazine (e.g., BOC-protected hydrazine, or the like) in the presence of tetraethylammonium bromide, aqueous sodium hydroxide, toluene, and dioxane at an elevated temperature (e.g., about 100° C.) to produce a heterocycloalkyl intermediate (e.g. pyrazolidine or the like) (7.2). The heterocycloalkyl can be treated with a R₁-substituted acid chloride (5.6) in a solvent (e.g., dichloromethane, or suitable replacement) in the presence of base to produce an R₁, R₃, R₄-trisubstituted amide (7.3). This trisubstituted amide can then be treated with an R₂-substituted acid chloride (7.4) in the presence of a solvent to produce an R₁, R₂, R₃, R₄-tetrasubstituted diamide (7.4), which can undergo a ring closing reaction to form a compound of formula (I) (7.5)

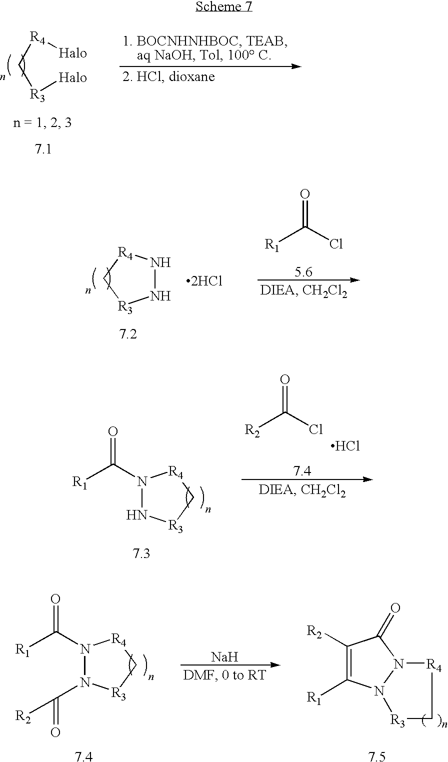

Shown below in Scheme 8 is yet another method for synthesizing compounds of formula (I). This method includes treating an ester (8.1) with an aldehyde (8.2) in a solvent, e.g., tetrahydrofuran (THF), at a low temperature (e.g., at −40° C. and then warmed up to the room temperature) in the presence of lithium diisopropylamide (LDA) to give a β-hydroxy ester (8.3). The resultant ester (8.3) is obtained from the mixture and then dissolved in a solvent (e.g., dichloromethane). To the solution is then added a Dess-Martin reagent for oxidation of the β-hydroxy ester (8.3) into a β-oxo ester (8.4). The β-oxo ester (8.4) is then treated with a hydrazine hydrochloride in pyridine to give a compound of formula (I) (8.5).

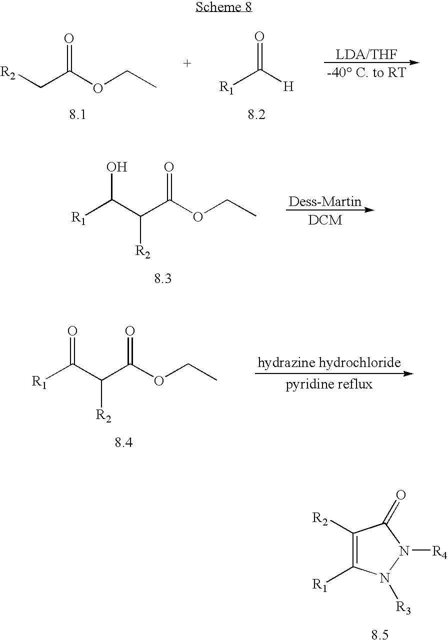

Other desired substitutions can be placed on the pyrazalone ring at positions 1 and 2 by employing a hydrazine including the desired substituents as illustrated in the synthesis schemes above. Moreover, desired substitutions may also be placed on the ring at position 4 by reaction with a halogenated pyrazolone intermediate as illustrated in Schemes 1-5.

### IV. Methods of Use and Compositions

A. Uses of Compounds of formula (I)

As discussed above, hyperactivity of the TGFβ family signaling pathways can result in excess deposition of extracellular matrix and increased inflammatory responses, which can then lead to fibrosis in tissues and organs (e.g., lung, kidney, and liver) and ultimately result in organ failure. See, e.g., Border, W. A. and Ruoslahti E., J. Clin. Invest., 90:1-7 (1992); and Border, W. A. and Noble, N. A. N. Engl. J. Med., 331: 1286-1292 (1994). Studies have been shown that the expression of TGFβ and/or activin mRNA and the level of TGFβ and/or activin are increased in patients suffering from various fibrotic disorders, e.g., fibrotic kidney diseases, alcohol-induced and autoimmune hepatic fibrosis, myelofibrosis, bleomycin-induced pulmonary fibrosis, and idiopathic pulmonary fibrosis.

Compounds of formula (I), which are antagonists of the TGFβ family type I receptors Alk5 and/or Alk 4, and inhibit TGFβ and/or activin signaling pathway, are therefore useful for treating and/or preventing fibrotic disorders or diseases mediated by an increased level of TGFβ and/or activin activity. As used herein, a compound inhibits the TGFβ family signaling pathway when it binds (e.g., with an IC₅₀ value of less than 10 μM; such as, less than 1 μM; and for example, less than 5 nM) to a receptor of the pathway (e.g., Alk5 and/or Alk 4), thereby competing with the endogenous ligand(s) or substrate(s) for binding site(s) on the receptor and reducing the ability of the receptor to transduce an intracellular signal in response to the endogenous ligand or substrate binding. The aforementioned disorders or diseases include any condition (a) marked by the presence of an abnormally high level of TGFβ and/or activin; and/or (b) an excess accumulation of extracellular matrix; and/or (c) an increased number and synthetic activity of myofibroblasts. These disorders or diseases include, but are not limited to, fibrotic conditions such as mesothelioma, acute respiratory distress syndrome (ARDS), atherosclerosis, scleroderma, idiopathic pulmonary fibrosis, keloids, glomerulonephritis, diabetic nephropathy, lupus nephritis, hypertension-induced nephropathy, cholangitis, restinosis, ocular or corneal scarring, hepatic or biliary fibrosis, liver cirrhosis, cirrhosis due to fatty liver disease (alcoholic and nonalcoholic steatosis), renal fibrosis, sarcoidosis, acute lung injury, drug-induced lung injury, spinal cord injury, CNS scarring, systemic lupus erythematosus, Wegener's granulomatosis, pulmonary fibrosis, cardiac fibrosis, post-infarction cardiac fibrosis, post-surgical fibrosis, connective tissue disease, fibrosclerosis, fibrotic cancers, fibroids, fibroma, fibroadenomas, and fibrosarcomas. Other fibrotic conditions for which preventive treatment with compounds of formula (I) can have therapeutic utility include radiation therapy-induced fibrosis, chemotherapy-induced fibrosis, and surgically induced scarring including surgical adhesions, transplant arteriopathy, laminectomy, and coronary restenosis.

Increased TGFβ activity is also found to manifest in patients with progressive cancers. Studies have shown that in late stages of various cancers, both the tumor cells and the stromal cells within the tumors generally overexpress TGFβ. This leads to stimulation of angiogenesis and cell motility, suppression of the immune system, and increased interaction of tumor cells with the extracellular matrix. See, e.g., Hojo, M. et al., Nature, 397: 530-534 (1999). As a result, the tumor cells become more invasive and metastasize to distant organs. See, e.g., Maehara, Y. et al., J. Clin. Oncol., 17: 607-614 (1999) and Picon, A. et al., Cancer Epidemiol. Biomarkers Prev., 7: 497-504 (1998). Thus, compounds of formula (I), which are antagonists of the TGFβ type I receptor and inhibit TGFβ signaling pathways, are also useful for treating and/or preventing various late stage cancers (including carcinomas) which overexpress TGFβ. Such late stage cancers include carcinomas of the lung, breast, liver, biliary tract, gastrointestinal tract, head and neck, pancreas, prostate, cervix, as well as multiple myeloma, melanoma, glioma, and glioblastomas.

Importantly, it should be pointed out that because of the chronic, and in some cases localized, nature of disorders or diseases mediated by overexpression of TGFβ and/or activin (e.g., fibrosis or cancers), small molecule treatments (such as treatment disclosed in the present invention) are favored for long-term treatment.

Not only are compounds of formula (I) useful in treating disorders or diseases mediated by high levels of TGFβ and/or activin activity, these compounds can also be used to prevent the same disorders or diseases. It is known that polymorphisms leading to increased TGFβ and/or activin production have been associated with fibrosis and hypertension. Indeed, high serum TGFβ levels are correlated with the development of fibrosis in patients with breast cancer who have received radiation therapy, chronic graft-versus-host-disease, idiopathic interstitial pneumonitis, veno-occlusive disease in transplant recipients, and peritoneal fibrosis in patients undergoing continuous ambulatory peritoneal dialysis. Thus, the levels of TGFβ and/or activin in serum and of TGFβ and/or activin mRNA in tissue can be measured and used as diagnostic or prognostic markers for disorders or diseases mediated by overexpression of TGFβ and/or activin, and polymorphisms in the gene for TGFβ that determine the production of TGFβ and/or activin can also be used in predicting susceptibility to disorders or diseases. See, e.g., Blobe, G. C. et al., N. Engl. J. Med., 342(18): 1350-1358 (2000); Matsuse, T. et al., Am. J. Respir. Cell Mol. Biol., 13: 17-24 (1995); Inoue, S. et al., Biochem. Biophys. Res. Comm., 205: 441-448 (1994); Matsuse, T. et al, Am. J. Pathol., 148: 707-713 (1996); De Bleser et al., Hepatology, 26: 905-912 (1997); Pawlowski, J. E., et al., J. Clin. Invest., 100: 639-648 (1997); and Sugiyarna, M. et al., Gastroenterology, 114: 550-558 (1998).

In some embodiments, the inhibitors described herein are effective at treating, preventing, or reducing intimal thickening, vascular remodeling, restenosis (e.g., coronary, peripheral, carotid restenosis), vascular diseases, (e.g., intimal thickening, vascular remodeling, organ transplant-related, cardiac, and renal), and hypertension (e.g., primary and secondary, systolic, pulmonary, and hypertension-induced vascular remodeling resulting in target organ damage).

Without wishing to be bound by any particular theory, one possible explanation for the efficacy of the compounds described herein may be their inhibitory effect on the TGFβ and activin pathways.

The pathological activation of the TGFβ and activin pathway plays a critical role in the progression of fibrotic diseases. The critical serine-threonine kinase in the TGFβ type I receptor (TGFβRI) and the activin type I receptor (Alk4) are attractive targets for blockade of the TGFβ pathway for several important reasons. TGFβRI kinase activity is required for TGFβ signaling as is Alk4 for activin signaling. Kinases have proven to be useful targets for development of small molecule drugs. There is a good structural understanding of the TGFβRI kinase domain allowing the use of structure-based drug discovery and design to aid in the development of inhibitors.

TGFβ or activin-mediated pathological changes in vascular flow and tone are often the cause of morbidity and mortality in a number of diseases (Gibbons G. H. and Dzau V. J., N. Eng. J. Med., 330:1431-1438 (1994)). Typically, the initial response of the vasculature to injury is an infiltration of adventitial inflammatory cells and induction of activated myofibroblasts or smooth muscle cells (referred to as myofibroblasts from hereon). TGFβ is initially produced by infiltrating inflammatory cells and activates myofibroblasts or smooth muscle cells. These activated myofibroblasts can also secrete TGFβ as well as respond to it. Within the first few days following injury, myofibroblasts secreting TGFβ migrate from the various layers of the vascular wall towards the lumen where they undergo proliferation and extracellular matrix secretion resulting in intimal thickening. Additionally, TGFβ induces activated myofibroblasts to contract which results in lumenal narrowing. These vascular remodeling processes, intimal thickening and vascular contraction, restrict blood flow to the tissues supported by the effected vasculature and result in tissue damage. Activin is also produced in response to injury and shows very similar actions in inducing activated myofibroblasts or activated smooth muscle cells intimal thickening and vascular remodeling. See, e.g., Pawlowski et al., J. Clin. Invest., 100: 639-648 (1997); Woodruff, T. K., Biochem Pharmacol., 55: 953-963 (1998); Molloy et al., J. Endocrinol., 161(2): 179-85 (1999); and Harada, K. et al., J. Clin. Endocrinol. Metab., 81(6):2125-30 (1996).

In coronary, peripheral or carotid artery disease, balloon angioplasty or stent placement is used to increase lumen size and blood flow. However, the physical damage created by stretching the vessel wall causes injury to the vessel wall tissue. TGFβ elevation following injury induces myofibroblasts in 2-5 days and frequently results in restenosis within 6 months of balloon angioplasty or within a few years of stent placement in human patients. Following balloon angioplasty, both intimal thickening as well vascular remodeling due to myofibroblast contraction, cause narrowing of the lumen and decreased blood flow. Stent placement physically prevents remodeling, but hyperplasia and extracellular matrix deposition by activated myofibroblasts proliferating at the luminal side of the stent results in intimal thickening within the stented vessel resulting in the eventual impairment of blood flow.

The treatment of arterial stenotic disease by surgical grafts, e.g. coronary bypass or other bypass surgery, also can elicit restenosis in the grafted vessel. In particular vein grafts undergo intimal thickening and vascular remodeling through a similar mechanism involving TGFβ-induced intimal thickening and vascular remodeling. In this case, the injury is either due to the overdistention of the thin-walled vein graft placed into an arterial vascular context or due to anastamotic or ischemic injury during the transplantation of the graft.

The loss of patency in arteriovenous or synthetic bridge graft fistulas is another vascular remodeling response involving increased TGFβ production. See e.g., Ikegaya N. et al., J. Am. Soc. Nephrol, 11:928-35 (2000); Heine G. H. et al., Kidney Int., 64:1101-7 (2003). Loss of fistula patency causes complications for renal dialysis or other treatments requiring chronic access to the circulatory system (Ascher E., Ann. Vasc. Surg., 15:89-97 (2001)). Blockade of TGF□ by TGF□RI inhibitors will beneficial for preventing restenosis and extending arteriovenous fistula patency.

Elevated TGFβ is implicated in chronic allograft vasculopathy both in animals and humans. Vascular injury, intimal thickening and vascular remodeling is a characteristic pathology in chronic allograft failure. The fibrotic response in chronic allograft failure initiates in the vasculature of the donor organ. Chronic allograft vasculopathy in allografted hearts often manifests within 5 years of transplantation and is the main cause of death in long term survivors of cardiac transplant. Both early detection of cardiac allograft vasculopathy measured as intimal thickening by intravascular ultrasound as well as the elevation of plasma TGFβ has been suggested as a prognostic marker for late cardiac allograft failure (Mehra M R et al., Am J Transplant., 4:1184 (2004)). Cardiac biopsies of grafted hearts also suggest that graft tissue expression of TGFβ correlates significantly to vasculopathy and the number of rejection episodes (Aziz T et al., J. Thorac. Cardiovasc. Surg., 119: 700 (2000)). Finally, patients with high-producing TGFβ genotypes are more susceptible to earlier onset cardiac-transplant coronary vasculopathy (Densem C G et al., J Heart Lung Transplant., 19:551 (2000); Aziz T et al., J. Thorac. Cardiovasc. Surg., 119: 700 (2000); Holweg C T, Transplantation, 71:1463 (2001)).

Elevation of TGFβ can be induced by ischemic, immune and inflammatory responses to the allograft organ. Animal models of acute and chronic renal allograft rejection identify the elevation of TGFβ as a significant contributor to graft failure and rejection (Nagano, H et al., Transplantation, 63: 1101 (1997); Paul, L. C. et al., Am. J. Kidney Dis., 28: 441 (1996); Shihab F S et al., Kidney Int., 50: 1904 (1996)). Rodent models of chronic allograft nephropathy (CAN) show elevation of TGFβ mRNA and immunostaining. In renal allografts TGFβ immunostaining is strongly positive in interstitial inflammatory and fibrotic cells, but also in blood vessels and glomeruli. In humans, the loss of renal function 1 year post renal allograft correlates with TGFβ staining in the grafted kidney. (Cuhaci, B. et al., Transplantation, 68: 785 (1999)). Graft biopsies show also that renal dysfunction correlates with chronic vascular remodeling, ie vasculopathy, and the degree of TGFβ expression correlates significantly with chronic vasculopathy (Viklicky O. et al., Physiol Res., 52:353 (2003)).

The use of immunosuppressive agents such as cyclosporine A in organ transplantation has not prevented vasculopathy and chronic allograft nephropathy suggesting non-immune mechanisms are involved in allograft failure. In fact, cyclosporina and other immunosuppressants have been shown to induce TGFβ expression and may contribute to vasculopathy (Moien-Afshari F. et al., Pharmacol Ther., 100:141 (2003); Jain S. et al., Transplantation, 69:1759 (2000)).

TGFβ is implicated in chronic allograft rejection in both renal and lung transplants due to the clear TGFβ-related fibrotic pathology of this condition as well as the ability of immune suppressants, esp cyclosporin A, to induce TGFβ (Jain S. et al., Transplantation, 69: 1759 (2000)). TGFβ blockade improved renal function while decreasing collagen deposition, renal TGFβ expression as well as vascular afferent arteriole remodeling in a cyclosporine A-induced renal failure model using an anti-TGFβ monoclonal antibody (Islam M. et al., Kidney Int., 59: 498 (2001); Khanna A. K. et al., Transplantation, 67: 882 (1997)). These data are strongly indicative of a causal role for TGFβ in the development and progression of chonic allograft vasculopathy and chronic allograft failure.

Hypertension is a major cause of morbidity and mortality in the U.S. population affecting approximately 1 in 3 individuals. The effect of hypertension on target organs include increased incidence of cardiac failure, myocardial infarction, stroke, renal failure, aneurysm and microvascular hemorrhage. Hypertension-induced damage to the vasculature results in vascular remodeling and intimal thickening which are a major causative factor in many of these morbidities (Weber W. T., Curr Opin Cardiol., 15:264-72 (2000)). Animal experiments suggest that TGFβ is elevated upon induction of hypertension and anti-TGFβ monoclonal antibody blockade of this pathway decreases blood pressure and renal pathology in hypertensive rats (Xu C. et al., J Vasc Surg., 33:570 (2001); Dahly A. J. et al., Am J Physiol Regul Integr Comp Physiol., 283:R757 (2002)). In humans, plasma TGFβ is elevated in hypertensive individuals compared to normotensive controls and plasma TGFβ is also higher in hypertensive individuals with manifest target organ disease compared to hypertensive individuals without apparent target organ damage (Derhaschnig U. et al., Am J Hypertens., 15:207 (2002); Suthanthiran M., Proc Natl Acad Sci USA, 97:3479 (2000)). There is also evidence suggesting that high TGFβ-producing genotypes of TGFβ are a risk factor for development of hypertension (Lijnen P. J., Am J Hypertens., 16:604 (2003); Suthanthiran M., Proc Natl Acad Sci USA, 97:3479 (2000)). Thus the inhibition of the TGFβ pathway may provide an effective therapeutic approach for hypertension or hypertension-induced organ damage.

The vascular injury response in the pulmonary vasculature results in pulmonary hypertension which can lead to overload of the right heart and cardiac failure (Runo J. R., Loyd J. E., Lancet, 361(9368):1533-44 (2003); Sitbon O. et al., Prog Cardiovasc Dis., 45:115-28 (2002); Jeffery T. K., Morrell, N. W., Cardiovasc Dis., 45:173-202 (2002). Prevention of pulmonary vascular remodeling by TGFβRI inhibitors can be of practical utility in diseases such as primary or secondary pulmonary hypertension (Sitbon O. et al., Prog. Cardiovasc Dis., 45:115-28 (2002); Humbert M. et al., J Am Coll Cardiol., 43:13 S-24S (2004)). Inhibition of the progression of vascular remodeling over time will prevent the progression of pulmonary pathology in these life threatening diseases. Secondary pulmonary hypertension occurs often as a manifestation of scleroderma and is one of the primary causes of morbidity and mortality in scleroderma patients (Denton C. P., Black C. M., Rheum Dis Clin North Am., 29:335-49 (2003)). Pulmonary hypertension is also a sequalae of mixed connective tissue disease, chronic obstructive pulmonary disease (COPD) and lupus erythematosis (Fagan K. A., Badesch D. B., Prog Cardiovasc Dis., 45:225-34 (2002); Presberg K. W., Dincer H. E., Curr Opin Pulm Med., 9:131-8 (2003)).

Many of the diseases described above involving vascular remodeling are particularly severe in diabetic patients (Reginelli J. P., Bhatt D. L., J Invasive Cardiol., 14 Suppl E:2E-10E (2002)). Elevated glucose in diabetes can itself induce TGFβ which leads to the increased vascular remodeling and intimal thickening response to vascular injury (Ziyadeh F. J., Am Soc Nephrol., 15 Suppl 1:S55-7 (2004)). In particular, diabetic patients have significantly higher rates of restenosis, vein graft stenosis, peripheral artery disease, chronic allograft nephropathy and chronic allograft vasculopathy (Reginelli J. P., Bhatt D. L., J Invasive Cardiol. 14 Suppl., E:2E-10E (2002); Eisen H., Ross H., J Heart Lung Transplant., 23:S207-13 (2004); Valentine H., J Heart Lung Transplant., 23:S187-93 (2004)). Thus, blockade of TGFβ is of particular utility in diabetic patients at risk for hypertension-related organ failure, diabetic nephropathy, restenosis or vein graft stenosis in coronary or peripheral arteries, and chronic failure of allograft organ transplants (Endemann D. H. et al., Hypertension, 43(2):399-404 (2004); Ziyadeh F., J Am Soc Nephrol. 15 Suppl., 1:S55-7 (2004); Jerums G. et al., Arch Biochem Biophys., 419:55-62 (2003)).

TGFβRI and Alk4 antagonists are effective at treating, preventing, or reducing intimal thickening, vascular remodeling, restenosis (e.g., coronary, peripheral, carotid restenosis), vascular diseases, (e.g., organ transplant-related, cardiac, and renal), and hypertension (e.g., systolic, pulmonary, and hypertension-induced vascular remodeling resulting in target organ damage). Changes in vascular remodeling and intimal thickening may be qualified by measuring the intimal versus medial vascular thickness.

B. Administration of Compounds of Formula (I)

As defined above, an effective amount is the amount required to confer a therapeutic effect on the treated patient. For a compound of formula (I), an effective amount can range, for example, from about 1 mg/kg to about 150 mg/kg (e.g., from about 1 mg/kg to about 100 mg/kg). Effective doses will also vary, as recognized by those skilled in the art, dependant on route of administration, excipient usage, and the possibility of co-usage with other therapeutic treatments including use of other therapeutic agents and/or radiation therapy.

Compounds of formula (I) can be administered in any manner suitable for the administration of pharmaceutical compounds, including, but not limited to, pills, tablets, capsules, aerosols, suppositories, liquid formulations for ingestion or injection or for use as eye or ear drops, dietary supplements, and topical preparations. The pharmaceutically acceptable compositions include aqueous solutions of the active agent, in an isotonic saline, 5% glucose or other well-known pharmaceutically acceptable excipient. Solubilizing agents such as cyclodextrins, or other solubilizing agents well-known to those familiar with the art, can be utilized as pharmaceutical excipients for delivery of the therapeutic compounds. As to route of administration, the compositions can be administered orally, intranasally, transdermally, intradermally, vaginally, intraaurally, intraocularly, buccally, rectally, transmucosally, or via inhalation, implantation (e.g., surgically), or intravenous administration. The compositions can be administered to an animal (e.g., a mammal such as a human, non-human primate, horse, dog, cow, pig, sheep, goat, cat, mouse, rat, guinea pig, rabbit, hamster, gerbil, or ferret, or a bird, or a reptile, such as a lizard).

In certain embodiments, the compounds of formula I can be administered by any method that permits the delivery of the compound to combat vascular injuries. For instance, the compounds of formula I can be delivered by any method described above. Additionally, the compounds of formula I can be administered by implantation (e.g., surgically) via an implantable device. Examples of implantable devices include, but are not limited to, stents, delivery pumps, vascular filters, and implantable control release compositions. Any implantable device can be used to deliver the compound provided that 1) the device, compound and any pharmaceutical composition including the compound are biocompatible, and 2) that the device can deliver or release an effective amount of the compound to confer a therapeutic effect on the treated patient.

Delivery of therapeutic agents via stents, delivery pumps (e.g., mini-osmotic pumps), and other implantable devices is known in the art. See, e.g., Hofma et al., Current Interventional Cardiology Reports, 3:28-36 (2001), the entire contents of which, including references cited therein, are incorporated herein. Other descriptions of implantable devices, such as stents, can be found in U.S. Pat. Nos. 6,569,195 and 6,322,847; and PCT Publication Nos. WO04/0044405; WO04/0018228; WO03/0229390; WO03/0228346; WO03/0225450; WO03/0216699; and WO03/0204168, each of which is incorporated herein by reference in its entirety.

A delivery device, such as stent, includes a compound of formula I. The compound may be incorporated into or onto the stent using methodologies known in the art. In some embodiments, a stent can include interlocked meshed cables. Each cable can include metal wires for structural support and polyermic wires for delivering the therapeutic agent. The polymeric wire can be dosed by immersing the polymer in a solution of the therapeutic agent. Alternatively, the therapeutic agent can be embedded in the polymeric wire during the formation of the wire from polymeric precursor solutions. In other embodiments, stents or implatable devices can be coated with polymeric coatings that include the therapeutic agent. The polymeric coating can be designed to control the release rate of the therapeutic agent.

Controlled release of therapeutic agents can utilize various technologies. Devices are known having a monolithic layer or coating incorporating a heterogeneous solution and/or dispersion of an active agent in a polymeric substance, where the diffusion of the agent is rate limiting, as the agent diffuses through the polymer to the polymer-fluid interface and is released into the surrounding fluid. In some devices, a soluble substance is also dissolved or dispersed in the polymeric material, such that additional pores or channels are left after the material dissolves. A matrix device is generally diffusion limited as well, but with the channels or other internal geometry of the device also playing a role in releasing the agent to the fluid. The channels can be pre-existing channels or channels left behind by released agent or other soluble substances.

Erodible or degradable devices typically have the active agent physically immobilized in the polymer. The active agent can be dissolved and/or dispersed throughout the polymeric material. The polymeric material is often hydrolytically degraded over time through hydrolysis of labile bonds, allowing the polymer to erode into the fluid, releasing the active agent into the fluid. Hydrophilic polymers have a generally faster rate of erosion relative to hydrophobic polymers. Hydrophobic polymers are believed to have almost purely surface diffusion of active agent, having erosion from the surface inwards. Hydrophilic polymers are believed to allow water to penetrate the surface of the polymer, allowing hydrolysis of labile bonds beneath the surface, which can lead to homogeneous or bulk erosion of polymer.

The implantable device coating can include a blend of polymers each having a different release rate of the therapeutic agent. For instance, the coating can include a polylactic acid/polyethylene oxide (PLA-PEO) copolymer and a polylactic acid/polycaprolactone (PLA-PCL) copolymer. The polylactic acid/polyethylene oxide (PLA-PEO) copolymer can exhibit a higher release rate of therapeutic agent relative to the polylactic acid/polycaprolactone (PLA-PCL) copolymer. The relative amounts and dosage rates of therapeutic agent delivered over time can be controlled by controlling the relative amounts of the faster releasing polymers relative to the slower releasing polymers. For higher initial release rates the proportion of faster releasing polymer can be increased relative to the slower releasing polymer. If most of the dosage is desired to be released over a long time period, most of the polymer can be the slower releasing polymer. The stent can be coated by spraying the stent with a solution or dispersion of polymer, active agent, and solvent. The solvent can be evaporated, leaving a coating of polymer and active agent. The active agent can be dissolved and/or dispersed in the polymer. In some embodiments, the co-polymers can be extruded over the stent body.

In still other embodiments, compounds of formula (I) can be administered in conjunction with one or more other agents that inhibit the TGFβ signaling pathway or treat the corresponding pathological disorders (e.g., fibrosis or progressive cancers) by way of a different mechanism of action. Examples of these agents include angiotensin converting enzyme inhibitors, nonsteroid, steroid anti-inflammatory agents, and chemotherapeutics or radiation, as well as agents that antagonize ligand binding or activation of the TGFβ receptors, e.g., anti-TGFβ, anti-TGFβ receptor antibodies, or antagonists of the TGFβ type II receptors.

The invention will be further described in the following examples, which do not limit the scope of the invention described in the claims.

Optionally, compounds of formula (I) can be administered in conjunction with one or more other agents that inhibit the TGFβ signaling pathway or treat the corresponding pathological disorders (e.g., fibrosis or progressive cancers) by way of a different mechanism of action. Examples of these agents include angiotensin converting enzyme inhibitors, nonsteroid and steroid anti-inflammatory agents, as well as agents that antagonize ligand binding or activation of the TGFβ receptors, e.g., anti-TGFβ, anti-TGFβ receptor antibodies, or antagonists of the TGFβ type II receptors.

The invention will be further described in the following examples, which do not limit the scope of the invention described in the claims.

Synthetic procedures illustrated in Schemes 1-8 above were employed in the preparation of the title compound below.

### V. Examples

Synthesis of exemplary intermediates is described in Examples below.

### Example 1

### 2-(1,2-Dimethyl-3-oxo-5-quinoxalin-6-yl-2,3-dihydro-1H-pyrazol-4-yl)-benzonitrile

### Step 1A: Quinoxaline-6-carboxylic acid methoxy-methyl-amide

A 2000 mL round bottomed flask was charged with 21.0 g (121 mmoles) of quinoxaline-6-carboxylic acid, 32.4 g of 1-(3-dimethylaminopropyl)-3-ethylcarbodiimide hydrochloride (169 mmoles), 22.8 g of 1-hydroxybenzotriazole (169 mmoles), and 24.7 g of N,O-dimethylhydroxylamine hydrochloride (253 mmoles) under a nitrogen atmosphere. To this, 315 mL of tetrahydrofuran and 210 mL of dichloromethane were added. Next, 127 mL of triethylamine (729 mmoles) was charged, and the reaction was allowed to stir for 12 hours. After the reaction was complete, the contents of the flask were concentrated to 1/3 volume under vacuum, and 440 mL of water was added. The mixture was extracted with ethyl acetate (3×200 mL); the subsequent organic fractions were then washed with saturated sodium bicarbonate (1×200 mL) and dried over sodium sulfate. The final product (26.1 g; 99% yield) was obtained after removing the solvent under vacuum. The product was used without further purification.

¹H NMR 300 MHz (CDCl₃) δ 3.35 (s, 3H), 3.49 (s, 3H), 7.97 (d, J=8.7 Hz, 1H), 8.05 (d, J=8.7 Hz, 1H), 8.36 (s, 1H), 8.81 (s, 1H).

M/Z Theoretical: 217.09; M/Z+1 217.77.

### Step 1B: 3-Oxo-3-quinoxalin-6-yl-propionic acid ethyl ester

5.8 mL of 2.0 M Lithium diisopropylamide (11.6 mmoles) in tetrahydrofuran/n-heptane was charged into a dry 250 mL round bottom flask fitted with an addition funnel under an atmosphere of nitrogen at −78° C. The addition funnel was then charged with 0.90 mL of anhydrous ethyl acetate (9.2 mmoles) in 6.5 mL of anhydrous tetrahydrofuran. The ethyl acetate solution was then added dropwise to the solution of lithium diisopropylamide, and was allowed to stir for an additional 20 minutes after all the ethyl acetate was added. The addition funnel was then charged with 1.33 g of quinoxaline-6-carboxylic acid methoxy-methyl-amide (6.12 mmoles) in 7.0 mL of anhydrous tetrahydrofuran. The solution of quinoxaline-6-carboxylic acid methoxy-methyl-amide was added dropwise to the reaction. After allowing the reaction to stir for 2 hours, the contents of the flask were warmed to 0° C., and 10 mL of 10% ammonium chloride in water was added in one portion. The reaction was then warmed to room temperature and concentrated to approximately 15 mL under vacuum. To the crude mixture was added 30 mL of brine followed by 100 mL of dichloromethane, and was allowed to age for 8 to 12 hours in which the product will precipitate. The final product was collected via filtration, washed with water, and dried under vacuum (788 mg, 53% yield). The material was used without further purification.

¹H NMR 300 MHz (DMSO-D6): δ 1.18 (t, J=7.1 Hz, 3H), 4.14 (q, J=7.2 Hz, 2H), 4.43 (s, 2H), 8.20 (d, J=8.7 Hz, 1H), 8.30 (d, J=8.7 Hz, 1H), 8.75 (s, 1H), 9.08 (s, 2H).

M/Z Theoretical: 244.08; M/Z+1 244.66.

### Step 1C: 1,2-Dimethyl-5-quinoxalin-6-yl-1,2-dihydro-pyrazol-3-one

23.4 g of 3-oxo-3-quinoxalin-6-yl-propionic acid ethyl ester (95.6 mmoles) and 19.1 g of dimethylhydrazine dihydrochloride (143 mmoles) were dissolved in 1000 mL of anhydrous pyridine in a 2000 mL round bottom flask equipped with a condenser under an atmosphere of nitrogen. The reaction was heated, and allowed to reflux for 12 hours. The reaction was then cooled to ambient temperature, and the solvent was removed under vacuum. The crude product was dissolved in a 1000 mL of dichloromethane, and washed with saturated sodium bicarbonate (1×250 mL), and dried over sodium sulfate. The final material was obtained via flash chromatography with an Isco system/normal phase column, using a gradient of 100% CH₂Cl₂ to 89% CH₂Cl₂: 11% MeOH (6.1 g; 26% yield).

¹H NMR 300 MHz (DMSO-D6) δ 3.27 (s, 3H), 3.35 (s, 3H), 5.86 (s, 1H), 7.89 (d, J=6.3 Hz, 1H), 8.21-8.24 (m, 2H), 9.04 (s, 2H).

M/Z Theoretical: 240.10; M/Z+240.72.

### Step 1D: 2-(1,2-Dimethyl-3-oxo-5-quinoxalin-6-yl-2,3-dihydro-1H-pyrazol-4-yl)-benzonitrile

47.9 mg of cesium acetate (0.250 mmoles) was charged into an 8 mL vial fitted with an open-top closure and a septa. The base was then heated to 125° C. under vacuum for 2 hours to dry material. Once all the moisture has been removed, the vial is cooled to ambient temperature under an atmosphere of nitrogen. The vial was then charged with 0.3 mg of palladium acetate (1.3×10⁻³ mmoles) and 1.2 mg of tri-(2-furyl)phosphine (5.2×10⁻³ mmoles), and back flushed with nitrogen 3 times. Next, 0.1 mL of anhydrous dimenthylformamide was added, and the solution was allowed to stir until a pale yellow suspension is visible. 30 mg of 1,2-Dimethyl-5-quinoxalin-6-yl-1,2-dihydro-pyrazol-3-one (0.125 mmoles), 27.3 mg of 2-bromobenzonitrile (0.150 mmoles), and 0.26 mL of anhydrous dimethylformamide was charged under an atmosphere of nitrogen in a separate 8 mL vial fitted with a open-top closure and a septa. The substrate solution was then added to the catalyst solution, the vial was sealed, and then the contents heated to 125° C. for 24 hours. After the reaction time was complete, the solvent was removed, and the residue was redissolved in minimal amount of dichloromethane. The filtered dichloromethane solution was then subject to flash chromatography on an Isco flash chromatography system/normal phase column with a gradient of 100% CH₂Cl₂ to 90% CH₂Cl₂: 10% CH₃OH. The final product was obtained in an 87.5% yield, 37.3 mg.

¹H NMR 300 MHz (CDCl₃) δ 3.30 (s, 3H), 3.56 (s, 3H), 7.29 (t, J=5.4 Hz, 1H), 7.48-7.54 (m, 3H), 7.63 (d, J=6.6 Hz, 1H), 7.98 (s, 1H), 8.12 (d, J=6.9 Hz, 1H), 8.86 (d, J=8.7 Hz, 2H).

M/Z Theoretical: 341.13; M/Z+1 341.79.

### Example 2

### 1,2-Dimethyl-5-quinoxalin-6-yl-4-thiophen-3-yl-1,2-dihydro-pyrazol-3-one

4-bromo-1,2-dimethyl-5-quinoxalin-6-yl-1,2-dihydro-pyrazol-3-one (0.235 mmol, Wuxi Pharmatech), 3-thienylboronic acid (45 mg, 0.350 mmol), and [1,1′-bis(diphenylphosphino)ferrocene]dichloropalladium(II), complex with dichloromethane (1:1) (19 mg, 0.024 mmol; Strem) in 1,4-dioxane (2.0 mL, Acros) was dissolved into a vial. To this was added 2.0 M of Sodium carbonate in water (0.50 mL). The reaction was vortexed, sonicated, flushed with argon and sealed. The reaction was shaken for 17 hours at 110° C. under an atmosphere of argon. The reaction was quenched with saturated sodium bicarbonate solution, and extracted with methylene chloride. The organic phases were concentrated, then taken up in DMSO (2.0 mL) and purified by preparative HPLC chromatography. The appropriate fractions were combined and lyophilized to yield 47.8 mg (47%) of 1,2-dimethyl-5-quinoxalin-6-yl-4-thiophen-3-yl-1,2-dihydro-pyrazol-3-one as its trifluoroacetic acid salt.

1H NMR: 9.053 (1H, d, J=1.8 Hz), 9.034 (1H, d, J=1.8 Hz), 8.254 (1H, d, J=8.6 Hz), 8.166 (1H, d, J=2.0 Hz), 7.822 (1H, d of d, J=8.7 Hz, 1.8 Hz), 7.560 (1H, d of d, J=3.0 Hz, 1.3 Hz), 7.298 (1H, d of d, J=5.0 Hz, 3.0 Hz), 6.706 (1H, d of d, J=5.0 Hz, 1.2 Hz), 3.443 (3H, s), 3.184 (3H, s).

MS: m/z=322.77 (M+H).

### Example 3

### 5-Benzo[1,2,5]thiadiazol-5-yl-1,2-diethyl-4-m-tolyl-1,2-dihydro-pyrazol-3-one

### Step 3A: Benzo[1,2,5]thiadiazole-5-carboxylic acid methoxy-methyl-amide

A solution of 2,1,3-benzothiadiazole-5-carboxylic acid (1.0898 g, 6.048 mmole) and N-(3-dimethylaminopropyl)-N′-ethylcarbodiimide hydrochloride (1.3956 g, 7.2703 mmole) in 1:1 methylene chloride/DMF (60 mL) was stirred at room temperature under a nitrogen atmosphere for 15 minutes. N,O-dimethylhydroxylamine hydrochloride (0.7034 g, 7.211 mmole) and DIEA (2.6 mL, 15 mmole) were then added to the brown solution. After 18 hours N,O-dimethylhydroxylamine hydrochloride (0.7109 g, 7.289 mmole) and DIEA (1.4 mL, 8.0 mmole) were added. After an additional 24 hours the reaction was concentrated in vacuo and purified via flash column chromatography (methanol/methylene chloride) to give 0.4341 of a brown oil identified as benzo[1,2,5]thiadiazole-5-carboxylic acid methoxy-methyl-amide.

MS (ESP+) 223.98 (M+1)

### Step 3B: 3-Benzo[1,2,5]thiadiazol-5-yl-3-hydroxy-acrylic acid ethyl ester

A solution of anhydrous ethyl acetate (0.280 mL, 2.87 mmole) in anhydrous THF (2.3 mL) was cannulated into a solution of 1.8 M lithium diisopropylamide/THF (1.60 mL, 2.90 mmole) in anhydrous THF (1.0 mL) at −78° C. under a nitrogen atmosphere. After 50 minutes a solution of benzo[1,2,5]thiadiazole-5-carboxylic acid methoxy-methyl-amide (0.4341 g, 1.944 mmole) in anhydrous THF (3 mL) was cannulated into the enolate solution. The reaction was allowed to slowly warm. After 2 hours the reaction was at 13° C. It was diluted with methylene chloride (90 mL), washed with brine (30 mL), dried (Na₂SO₄), concentrated in vacuo and purified via flash column chromatography (ethyl acetate/hexanes) to give 0.2672 g of a yellow solid identified as 3-benzo[1,2,5]thiadiazol-5-yl-3-hydroxy-acrylic acid ethyl ester.

MS (ESP+) 250.95 (M+1)

### Step 3C: 5-Benzo[1,2,5]thiadiazol-5-yl-1,2-diethyl-1,2-dihydro-pyrazol-3-one

A slurry of 3-benzo[1,2,5]thiadiazol-5-yl-3-hydroxy-acrylic acid ethyl ester (0.2672 g, 1.068 mmole) and 1,2-diethylhydrazine dihydrochloride (0.2701 g, 1.677 mmole) in anhydrous pyridine (7.0 mL) was warmed to 90° C. under a nitrogen atmosphere. After 23 hours the reaction was allowed to cool to room temperature, concentrated in vacuo and purified via flash column chromatography (THF/methylene chloride+1% ammonium hydroxide) to give 0.1457 g of a yellow oil identified as 5-benzo[1,2,5]thiadiazol-5-yl-1,2-diethyl-1,2-dihydro-pyrazol-3-one.

MS (ESP+) 275.05 (M+1)

### Step 3D: 5-Benzo[1,2,5]thiadiazol-5-yl-4-bromo-1,2-diethyl-1,2-dihydro-pyrazol-3-one

NBS (0.1156 g, 0.6500 mmole) was added to a solution of 5-benzo[1,2,5]thiadiazol-5-yl-1,2-diethyl-1,2-dihydro-pyrazol-3-one (0.1457 g, 0.5312 mmole) in methylene chloride (3.5 mL) at room temperature. The reaction was warmed to 45° C. for 2 hours, concentrated in vacuo and purified via flash column chromatography (THF/methylene chloride+1% ammonium hydroxide) to give 0.2102 g of a yellow solid identified as 5-benzo[1,2,5]thiadiazol-5-yl-4-bromo-1,2-diethyl-1,2-dihydro-pyrazol-3-one contaminated with succinamide. The solid was dissolved in ethyl acetate (25 mL), washed with saturated sodium bicarbonate (2×5 mL), water (5 mL) and brine (5 mL), dried (Na₂SO₄), concentrated in vacuo and purified via flash column chromatography (THF/methylene chloride+1% ammonium hydroxide) to give 0.1064 g of a yellow solid identified as 5-benzo[1,2,5]thiadiazol-5-yl-4-bromo-1,2-diethyl-1,2-dihydro-pyrazol-3-one.

MS (ESP+) 352.85 (M+1), 353.86 (M+2), 354.84 (M+3), 355.89 (M+4)

### Step 3E: 5-Benzo[1,2,5]thiadiazol-5-yl-1,2-diethyl-4-m-tolyl-1,2-dihydro-pyrazol-3-one

A solution of 5-benzo[1,2,5]thiadiazol-5-yl-4-bromo-1,2-diethyl-1,2-dihydro-pyrazol-3-one (0.04712 g, 0.1334 mmole) in 1,4-dioxane (2 mL) was added to 3-methylphenylboronic acid (0.01897 g, 0.1395 mmole) and bis(triphenylphosphine)palladium(II) chloride (0.00636 g, 0.0906 mmole) in a sealable tube. The tube was purged with argon, sealed and stirred at room temperature for 1 hour. Degassed 2 M sodium carbonate (0.2 mL, 0.4 mmole) was added and the reaction warmed to 100° C. for 24 hours. The reaction was cooled to room temperature, bis(triphenylphosphine)palladium(II) chloride (0.00972 g) was added and the reaction was warmed to 100° C. for an additional 20 hours. It was then cooled to room temperature, filtered through silica gel, concentrated in vacuo and purified via reverse column HPLC (acetonitrile/water with 0.1% TFA) to give 0.01311 g of a yellow solid identified as the trifluoroacetic acid salt of 5-benzo[1,2,5]thiadiazol-5-yl-1,2-diethyl-4-m-tolyl-1,2-dihydro-pyrazol-3-one.

MS (ESP+) 365.02 (M+1).

### Example 4

### 4-(2-Methyl-5-oxo-3-quinoxalin-6-yl-4-m-tolyl-2,5-dihydro-pyrazol-1-ylmethyl)-benzoic acid methyl ester

### Step 4A: Quinoxaline-6-carboxylic acid N-methyl-hydrazide

HOBT (0.5432 g, 4.020 mmole) and EDC.HCl (0.7732 g, 4.033 mmle) were added to a slurry of 6-quinoxaline carboxylic acid (0.5894 g, 3.384 mmole) in 1:1:2 acetonitrile/THF/DMF (12 mL) at room temperature. The solid slowly dissolved. After 3 hours the solution of activated ester was slowly cannulated into a solution of methylhydrazine (0.370 mL, 6.79 mmole) in acetonitrile (6 mL) at 0° C. After 2 hours the solution was concentrated in vacuo and purified via flash column chromatography (methylene chloride/methanol+1% ammonium hydroxide) to give 0.4258 g of a yellow solid identified as quinoxaline-6-carboxylic acid N-methyl-hydrazide.

MS (ESP+) 203.04 (M+1)

### Step 4B: 4-[N′-Methyl-N′-(quinoxaline-6-carbonyl)-hydrazinomethyl]-benzoic acid methyl ester

Cesium carbonate (1.284 g, 3.942 mmole) was added to a solution of quinoxaline-6-carboxylic acid N-methyl-hydrazide (0.7874 g, 3.894 mmole) in anhydrous DMF (20 mL) at room temperature under a nitrogen atmosphere to give a brown slurry. After 0.5 hour methyl (4-bromomethylbenzyl)benzoate (0.0.8979 g, 3.920 mmole) was added and the reaction stirred for 4 days. The slurry was then filtered and the solid washed with ethyl acetate. The solute was concentrated in vacuo and purified via flash column chromatography (methylene chloride/THF+1% ammonium hydroxide) to give 0.3660 g of a yellow oil identified as 4-[N′-methyl-N′-(quinoxaline-6-carbonyl)-hydrazinomethyl]-benzoic acid methyl ester.

MS (ESP+) 351.41 (M+1)

### Step 4C: Mixture of 4-[N′-methyl-N′-(quinoxaline-6-carbonyl)-hydrazinomethyl]-benzoic acid methyl ester and 4-[N-acetyl-N′-methyl-N′-(quinoxaline-6-carbonyl)-hydrazinomethyl]-benzoic acid methyl ester

DIEA (0.655 mL, 3.76 mmole) and acetyl chloride (0.133 mL, 1.87 mmole) were added to a solution of 4-[N′-methyl-N′-(quinoxaline-6-carbonyl)-hydrazinomethyl]-benzoic acid methyl ester (0.4382 g, 1.251 mmole) in methylene chloride (13 mL) at 0° C. under a nitrogen atmosphere. The reaction was allowed to warm slowly to room temperature. After day additional DIEA (0.655 mL, 3.760 mmole) and acetyl chloride (0.133 mL, 1.872 mmole) were added. After an additional day the reaction was diluted with methylene chloride (13 mL), washed with 10% sodium bicarbonate (8 mL), water (8 mL) and brine (8 mL), dried (Na₂SO₄) and concentrated in vacuo to give 0.4234 g of an orange solid which was identified as a mixture of 4-[N′-methyl-N′-(quinoxaline-6-carbonyl)-hydrazinomethyl]-benzoic acid methyl ester and 4-[N-acetyl-N′-methyl-N′-(quinoxaline-6-carbonyl)-hydrazinomethyl]-benzoic acid methyl ester. Potassium carbonate powder (0.7028 g, 5.085 mmole) and tetraethylammonium bromide (0.1098 g, 0.5224 mmole) were added to a solution of 4-[N′-methyl-N′-(quinoxaline-6-carbonyl)-hydrazinomethyl]-benzoic acid methyl ester and 4-[N-acetyl-N′-methyl-N′-(quinoxaline-6-carbonyl)-hydrazinomethyl]-benzoic acid methyl ester in anhydrous acetonitrile (15 mL) at room temperature under a nitrogen atmosphere. After 10 minutes acetyl chloride (0.370 mL, 5.208 mmole) was added and the reaction warmed to reflux for 21 hours. The reaction was allowed to cool to room temperature, filtered through celite and concentrated in vacuo to give 0.5256 g of a dark oil which was purified via flash column chromatography (methylene chloride/THF+1% ammonium hydroxide) to give 0.2625 g of a yellow foam identified as a mixture of 4-[N′-methyl-N′-(quinoxaline-6-carbonyl)-hydrazinomethyl]-benzoic acid methyl ester and 4-[N-acetyl-N′-methyl-N′-(quinoxaline-6-carbonyl)-hydrazinomethyl]-benzoic acid methyl ester.

MS (ESP+) 351.42 (M+1 s.m.), 394.44 (M prod.)

### Step 4D: 4-(2-Methyl-5-oxo-3-quinoxalin-6-yl-2,5-dihydro-pyrazol-1-ylmethyl)-benzoic acid methyl ester

A solution of 4-[N′-methyl-N′-(quinoxaline-6-carbonyl)-hydrazinomethyl]-benzoic acid methyl ester and 4-[N-acetyl-N′-methyl-N′-(quinoxaline-6-carbonyl)-hydrazinomethyl]-benzoic acid methyl ester (0.2625 g) and TMEDA (0.20 mL, 1.3 mmole) in anhydrous THF (2.9 mL) was cannulated drop-wise into 1.0 M LiHMDS (1.33 mL, 1.33 mmole) at −78° C. under a nitrogen atmosphere. The reaction was allowed to warm to room temperature slowly. After 2 hours the reaction was warmed to 60° C. The reaction was allowed to cool to room temperature after 5 hours, quenched with 10% ammonium chloride, diluted with ethyl acetate (42 mL), washed with brine (10 mL), dried (Na₂SO₄) and concentrated in vacuo to give 0.1088 g of a yellow oil. The oil was purified via flash column chromatography (methylene chloride/THF+1% ammonium hydroxide) to give 0.04032 g of a cream solid identified as impure 4-(2-methyl-5-oxo-3-quinoxalin-6-yl-2,5-dihydro-pyrazol-1-ylmethyl)-benzoic acid methyl ester.

MS (ESP+) 397.06 (M+Na)

### Step 4E: 4-(4-Bromo-2-methyl-5-oxo-3-quinoxalin-6-yl-2,5-dihydro-pyrazol-1-ylmethyl)-benzoic acid methyl ester

NBS (0.00996 g, 0.0560 mmole) was added to a solution of impure 4-(2-methyl-5-oxo-3-quinoxalin-6-yl-2,5-dihydro-pyrazol-1-ylmethyl)-benzoic acid methyl ester (0.04032 g) in methylene chloride (3 mL) at room temperature. The reaction was then warmed to 45° C. After 4 hours the reaction was allowed to cool to room temperature, concentrated in vacuo and purified via flash column chromatography (methylene chloride/THF+1% ammonium hydroxide) to give 0.01223 g of a yellow solid identified as impure 4-(4-bromo-2-methyl-5-oxo-3-quinoxalin-6-yl-2,5-dihydro-pyrazol-1-ylmethyl)-benzoic acid methyl ester.

MS (ESP+) 452.77 (M+1), 453.37 (M+2), 454.25 (M+3), 455.25 (M+4)

### Step 4F: 4-(2-Methyl-5-oxo-3-quinoxalin-6-yl-4-m-tolyl-2,5-dihydro-pyrazol-1-ylmethyl)-benzoic acid methyl ester

Dichloro[1,1′-bis(diphenylphosphino)ferrocene]palladium (II) dichloromethane adduct (0.0122 g, 0.0270 mmole) and m-tolylboronic acid (0.00585 g, 0.0430 mmole) were placed in a sealable tube and purged with argon gas. Impure 4-(4-bromo-2-methyl-5-oxo-3-quinoxalin-6-yl-2,5-dihydro-pyrazol-1-ylmethyl)-benzoic acid methyl ester (0.01223 g) in 1,4-dioxane (2 mL) and 2 M aqueous sodium carbonate (0.054 mL, 0.11 mmole) were added, the tube sealed and warmed to 80° C. After 2 days the reaction was allowed to cool to room temperature, diluted with methylene chloride (20 mL), washed with water (5 mL) and brine (5 mL), dried (Na₂SO₄), concentrated in vacuo and purified via flash column chromatography (methylene chloride/THF+1% ammonium hydroxide) to give 0.00402 g of a tan solid identified as 4-(2-methyl-5-oxo-3-quinoxalin-6-yl-4-m-tolyl-2,5-dihydro-pyrazol-1-ylmethyl)-benzoic acid methyl ester.

MS (ESP+) 465.08 (M+1).

### Example 5

### 1-Methyl-5-quinoxalin-6-yl-4-m-tolyl-2-(4-trifluoromethoxy-benzyl)-1,2-dihydro-pyrazol-3-one

### Step 5A: Quinoxaline-6-carboxylic acid N-methyl-N′-(4-trifluoromethoxy-benzyl)-hydrazide

Cesium carbonate (0.9142 g, 2.806 mmole) was added to a solution of quinoxaline-6-carboxylic acid N-methyl-hydrazide (0.5006 g, 2.476 mmole) in anhydrous DMF (12.4 mL) at room temperature under a nitrogen atmosphere. After 0.5 hour 4-trifluoromethoxybenzyl bromide (0.480 mL, 3.00 mmole) was added and the reaction stirred overnight. The slurry was then filtered and the solid washed with ethyl acetate. The solute was concentrated in vacuo and purified via flash column chromatography (methylene chloride/THF+1% ammonium hydroxide) to give 0.3335 g of a yellow oil identified as quinoxaline-6-carboxylic acid N-methyl-N′-(4-trifluoromethoxy-benzyl)-hydrazide.

MS (ESP+) 377.03 (M+1)

### Step 5B: Mixture of quinoxaline-6-carboxylic acid N-methyl-N′-(4-trifluoromethoxy-benzyl)-hydrazide and quinoxaline-6-carboxylic acid N′-acetyl-N-methyl-N′-(4-trifluoromethoxy-benzyl)-hydrazide

DIEA (0.540 mL, 3.10 mmole) and acetyl chloride (0.110 mL, 1.55 mmole) were added to a solution of quinoxaline-6-carboxylic acid N-methyl-N′-(4-trifluoromethoxy-benzyl)-hydrazide (0.3876 g, 1.030 mmole) in methylene chloride (10 mL) at 0° C. under a nitrogen atmosphere. The reaction was allowed to warm slowly to room temperature. After 2 days the reaction was diluted with methylene chloride (15 mL), washed with 10% sodium bicarbonate (8 mL), water (8 mL) and brine (8 mL), dried (Na₂SO₄) and concentrated in vacuo to give 0.4135 g of an orange oil which was identified as a mixture of quinoxaline-6-carboxylic acid N-methyl-N′-(4-trifluoromethoxy-benzyl)-hydrazide and quinoxaline-6-carboxylic acid N′-acetyl-N-methyl-N′-(4-trifluoromethoxy-benzyl)-hydrazide.

MS (ESP+) 377.24 (M+1 s.m.), 419.24 (M+1 prod.)

### Step 5C: 1-Methyl-5-quinoxalin-6-yl-2-(4-trifluoromethoxy-benzyl)-1,2-dihydro-pyrazol-3-one

A solution of the quinoxaline-6-carboxylic acid N-methyl-N′-(4-trifluoromethoxy-benzyl)-hydrazide and quinoxaline-6-carboxylic acid N′-acetyl-N-methyl-N′-(4-trifluoromethoxy-benzyl)-hydrazide oxaline-6-carboxylic acid N′-acetyl-N-methyl-N′-(4-trifluoromethoxy-benzyl)-hydrazide mixture (0.4611 g, 1.102 mmole) and TMEDA (0.33 mL, 2.2 mmole) in anhydrous THF (2.5 mL) was cannulated drop-wise into 1.0 M LiHMDS (2.2 mL, 2.2 mmole) at −78° C. under a nitrogen atmosphere. After 1.5 hour the reaction was allowed to warm to room temperature. After 2 hours the reaction was warmed to 60° C. and after an additional 1.5 hours warmed to 70° C. The reaction was allowed to cool to room temperature after 1 hour, quenched with 10% ammonium chloride, diluted with ethyl acetate (47 mL), washed with brine (10 mL), dried (Na₂SO₄) and concentrated in vacuo to give 0.4610 g of a dark brown solid. The solid was purified via flash column chromatography (methylene chloride/THF+1% ammonium hydroxide) to give 0.07378 g of a yellow oil identified as 1-methyl-5-quinoxalin-6-yl-2-(4-trifluoromethoxy-benzyl)-1,2-dihydro-pyrazol-3-one.

MS (ESP+) 401.03 (M+1)

### Step 5D: 4-Bromo-1-methyl-5-quinoxalin-6-yl-2-(4-trifluoromethoxy-benzyl)-1,2-dihydro-pyrazol-3-one

NBS (0.03717 g, 0.2088 mmole) was added to a solution of 1-methyl-5-quinoxalin-6-yl-2-(4-trifluoromethoxy-benzyl)-1,2-dihydro-pyrazol-3-one (0.07141 g, 0.1784 mmole) in methylene chloride (3 mL) at room temperature. The reaction was then warmed to 45° C. After 21 hours the reaction was allowed to cool to room temperature, concentrated in vacuo and purified via flash column chromatography (methylene chloride/THF+1% ammonium hydroxide) to give 0.03344 g of an orange solid identified as 4-bromo-1-methyl-5-quinoxalin-6-yl-2-(4-trifluoromethoxy-benzyl)-1,2-dihydro-pyrazol-3-one.

MS (ESP+) 478.99 (M+1), 480.01 (M+2), 481.00 (M+3), 482.00 (M+4)

### Step 5E: 1-Methyl-5-quinoxalin-6-yl-4-m-tolyl-2-(4-trifluoromethoxy-benzyl)-1,2-dihydro-pyrazol-3-one

Dichloro[1,1′-bis(diphenylphosphino)ferrocene]palladium (II) dichloromethane adduct (0.0065 g, 0.008 mmole) and m-tolylboronic acid (0.0138 g, 0.102 mmole) were placed in a sealable tube and purged with argon gas. 4-Bromo-1-methyl-5-quinoxalin-6-yl-2-(4-trifluoromethoxy-benzyl)-1,2-dihydro-pyrazol-3-one (0.0313 g, 0.0653 mmole) in 1,4-dioxane (2 mL) and 2 M sodium carbonate (0.13 mL, 0.26 mmole) were added, the tube sealed and warmed to 80° C. After 2 days the reaction was allowed to cool to room temperature, diluted with methylene chloride (20 mL), washed with water (5 mL) and brine (5 mL), dried (Na₂SO₄), concentrated in vacuo and purified via flash column chromatography (methylene chloride/THF+1% ammonium hydroxide) to give 0.00512 g of yellow solid identified as 1-methyl-5-quinoxalin-6-yl-4-m-tolyl-2-(4-trifluoromethoxy-benzyl)-1,2-dihydro-pyrazol-3-one.

MS (ESP+) 491.05 (M+1).

### Example 6

### 1-Methyl-5-quinoxalin-6-yl-2-(4-trifluoromethyl-phenyl)-1,2-dihydro-pyrazol-3-one

### Step 6A: N′-(4-trifluoromethyl-phenyl)-hydrazinecarboxylic acid tert-butyl ester/N-(4-trifluoromethyl-phenyl)-hydrazinecarboxylic acid tert-butyl ester

Pyridine (2.0 mL, 25 mmole) and 1 M di-tert-butyldicarbonate/THF (23 mL, 20 mmole) were added to a solution of 4-(trifluoromethyl)phenyl hydrazine (4.0164 g, 22.80 mmole) in methylene chloride (240 mL) at room temperature under a nitrogen atmosphere. After 3 days the reaction was washed with 5% citric acid (80 mL), 10% sodium bicarbonate (80 mL) and brine (80 mL), dried (Na₂SO₄), concentrated in vacuo to give 6.0403 g of an orange solid identified via ¹H NHR as a 10/1 mixture of N′-(4-trifluoromethyl-phenyl)-hydrazinecarboxylic acid tert-butyl ester/N-(4-trifluoromethyl-phenyl)-hydrazinecarboxylic acid tert-butyl ester.

MS (ESP+) 203.04 (M-OtBu)

### Step 6B: N′-acetyl-N′-(4-trifluoromethyl-phenyl)-hydrazinecarboxylic acid tert-butyl ester

Acetyl chloride (2.0 mL, 28 mmole) was added to a solution of the 10/1 mixture of N′-(4-trifluoromethyl-phenyl)-hydrazinecarboxylic acid tert-butyl ester/N-(4-trifluoromethyl-phenyl)-hydrazinecarboxylic acid tert-butyl ester (6.0403 g, 21.68 mmole) in 1:1 pyridine/methylene chloride (16 mL) at 0° C. under a nitrogen atmosphere. A precipitate formed immediately. The reaction was allowed to warm to room temperature overnight. The reaction was cooled to 0° C., acetyl chloride (0.5 mL, 7.0 mmole) was added and the reaction allowed to warm to room temperature again. After 24 hours the methylene chloride was removed in vacuo, water (90 mL) was added and the water decanted to give an orange oil. The oil was dissolved in methylene chloride, dried (Na₂SO₄), concentrated in vacuo and purified via flash column chromatography (methylene chloride/THF) to give 3.3344 g of a pale orange solid identified as N′-acetyl-N′-(4-trifluoromethyl-phenyl)-hydrazinecarboxylic acid tert-butyl ester.

MS (ESP+) 319.02 (M+1)

### Step 6C: N-(4-trifluoromethyl-phenyl)-hydrazide

Trifluoroacetic acid (20 mL) was added to a solution of N′-acetyl-N′-(4-trifluoromethyl-phenyl)-hydrazinecarboxylic acid tert-butyl ester (3.3344 g, 10.48 mmole) in methylene chloride (20 mL) at 0° C. The bath was allowed to warm slowly to room temperature. After 2 hours the reaction was concentrated in vacuo to give an orange solid which was slurried in cold 2.4 N sodium hydroxide for 2 minutes, filtered, washed with cold water and air dried to give 1.50473 g of a tan solid identified as acetic acid N-(4-trifluoromethyl-phenyl)-hydrazide.

MS (ESP+) 219.03 (M+1)

### Step 6D: Quinoxaline-6-carboxylic acid N′-acetyl-N-methyl-N′-(4-trifluoromethyl-phenyl)-hydrazide

DIEA (1.10 mL, 6.32 mmole) and quinoxaline-6-carbonyl chloride hydrochloride (0.3972 g, 1.734 mmole) were added to a solution of acetic acid N-(4-trifluoromethyl-phenyl)-hydrazide (0.3660 g, 1.576 mmole) in methylene chloride (7.8 mL) at 0° C. under a nitrogen atmosphere. The reaction was allowed to warm to room temperature slowly. After 19 hours the reaction was diluted with methylene chloride (50 mL), washed with saturated sodium bicarbonate (15 mL) and brine (15 mL), dried (Na₂SO₄), concentrated in vacuo and purified via flash column chromatography (methylene chloride/THF) to give 0.4370 g of a tan solid identified as quinoxaline-6-carboxylic acid N′-acetyl-N-methyl-N′-(4-trifluoromethyl-phenyl)-hydrazide.

MS (ESP+) 388.99 (M+1)

### Step 6E: 1-Methyl-5-quinoxalin-6-yl-2-(4-trifluoromethyl-phenyl)-1,2-dihydro-pyrazol-3-one

A solution of quinoxaline-6-carboxylic acid N′-acetyl-N-methyl-N′-(4-trifluoromethyl-phenyl)-hydrazide (0.4370 g, 1.125 mmole) and TMEDA (0.34 mL, 2.2 mmole) in anhydrous THF (5.1 mL) was cannulated slowly into 1 M lithium hexamethyldisilazide/THF (2.25 mL, 2.2 mmole) at −78° C. under a nitrogen atmosphere. The reaction was allowed to warm slowly to room temperature. After 3 hours the reaction was quenched with 10% ammonium chloride, concentrated in vacuo, diluted with ethyl acetate/methylene chloride/THF, washed with brine (15 mL), dried (Na₂SO₄) and concentrated in vacuo to give 0.9690 g of a brown solid. The solid was purified via flash column chromatography (methylene chloride/THF+1% ammonium hydroxide) to give 0.1722 g of a yellow solid identified as impure 1-methyl-5-quinoxalin-6-yl-2-(4-trifluoromethyl-phenyl)-1,2-dihydro-pyrazol-3-one.

MS (ESP+) 371.00 (M+1)

### Step 6F: 4-Bromo-1-methyl-5-quinoxalin-6-yl-2-(4-trifluoromethyl-phenyl)-1,2-dihydro-pyrazol-3-one

NBS (0.1032 g, 0.5799 mmole) was added to a solution of impure 1-methyl-5-quinoxalin-6-yl-2-(4-trifluoromethyl-phenyl)-1,2-dihydro-pyrazol-3-one (0.1722 g) in methylene chloride (7.7 mL) at room temperature. The reaction was then warmed to 45° C. After 2 hours the reaction was allowed to cool to room temperature, concentrated in vacuo and purified via flash column chromatography (methylene chloride/THF) to give 0.05022 g of an orange solid identified as 4-bromo-1-methyl-5-quinoxalin-6-yl-2-(4-trifluoromethyl-phenyl)-1,2-dihydro-pyrazol-3-one.

MS (ESP+) 448.86 (M+1), 449.86 (M+2), 450.86 (M+3), 451.94 (M+4)

### Step 6G: 1-Methyl-5-quinoxalin-6-yl-4-m-tolyl-2-(4-trifluoromethyl-phenyl)-1,2-dihydro-pyrazol-3-one

Dichloro[1,1′-bis(diphenylphosphino)ferrocene]palladium (II) dichloromethane adduct (0.005 g, 0.006 mmole) and m-tolylboronic acid (0.0259 g, 0.190 mmole) were placed in a sealable tube and purged with argon gas. 4-Bromo-1-methyl-5-quinoxalin-6-yl-2-(4-trifluoromethyl-phenyl)-1,2-dihydro-pyrazol-3-one (0.05022 g, 0.1118 mmole)) in 1,4-dioxane (3 mL) and 2 M aqueous sodium carbonate (0.22 mL, 0.4 mmole) were added, the tube sealed and warmed to 80° C. After 24 hours the reaction was allowed to cool to room temperature, filtered through a celite plug, concentrated in vacuo and purified via flash column chromatography (methylene chloride/THF) to give 0.03995 g of a yellow solid identified as 1-methyl-5-quinoxalin-6-yl-4-m-tolyl-2-(4-trifluoromethyl-phenyl)-1,2-dihydro-pyrazol-3-one.

MS (ESP+) 461.01 (M+1); ¹H NMR (CDCl₃, 400 MHz).

### Example 7

### 1,2-Dimethyl-4-pyridin-2-yl-5-quinoxalin-6-yl-1,2-dihydro-pyrazol-3-one

### Step 7A: Quinoxaline-6-carbonyl chloride

In a 250 mL round bottom flask, quinoxaline-6-carboxylic acid (5.0 g, 0.029 mol) was added with THF (120 mL). SOCl₂ (10.3 mL, 0.15 mol) was added and the mixture was allowed to stir at reflux for 2 hours. The mixture was concentrated to dryness, then azeotroped twice with toluene (2×50 mL). EtOAc was then added and the mixture was filtered and dried on a fritted glass filter under nitrogen to yield 4.6 g of Quinoxaline-6-carbonyl chloride (84%).

¹H NMR 300 MHz (CDCl₃): δ 7.00 (s, 2H), 8.50 (m, 1H), 9.12 (m, 2H).

### Step 7B: Quinoxaline-6-carboxylic acid N,N′-dimethyl-hydrazide

In a 25 mL round bottom flask, 1,2-dimethylhydrazine (690 mg, 5.2 mmol) was added with CH₂Cl₂ (5 mL). DIEA (2 mL, 10.4 mmol) was then added and the mixture was cooled to −78° C. Quinoxaline-6-carbonyl chloride (500 mg, 2.6 mmol) was then added very slowly dropwise as a suspension in CH₂Cl₂ (5 mL). The mixture was diluted with 10 mL of CH₂Cl₂ (5 mL), extracted with water (20 mL), dried with NaSO₄, and rotovapped to dryness and dried on the vacuum line to yield 260 mg of Quinoxaline-6-carboxylic acid N,N′-dimethyl-hydrazide (47%).

¹H NMR 300 MHz (CDCl₃) δ 2.65 (s, 3H), 3.00 (s, 3H), 7.91 (m, 1H), 8.31 (m, 2H), 8.78 (m, 2H).

(M/Z+1) 217.15.

### Step 7C: Quinoxaline-6-carboxylic acid N,N′-dimethyl-N′-(2-pyridin-2-yl-acetyl)-hydrazide

In a round bottom flask, quinoxaline-6-carboxylic acid N,N′-dimethyl-hydrazide (315 mg, 1.45 mmol) was added with CH₂Cl₂ (5 mL) and stirbar. Pyridin-2-yl-acetyl chloride monohydrochloride salt (692 mg, 3.63 mmol) was then added followed by the addition of DIEA (1.2 mL, 7.25 mmol). The product, quinoxaline-6-carboxylic acid N,N′-dimethyl-N′-(2-pyridin-2-yl-acetyl)-hydrazide, was rotovapped to dryness and continued to next step without further purification.

(M/Z+1) 336.22.

### Step 7D: 1,2-Dimethyl-4-pyridin-2-yl-5-quinoxalin-6-yl-1,2-dihydro-pyrazol-3-one

In a round bottom flask, quinoxaline-6-carboxylic acid N,N′-dimethyl-N′-(2-pyridin-2-yl-acetyl)-hydrazide (500 mg, 1.5 mmol) was added with 5 mL of DMF and was stirred at room temperature. NaH (60% w/w, 180 mg, 3 mmol) was added in one portion and the mixture was stirred for 1 hour at room temperature under nitrogen. LCMS showed no starting material. The reaction was quenched with a small amount of water and purified by flash chromatography (90% CH₂Cl₂: 9% MeOH: 1% NH4OH). NMR showed 1,2-dimethyl-4-pyridin-2-yl-5-quinoxalin-6-yl-1,2-dihydro-pyrazol-3-one (50 mg, 11%).

¹H NMR 300 MHz (CDCl₃) δ 2.50 (m, 3H), 3.30 (d, 3H), 7.03 (m, 1H), 7.79 (m, 2H), 8.14 (m, 4H), 9.01 (m, 2H)

(M/Z+1) 318.16. (M+H) 346.03

### Example 8

### 2-Pyridin-2-yl-3-quinoxalin-6-yl-6,7-dihydro-5H-pyrazolo[1,2-a]pyrazol-1-one

### Step 8A: Pyrazolidine-1,2-dicarboxylic acid di-tert-butyl ester

A solution of di-tert-butyl hydrazinodiformate (9.947 g, 42.82 mmole) in anhydrous DMF (100 mL) was added drop-wise to a slurry of 97% sodium hydride (2.353 g, 98.04 mmole) in anhydrous DMF (50 mL) under a nitrogen atmosphere at room temperature. After 0.5 hour 1,3-dibromopropane (4.60 mL, 43.4 mmole) was added and stirred overnight at room temperature. The reaction was then quenched with water (50 mL) and the DMF was removed in vacuo to give a white slurry. The slurry was diluted with ethyl acetate (200 mL), washed with water (70 mL) and brine (70 mL), dried (Na₂SO₄) and concentrated in vacuo to give 9.701 g of a colorless oil. A two phase solution of the oil (1.1529 g) and tetrabutylammonoium bromide (0.126 g, 0.600 mmole) in toluene (7.6 mL) and saturated sodium hydroxide (3.8 mL) was warmed to 100° C. for 2 hours. The reaction was then cooled to room temperature, diluted with ethyl acetate (75 mL), washed with water (25 mL) and brine (25 mL), dried (Na₂SO₄) and concentrated in vacuo to give 1.2704 g of a yellow oil identified as pyrazolidine-1,2-dicarboxylic acid di-tert-butyl ester.

### Step 8B: Pyrazolidine 2 hydrochloride

Hydrogen chloride gas was bubbled through a solution of pyrazolidine-1,2-dicarboxylic acid di-tert-butyl ester (1.3781 g, 5.060 mmole) in 1,4-dioxane (15 mL) at room temperature until a white precipitate formed. The slurry was then diluted with ether, filtered and dried under a nitrogen atmosphere to give 0.5158 g of a white solid identified as pyrazolidine.2 hydrochloride.

### Step 8C: Quinoxaline-6-carbonyl chloride.hydrochloride

Thionyl chloride (10.5 mL, 144 mmole) was added to a slurry of quinoxaline-6-carboxylic acid (5.0394 g, 28.94 mmole) in anhydrous THF at room temperature under a nitrogen atmosphere. The reaction was warmed to reflux for 24 hours, cooled to room temperature, concentrated in vacuo, azeotroped with toluene and dried in vacuo to give a tan solid identified as quinoxaline-6-carbonyl chloride hydrochloride.

### Step 8D: Pyrazolidin-1-yl-quinoxalin-6-yl-methanone

A slurry of quinoxaline-6-carbonyl chloride.hydrochloride (0.3150 g, 1.641 mmole) in methylene chloride (4 mL) was added to a solution of pyrazolidine.2 hydrogen chloride (0.2368 g, 1.633 mmole) and DIEA (1.14 mL, 6.54 mmole) in methylene chloride (8 mL) at −78° C. under a nitrogen atmosphere. After 3 hours the reaction was quenched with methanol and allowed to warm to room temperature. The reaction was diluted with methylene chloride (10 mL), washed with water (2×8 mL) and brine (8 mL), dried (Na₂SO₄) and concentrated in vacuo to give 0.2110 g of a tan solid identified as pyrazolidin-1-yl-quinoxalin-6-yl-methanone.

MS (ESP+) 229.08 (M+1)

### Step 8E: Pyridin-2-yl-acetyl chloride.hydrochloride

Phosphorus pentachloride (5.0546 g, 24.27 mmole) was added portion-wise to a slurry of pyridin-2-yl-acetic acid.hydrochloride (2.104 g, 12.13 mmole) in acetyl chloride (30 mL) at 0° C. under a nitrogen atmosphere. The slurry was then allowed to warm to room temperature overnight. The slurry was cooled to 0° C., quenched with acetone (3.6 mL, 49 mmole) and filtered. The yellow solid was washed with acetyl chloride until the yellow color faded, then washed with ether and dried under a nitrogen atmosphere to give 1.7081 g of a white solid identified as a 1:1 mixture of pyridin-2-yl-acetic acid.hydrochloride/pyridin-2-yl-acetyl chloride.hydrochloride via ¹H NMR.

### Step 8F: 2-Pyridin-2-yl-1-[2-(quinoxaline-6-carbonyl)-pyrazolidin-1-yl]-ethanone

A 1:1 mixture of pyridin-2-yl-acetic acid.hydrochloride/pyridin-2-yl-acetyl chloride.hydrochloride (0.3392 g) was added portion-wise to a solution of pyrazolidin-1-yl-quinoxalin-6-yl-methanone (0.2014 g, 0.8824 mmole) and DIEA (0.615 mL, 3.53 mmole) in methylene chloride (8.8 mL) at room temperature. After 22 hours another portion of the 1:1 mixture of pyridin-2-yl-acetic acid.hydrochloride/pyridin-2-yl-acetyl chloride hydrochloride was added. After an additional 4 hours the reaction was quenched with methanol, diluted to 30 mL with methylene chloride, washed with water (2×8 mL) and brine (8 mL), dried (Na₂SO₄) and concentrated in vacuo to give 0.2672 g of a brown solid identified as impure 2-pyridin-2-yl-1-[2-(quinoxaline-6-carbonyl)-pyrazolidin-1-yl]-ethanone.

MS (ESP+) 348.00 (M+1), MS (ESP−) 345.96 (M−1).

### Step 8G: TFA salt of 2-pyridin-2-yl-3-quinoxalin-6-yl-6,7-dihydro-5H-pyrazolo[1,2-a]pyrazol-1-one

60% Sodium hydride/oil (0.0479 g, 1.20 mmole) was added portion-wise to a solution of impure 2-pyridin-2-yl-1-[2-(quinoxaline-6-carbonyl)-pyrazolidin-1-yl]-ethanone (0.2672 g) in anhydrous DMF (3.5 mL) at 0° C. under a nitrogen atmosphere. The reaction was allowed to warn to room temperature after 1.5 hours and quenched after a further 3 hours with 0.1 M aqueous hydrogen chloride. The reaction was then diluted with methylene chloride (30 mL), washed with brine (6 mL), dried (Na₂SO₄) and concentrated in vacuo to give 0.1627 g brown solid. The solid was purified via reverse phase HPLC (acetonitrile/water and 0.1% TFA) to give 0.0675 g of a brown solid identified as the TFA salt of 2-pyridin-2-yl-3-quinoxalin-6-yl-6,7-dihydro-5H-pyrazolo[1,2-a]pyrazol-1-one.

MS (ESP+) 330.04 (M+1).

### Example 9

### 2-Pyridin-2-yl-3-quinoxalin-6-yl-5,6,7,8-tetrahydro-pyrazolo[1,2-a]pyridazin-1-one

### Step 9A: Tetrahydro-pyridazine-1,2-dicarboxylic acid di-tert-butyl ester

A two phase reaction mixture of di-tert-butyl-hydrazoformate (10.09 g, 43.45 mmole), TEAB (1.45 g, 6.91 mmole) and 1,4-dibromoethane (7.80 mL, 65.3 mmole) in 2/1 toluene/50% aqueous sodium hydroxide (150 mL) was stirred vigorously and warmed to 100° C. A thick white solid formed. After 6 hours the reaction was allowed to cool to room temperature, diluted with ethyl acetate (500 mL), and the organic phase washed with 10% sodium bicarbonate (200 mL), water (200 mL) and brine (200 mL), dried (Na₂SO₄) and concentrated in vacuo to give 15.976 g of a white solid identified as tetrahydro-pyridazine-1,2-dicarboxylic acid di-tert-butyl ester.

MS (ESP+) 309.08 (M+Na)

### Step 9B: Hexahydro-pyridazine-2 hydrochloride

Hydrogen chloride gas was bubbled through a solution of tetrahydro-pyridazine-1,2-dicarboxylic acid di-tert-butyl ester (15.976 g, 55.244 mmole) in 1,4-dioxane (180 mL) at room temperature until a white precipitate formed. The slurry was then diluted with ether, filtered and dried under a nitrogen atmosphere to give 5.1225 g of a light yellow solid identified as hexahydro-pyridazine.2 hydrochloride.

### Step 9C: Quinoxalin-6-yl-(tetrahydro-pyridazin-1-yl)-methanone

A slurry of quinoxaline-6-carbonyl chloride.hydrochloride (0.3207 g, 1.670 mmole) in methylene chloride (4 mL) was added to a solution of hexahydro-pyridazine.2 hydrogen chloride (0.2570 g, 1.616 mmole) and DIEA (1.13 mL, 6.49 mmole) in methylene chloride (8 mL) at −78° C. under a nitrogen atmosphere. After 2 hours the reaction was quenched with methanol and allowed to warm to room temperature. The reaction was diluted with methylene chloride (8 mL), washed with water (2×8 mL) and brine (8 mL), dried (Na₂SO₄) and concentrated in vacuo to give 0.2856 g of a brown solid identified as quinoxalin-6-yl-(tetrahydro-pyridazin-1-yl)-methanone.

MS (ESP+) 243.08 (M+1)

### Step 9D: 2-Pyridin-2-yl-1-[2-(quinoxaline-6-carbonyl)-tetrahydro-pyridazin-1-yl]-ethanone

A 1:1 mixture of pyridin-2-yl-acetic acid.hydrochloride/pyridin-2-yl-acetyl chloride.hydrochloride (0.5730 g) was added portion-wise to a solution of quinoxalin-6-yl-(tetrahydro-pyridazin-1-yl)-methanone (0.2856 g, 1.179 mmole) and DIEA (1.03 mL, 5.91 mmole) in methylene chloride (12 mL) at room temperature under a nitrogen atmosphere. After 22 hours another portion of the 1:1 mixture of pyridin-2-yl-acetic acid.hydrochloride/pyridin-2-yl-acetyl chloride.hydrochloride (0.2336 g) and DIEA (0.205 mL, 1.18 mmole) were added. After an additional 20 hours the reaction was quenched with water, diluted with methylene chloride (20 mL), washed with water (2×10 mL) and brine (10 mL), dried (Na₂SO₄) and concentrated in vacuo to give 0.3143 g of a brown solid identified as impure 2-pyridin-2-yl-1-[2-(quinoxaline-6-carbonyl)-tetrahydro-pyridazin-1-yl]-ethanone.

MS (ESP+) 362.01 (M+1)

### Step 9E: 2-Pyridin-2-yl-3-quinoxalin-6-yl-5,6,7,8-tetrahydro-pyrazolo[1,2-a]pyridazin-1-one

60% Sodium hydride/oil (0.0519 g, 1.30 mmole) was added portion-wise to a solution of impure 2-pyridin-2-yl-1-[2-(quinoxaline-6-carbonyl)-tetrahydro-pyridazin-1-yl]-ethanone (0.2999 g) in anhydrous DMF (4.2 mL) at 0° C. under a nitrogen atmosphere. The reaction was allowed to warm to room temperature immediately and quenched after 2 hours with 0.1 M aqueous hydrogen chloride. The reaction was then diluted with methylene chloride (45 mL), washed with brine (15 mL), dried (Na₂SO₄) and concentrated in vacuo to give 0.1861 g oil. The oil was purified via flash column chromatography (methylene chloride/methanol and 1% ammonium hydroxide) to give 0.0264 g of a brown solid identified as 2-pyridin-2-yl-3-quinoxalin-6-yl-5,6,7,8-tetrahydro-pyrazolo[1,2-a]pyridazin-1-one.

MS (ESP+) 344.06 (M+1).

### Example 10

### 1,2-Dimethyl-4-m-tolyl-5-[1,2,4]triazolo[1,5-a]pyridin-6-yl-1,2-dihydro-pyrazol-3-one

### Step 10A: 3-Hydroxy-2-m-tolyl-3-[1,2,4]triazolo[1,5-a]pyridin-6-yl-propionic acid ethyl ester

To a solution of 2.0 M lithium diisopropylamide in tetrahydrofuran (2.8 mL; Aldrich) and tetrahydrofuran (THF) (3.0 mL; Acros) at −40° C. was added ethyl meta-tolylacetate (1.00 mL, 5.61 mmol; Aldrich) in THF (6.0 mL). The mixture was stirred for 15 minutes at −40° C. To this was added [1,2,4]triazolo[1,5-a]pyridine-6-carbaldehyde (0.830 g, 5.64 mol) in THF (9.0 mL). The material is very insoluble, so additional tetrahydrofuran (4.5 mL; Acros;) was used to transfer the aldehyde. The mixture was stirred for 30 minutes at −40° C. and then for 3 hours at room temperature. The reaction was cooled in an ice bath, then quenched carefully with 1.00 M hydrogen chloride in water (10. mL; Fisher). The solvents were evaporated, and the residue taken up in DMSO. The DMSO solution was purified using preparative HPLC chromatography to yield 883 mg of the title compound.

m/z=326.15 (M+H).

### Step 10B: 3-Oxo-2-m-tolyl-3-[1,2,4]triazolo[1,5-a]pyridin-6-yl-propionic acid ethyl ester

Into a round-bottom flask was added 3-hydroxy-2-m-tolyl-3-[1,2,4]triazolo[1,5-a]pyridin-6-yl-propionic acid ethyl ester (53 mg, 0.16 mmol) and Dess-Martin periodinane (149 mg, 0.351 mmol; Omega) and methylene chloride (3.00 mL; Acros). The reaction was stirred for 45 minutes at room temperature. The reaction was quenched with saturated sodium bicarbonate (±10% sodium thiosulfate), extracted with methylene chloride, and evaporated to yield 53 mg of crude product.

m/z=324.23 (M+H).

### Step 10C: 1,2-Dimethyl-4-m-tolyl-5-[1,2,4]triazolo[1,5-a]pyridin-6-yl-1,2-dihydro-pyrazol-3-one

Into a vial was dissolved 1,2-dimethylhydrazine, dihydrochloride (34 mg, 0.26 mmol; Fluka) and 3-oxo-2-m-tolyl-3-[1,2,4]triazolo[1,5-a]pyridin-6-yl-propionic acid ethyl ester (0.16 mmol) in pyridine (2.5 mL; Acros). The mixture was purged with argon, sealed and was heated at 110° C. for 20 hours. After cooling, an additional dimethylhydrazine, dihydrochloride (152 mg, 1.14 mmol; Fluka) was added and the mixture again stirred at 110° C. for 20 h and then concentrated. The residue was taken up in DMSO and the solution purified by GILSON preparative HPLC to yield the title compound (10.7 mg).

¹H-NMR (300 MHz, DMSO-d6): 9.101 (1H, m), 8.602 (1H, s), 7.931 (1H, d, J=9.2 Hz), 7.495 (1H, d, J=9.2 Hz), 7.237 (1H, m), 7.051 (1H, dd, J=7.7, 7.7 Hz), 6.938 (1H, d, J=7.7 Hz), 3.411 (3H, s), 3.221 (3H, s), 2.167 (3H, s).

m/z=320.23 (M+H).

Additional compounds of formula I, such as listed in Table 2, can be synthesized using known synthetic methods and the synthetic schemes and examples described herein as guidance.

TABLE 2
Experimental Data for Sample Compounds of Formula (I)

| | | | M/Z |
|---|---|---|---|
| No. | Compound Name | ¹H-NMR | (M + H) |
| 11 | 1,2-Dimethyl-5-quinoxalin-6-yl-4- | ¹H NMR (300 MHz, CDCl₃): d 2.21 (s, 3H), | 331.2 |
| | m-tolyl-1,2-dihydro-pyrazol-3-one | 3.07 (s, 3H), 3.40 (s, 3H), 6.90 (m, 3H), | |
| | | 7.14 (bs, 1H), 7.65 (m, 2H), 8.06 (m, 2H), 8.95 (m, | |
| | | 2H) | |
| 12 | 4-(3-Chloro-phenyl)-1,2-dimethyl-5- | ¹H NMR (300 MHz, CDCl₃): d 3.20 (s, 3H), | 351.2. |
| | quinoxalin-6-yl-1,2-dihydro-pyrazol- | 3.33 (s, 3H), 7.01 (m, 1H), 7.14 (m, 2H), | |
| | 3-one | 7.52 (s, 1H), 7.78 (m, 1H), 8.16 (s, 1H), 8.23 (d, | |
| | | 1H), 9.04 (m, 2H) | |
| 13 | 4-(2-Fluoro-phenyl)-1,2-dimethyl-5- | ¹H NMR 300 MHz (CDCl₃): d 3.22 (s, 3H), | 353.2 |
| | quinoxalin-6-yl-1,2-dihydro-pyrazol- | 3.30 (s, 3H), 7.02 (m, 1H), 7.16 (m, 2H), | |
| | 3-one | 7.40 (m, 1H), 7.70 (m, 1H), 7.98 (s, 1H), 8.15 (d, | |
| | | 1H) | |
| 14 | 1,2-Diethyl-4-pyridin-2-yl-5- | | 346.03 |
| | quinoxalin-6-yl-1,2-dihydro-pyrazol- | | |
| | 3-one | | |
| 15 | 1,2-Dimethyl-4-pyridin-2-yl-5- | ¹H NMR 300 MHz (CDCl₃): d 2.50 (m, 3H), | 318.16 |
| | quinoxalin-6-yl-1,2-dihydro-pyrazol- | 3.30 (d, 3H), 7.03 (m, 1H), 7.79 (m, 2H), | |
| | 3-one | 8.14 (m, 4H), 9.01 (m, 2H) | |
| 16 | 4-(3-Fluoro-phenyl)-1,2-dimethyl-5- | ¹H NMR (400 MHz, DMSO-d6): 9.041 (1H, | 334.8 |
| | quinoxalin-6-yl-1,2-dihydro-pyrazol- | d, J = 1.7 Hz), 9.020 (1H, d, J = 1.8 Hz), | |
| | 3-one | 8.222 (1H, d, J = 8.6 Hz), 8.152 (1H, d, J = 1.9 Hz), | |
| | | 7.765 (1H, d of d, J = 8.6 Hz, 1.9 Hz), 7.218 (1H, | |
| | | d, J = 11.2 Hz), 7.138 (1H, m), 6.940-6.830 (2H, | |
| | | m), 3.471 (3H, s), 3.261 (3H, s). | |
| 17 | 1,2-Dimethyl-5-quinoxalin-6-yl-4- | ¹H NMR (300 MHz, CDCl3): d 3.08 (bs, 3H), | 385.3 |
| | (3-trifluoromethyl-phenyl)-1,2- | 3.25 (bs, 3H), 6.83 (s, 1H), 7.50 (m, 6H), | |
| | dihydro-pyrazol-3-one | 8.18 (m, 1H), 8.85 (m, 1H). | |
| 18 | 4-(3-Amino-4-fluoro-phenyl)-1,2- | | 349.93 |
| | dimethyl-5-quinoxalin-6-yl-1,2- | | |
| | dihydro-pyrazol-3-one | | |
| 19 | 1,2-Dimethyl-4-quinolin-6-yl-5- | | 367.96 |
| | quinoxalin-6-yl-1,2-dihydro-pyrazol- | | |
| | 3-one | | |
| 20 | 4-(3-Dimethylamino-phenyl)-1,2- | | 360.01 |
| | dimethyl-5-quinoxalin-6-yl-1,2- | | |
| | dihydro-pyrazol-3-one | | |
| 21 | 3-(1,2-Dimethyl-3-oxo-5- | | 409.93 |
| | quinoxalin-6-yl-2,3-dihydro-1H- | | |
| | pyrazol-4-yl)-benzene sulfonamide | | |
| 22 | 4-(4-Amino-phenyl)-1,2-dimethyl-5- | | 331.93 |
| | quinoxalin-6-yl-1,2-dihydro-pyrazol- | | |
| | 3-one | | |
| 23 | 3-(1,2-Dimethyl-3-oxo-5- | ¹H NMR (400 MHz, DMSO-d6): 9.026 (1H, | 359.76 |
| | quinoxalin-6-yl-2,3-dihydro-1H- | d, J = 1.9 Hz), 9.006 (1H, m), 8.182 (1H, d, | |
| | pyrazol-4-yl)-benzamide | J = 8.6 Hz), 8.103 (1H, m), 7.900-7.749 (1H, | |
| | | m), 7.725 (1H, d of d, J = 8.8 Hz, 1.9 Hz), | |
| | | 7.588 (1H, d, J = 7.5 Hz), 7.288 (1H, d, J = 7.8 Hz), | |
| | | 3.456 (3H, s), 3.232 (3H, s). | |
| 24 | 1,2-Dimethyl-5-quinoxalin-6-yl-4- | ¹H NMR (400 MHz, DMSO-d6): | 322.18 |
| | thiophen-2-yl-1,2-dihydro-pyrazol- | 9.098-9.022 (2H, m), 8.286 (1H, d, J = 8.7 Hz), 8.241 (1H, | |
| | 3-one | m), 7.880 (1H, d of d, J = 8.5 Hz, 1.9 Hz, | |
| | | 7.168 (1H, d of d, J = 5.1 Hz, 1.3 Hz), 6.978 (1H, d of | |
| | | d, J = 3.6 Hz, 1.1 Hz), 6.832 (1H, d of d, | |
| | | J = 5.1 Hz, 3.8 Hz), 3.445 (3H, m), 3.221 (3H, | |
| | | m). | |
| 25 | 4-(3-Acetyl-phenyl)-1,2-dimethyl-5- | ¹H NMR (400 MHz, DMSO-d6): 9.035 (1H, | 358.79 |
| | quinoxalin-6-yl-1,2-dihydro-pyrazol- | d, J = 1.8 Hz), 9.012 (1H, d, J = 1.7 Hz), | |
| | 3-one | 8.211 (1H, d, J = 8.5 Hz), 8.151 (1H, d, J = 1.6 Hz), | |
| | | 7.877 (1H, m), 7.764 (1H, d of d, J = 8.6 Hz, | |
| | | 1.9 Hz), 7.672 (1H, d, J = 7.8 Hz), 7.475 (1H, d, | |
| | | 8.1 Hz), 7.294 (1H, d of d, J = 7.6 Hz, 7.6 Hz), | |
| | | 3.479 (3H, s), 3.274 (3H, s), 2.303 (3H, s). | |
| 26 | 4-(5-Acetyl-thiophen-2-yl)-1,2- | ¹H NMR (400 MHz, DMSO-d6): 9.101 (1H, | 364.71 |
| | dimethyl-5-quinoxalin-6-yl-1,2- | d, J = 1.8 Hz), 9.074 (1H, d, J = 1.7 Hz), | |
| | dihydro-pyrazol-3-one | 8.336 (1H, d, J = 8.6 Hz), 8.302 (1H, d, J = 1.9 Hz), | |
| | | 7.916 (1H, d of d, J = 8.5 Hz, 1.9 Hz), 7.606 (1H, | |
| | | d, J = 4.3 Hz), 6.911 (1H, d, J = 4.1 Hz), | |
| | | 3.495 (3H, s), 3.336 (3H, s), 2.354 (3H, s). | |
| 27 | 4-Benzo[b]thiophen-3-yl-1,2- | ¹H NMR (400 MHz, DMSO-d6): 8.965 (1H, | 372.77 |
| | dimethyl-5-quinoxalin-6-yl-1,2- | d, J = 1.8 Hz), 8.940 (1H, d, J = 1.8 Hz), | |
| | dihydro-pyrazol-3-one | 8.083 (1H, d, J = 8.7 Hz), 8.026 (1H, d, J = 1.7 Hz), | |
| | | 7.916 (1H, d, J = 7.9 Hz), 7.683 (1H, d of d, | |
| | | J = 8.7 Hz, 1.8 Hz), 7.496 (1H, d, J = 7.8 Hz), | |
| | | 7.426 (1H, s), 7.273 (1H, d of d, J = 7.2 Hz, | |
| | | 7.2 Hz), 7.183 (1H, d of d, 7.5 Hz, 7.5 Hz), | |
| | | 3.502 (3H, s), 3.333 (3H, s). | |
| 28 | 4-(3-Hydroxymethyl-phenyl)-1,2- | ¹H NMR (400 MHz, DMSO-d6): 9.016 (1H, | 346.81 |
| | dimethyl-5-quinoxalin-6-yl-1,2- | m), 8.996 (1H, m), 8.168 (1H, d, J = 8.6 Hz), | |
| | dihydro-pyrazol-3-one | 8.093 (1H, m), 7.715 (1H, m), 7.321 (1H, m), | |
| | | 7.206 (1H, m), 7.060 (1H, m), 6.968 (1H, m), | |
| | | 4.319 (2H, s), 3.452 (3H, m), 3.218 (3H, m). | |
| 29 | 3-(1,2-Dimethyl-3-oxo-5- | ¹H NMR (400 MHz, DMSO-d6): 9.050 (1H, | 341.75 |
| | quinoxalin-6-yl-2,3-dihydro-1H- | m), 9.030 (1H, m), 8.232 (1H, d, J = 8.7 Hz), | |
| | pyrazol-4-yl)-benzonitrile | 8.171 (1H, m), 7.843 (1H, m), 7.766 (1H, d, | |
| | | J = 8.5 Hz), 7.534 (1H, d, J = 7.3 Hz), | |
| | | 7.382-7.281 (2H, m), 3.481 (3H, s), 3.297 (3H, s). | |
| 30 | N-[4-(1,2-Dimethyl-3-oxo-5- | ¹H NMR (400 MHz, DMSO-d6): 9.845 (1H, | 373.7 |
| | quinoxalin-6-yl-2,3-dihydro-1H- | s), 9.022 (1H, m), 9.002 (1H, m), 8.184 (1H, | |
| | pyrazol-4-yl)-phenyl]-acetamide | d, J = 9.1 Hz), 8.077 (1H, m), 7.716 (1H, d, | |
| | | J = 8.7 Hz), 7.358 (2H, d. J = 8.7 Hz), 7.149 (2H, | |
| | | d, J = 8.7 Hz), 3.423 (3H, s), 3.171 (3H, s). | |
| | | 1.971 (3H, s). | |
| 31 | 4-(3-Hydroxy-phenyl)-1,2-dimethyl- | ¹H NMR (400 MHz, DMSO-d6): 9.027 (1H, | 332.75 |
| | 5-quinoxalin-6-yl-1,2-dihydro- | m), 9.013 (1H, m), 8.187 (1H, d, J = 8.7 Hz), | |
| | pyrazol-3-one | 8.093 (1H, d, J = 1.9 Hz), 7.724 (1H, d of d, | |
| | | J = 8.8 Hz, 1.8 Hz), 6.916 (1H, d of d, J = 7.9 Hz, | |
| | | 7.9 Hz), 6.774 (1H, m), 6.581 (1H, d, | |
| | | J = 7.9 Hz), 6.490 (1H, d, 7.8 Hz), 3.430 (3H, s), | |
| | | 3.182 (3H, s). | |
| 32 | 4-(4-Hydroxy-phenyl)-1,2-dimethyl- | ¹H NMR (400 MHz, DMSO-d6): 9.016 (1H, | 332.72 |
| | 5-quinoxalin-6-yl-1,2-dihydro- | d, J = 1.8 Hz), 8.998 (1H, d, J = 1.8 Hz), | |
| | pyrazol-3-one | 8.167 (1H, d, J = 8.7 Hz), 8.063 (1H, d, J = 1.8 Hz), | |
| | | 7.709 (1H, d of d, J = 8.6 Hz, 2.0 Hz), 7.039 (2H, | |
| | | d, J = 9.0 Hz), 6.572 (2H, d, J = 8.8 Hz), | |
| | | 3.409 (3H, s), 3.132 (3H, s). | |
| 33 | 4-Furan-2-yl-1,2-dimethyl-5- | ¹H NMR (400 MHz, DMSO-d6): | 306.77 |
| | quinoxalin-6-yl-1,2-dihydro-pyrazol- | 9.079-9.018 (2H, m), 8.230 (1H, d, 8.8 Hz), 8.199 (1H, m), | |
| | 3-one | 7.880 (1H, d of d, J = 8.7 Hz, 1.8 Hz), 7.300 (1H, | |
| | | m), 6.740 (1H, d, 3.2 Hz), 6.415 (1H, m), | |
| | | 3.417 (3H, s), 3.216 (3H, s). | |
| 34 | 4-(3-Bromo-phenyl)-1,2-dimethyl-5- | ¹H NMR (400 MHz, DMSO-d6): | 394.6 |
| | quinoxalin-6-yl-1,2-dihydro-pyrazol- | 9.062-9.017 (2H, m), 8.224 (1H, d, J = 8.6 Hz), 8.152 (1H, | 396.89 |
| | 3-one | m), 7.763 (1H, d of d, J = 8.6 Hz, 1.8 Hz, | |
| | | 7.646 (1H, m), 7.260 (1H, m), 7.082-6.998 (2H, m), | |
| | | 3.453 (3H, m), 3.252 (3H, m). | |
| 35 | 4-Benzo[b]thiophen-2-yl-1,2- | ¹H NMR (400 MHz, DMSO-d6): 9.099 (1H, | 372.74 |
| | dimethyl-5-quinoxalin-6-yl-1,2- | d, J = 1.8 Hz), 9.070 (1H, d, J = 1.9 Hz), | |
| | dihydro-pyrazol-3-one | 8.324 (1H, d, J = 8.3 Hz), 8.320 (1H, d, J = 2.0 Hz), | |
| | | 7.947 (1H, d of d, J = 8.7 Hz, 1.8 Hz), 7.674 (1H, | |
| | | d, J = 7.8 Hz), 7.621 (1H, d, J = 7.8 Hz), | |
| | | 7.589 (1H, s), 7.209 (1H, d of d of d, J = 7.8 Hz, | |
| | | 7.8 Hz, 1.2 Hz), 7.133 (1H, d of d of d, | |
| | | J = 7.6 Hz, 7.6 Hz, 1.2 Hz), 3.496 (3H, s), | |
| | | 3.285 (3H, s). | |
| 36 | 4-(1H-Indol-5-yl)-1,2-dimethyl-5- | ¹H NMR (400 MHz, DMSO-d6): 11.009 (1H, | 355.83 |
| | quinoxalin-6-yl-1,2-dihydro-pyrazol- | s (br)), 8.999 (1H, d, J = 1.8 Hz), 8.974 (1H, d, | |
| | 3-one | J = 1.8 Hz), 8.125 (d, J = 8.8 Hz), 8.079 (1H, d, | |
| | | J = 1.8 Hz), 7.707 (1H, d of d, J = 8.5 Hz, 2.0 Hz), | |
| | | 7.532 (1H, s), 7.251 (1H, d of d, J = 2.8 Hz, | |
| | | 2.8 Hz), 7.164 (1H, d, J = 8.4 Hz), 6.823 (1H, d | |
| | | of d, J = 8.3 Hz, 1.5 Hz), 6.283 (1H, m), | |
| | | 3.456 (3H, s), 3.176 (3H, s). | |
| 37 | 4-(1H-Indazol-6-yl)-1,2-dimethyl-5- | ¹H NMR (400 MHz, DMSO-d6): | 356.74 |
| | quinoxalin-6-yl-1,2-dihydro-pyrazol- | 13.391-12.355 (1H, s (br)), 9.032 (1H, d, J = 1.8 Hz), | |
| | 3-one | 9.004 (1H, d, J = 1.7 Hz), 8.186 (1H, d, | |
| | | J = 8.6 Hz), 8.140 (1H, d, J = 1.9 Hz), 7.917 (1H, | |
| | | s), 7.752 (1H, d of d, J = 8.6 Hz, 1.9 Hz), | |
| | | 7.617 (1H, s), 7.462 (1H, d, J = 8.6 Hz), 6.820 (1H, d | |
| | | of d, J = 8.6 Hz, 1.3 Hz), 3.472 (3H, s), | |
| | | 3.232 (3H, s). | |
| 38 | 1,2-Dimethyl-4,5-di-quinoxalin-6-yl- | ¹H NMR (400 MHz, DMSO-d6): 9.056 (1H, | 368.62 |
| | 1,2-dihydro-pyrazol-3-one | m), 9.025 (1H, d, J = 1.6 Hz), 8.776 (2H, m), | |
| | | 8.249-8.207 (2H, m), 8.031 (1H, m), | |
| | | 7.838 (1H, m), 7.817 (1H, m), 7.697 (1H, d, | |
| | | J = 8.9 Hz), 3.522 (3H, s), 3.336 (3H, s). | |
| 39 | 1-[3-(1,2-Dimethyl-3-oxo-5- | ¹H NMR (400 MHz, DMSO-d6): 9.038 (1H, | 441.91 |
| | quinoxalin-6-yl-2,3-dihydro-1H- | d, J = 1.9 Hz), 9.013 (1H, d, J = 1.6 Hz), | |
| | pyrazol-4-yl)-benzoyl]-piperidin-4- | 8.209 (1H, d, J = 8.7 Hz), 8.128 (1H, d, J = 1.6 Hz), | |
| | one | 7.774 (1H, d of d, J = 8.7 Hz, 1.9 Hz), 7.381 (1H, | |
| | | m), 7.319-7.194 (3H, m), 3.842-3.188 (4H, | |
| | | m), 3.456 (3H, s), 3.241 (3H, s), | |
| | | 2.442-1.920 (4H, m). | |
| 40 | 1,2-Dimethyl-5-quinoxalin-6-yl-4- | ¹H NMR (400 MHz, DMSO-d6): 9.046 (1H, | 445.91 |
| | [3-(thiomorpholine-4-carbonyl)- | d, J = 1.8 Hz), 9.024 (1H, d, J = 1.8 Hz), | |
| | phenyl]-1,2-dihydro-pyrazol-3-one | 8.224 (1H, d, J = 8.4 Hz), 8.136 (1H, d, J = 1.6 Hz), | |
| | | 7.776 (1H, d of d, J = 8.7 Hz, 1.8 Hz), 7.304 (1H, | |
| | | d, J = 8.0 Hz), 7.273-7.217 (2H, m), 7.112 (1H, | |
| | | d, J = 7.8 Hz), 3.905-3.043 (4H, m), 3.458 (3H, | |
| | | s), 3.244 (3H, s), 2.511-2.098 (4H, m). | |
| 41 | N-(2-Dimethylamino-ethyl)-3-(1,2- | ¹H NMR (400 MHz, DMSO-d6): 9.593 (1H, s | 430.93 |
| | dimethyl-3-oxo-5-quinoxalin-6-yl- | (br)), 9.028 (1H, m), 9.009 (1H, m), | |
| | 2,3-dihydro-1H-pyrazol-4-yl)- | 8.603 (1H, m), 8.190 (1H, d, J = 8.6 Hz), 8.101 (1H, s | |
| | benzamide | (br)), 7.939 (1H, s), 7.729 (1H, d of d, | |
| | | J = 8.6 Hz, 1.7 Hz), 7.608 (1H, m), | |
| | | 7.270-7.189 (2H, m), 3.573-3.496 (2H, m), 3.464 (3H, s), | |
| | | 3.275-3.165 (2H, m), 3.245 (3H, s), | |
| | | 2.804 (3H, s), 2.792 (3H, s). | |
| 42 | [3-(1,2-Dimethyl-3-oxo-5- | ¹H NMR (400 MHz, DMSO-d6): 9.022 (1H, | 355.71 |
| | quinoxalin-6-yl-2,3-dihydro-1H- | d, J = 1.8 Hz), 9.001 (1H, d, J = 1.8 Hz), | |
| | pyrazol-4-yl)-phenyl]-acetonitrile | 8.189 (1H, d, J = 8.6 Hz), 8.108 (1H, d, J = 1.6 Hz), | |
| | | 7.731 (1H, d of d, J = 8.7 Hz, 1.8 Hz), 7.342 (1H, | |
| | | s (br)), 7.183-7.084 (2H, m), 7.050 (1H, d, | |
| | | J = 6.0 Hz), 3.869 (2H, s), 3.452 (3H, s), | |
| | | 3.231 (3H, s). | |
| 43 | N-[4-(1,2-Dimethyl-3-oxo-5- | ¹H NMR (400 MHz, DMSO-d6): 9.027 (1H, | 423.84 |
| | quinoxalin-6-yl-2,3-dihydro-1H- | d, J = 1.7 Hz), 9.008 (1H, d, J = 1.9 Hz), | |
| | pyrazol-4-yl)-benzyl]- | 8.193 (1H, d, J = 8.7 Hz), 8.090 (1H, d, J = 1.9 Hz), | |
| | methanesulfonamide | 7.731 (1H, d of d, J = 8.5 Hz, 1.9 Hz), 7.443 (1H, | |
| | | d of d, J = 6.2 Hz, 6.2 Hz), 7.220 (2H, d, | |
| | | J = 8.3 Hz), 7.134 (2H, d, J = 8.3 Hz), 4.043 (2H, | |
| | | d, J = 6.0 Hz), 3.440 (3H, s), 3.203 (3H, s), | |
| | | 2.789 (3H, s). | |
| 44 | 1,2-Dimethyl-4-[3-(morpholine-4- | ¹H NMR (400 MHz, DMSO-d6): 9.046 (1H, | 429.9 |
| | carbonyl)-phenyl]-5-quinoxalin-6-yl- | d, J = 1.6 Hz), 9.022 (1H, d, J = 1.9 Hz), | |
| | 1,2-dihydro-pyrazol-3-one | 8.214 (1H, d, J = 8.6 Hz), 8.131 (1H, d, J = 1.9 Hz), | |
| | | 7.769 (1H, d of d, J = 8.6 Hz, 2.1 Hz), 7.338 (1H, | |
| | | d of d of d, J = 7.6 Hz, 1.4 Hz, 1.4 Hz), | |
| | | 7.268 (1H, d of d, J = 7.6 Hz, 7.6 Hz), 7.220 (1H, m), | |
| | | 7.142 (1H, d of d of d, J = 7.6 Hz, 1.4 Hz, | |
| | | 1.4 Hz), 3.686-2.808 (8H, m), 3.466 (3H, s), | |
| | | 3.251 (3H, s). | |
| 45 | N-[3-(1,2-Dimethyl-3-oxo-5- | ¹H NMR (400 MHz, DMSO-d6): 9.020 (1H, | 423.85 |
| | quinoxalin-6-yl-2,3-dihydro-1H- | d, J = 1.8 Hz), 8.998 (1H, d, J = 1.8 Hz), | |
| | pyrazol-4-yl)-benzyl]- | 8.178 (1H, d, J = 8.6 Hz), 8.086 (1H, d, J = 1.8 Hz), | |
| | methanesulfonamide | 7.720 (1H, d of d, J = 8.8 Hz, 1.9 Hz), 7.426 (1H, | |
| | | m), 7.378 (1H, s (br)), 7.142-7.061 (2H, m), | |
| | | 7.002 (1H, d, J = 6.6 Hz), 3.981 (2H, d, | |
| | | J = 5.7 Hz), 3.452 (3H, s), 3.217 (3H, s), | |
| | | 2.703 (3H, s). | |
| 46 | 3-(1,2-Dimethyl-3-oxo-5- | ¹H NMR (400 MHz, DMSO-d6): 12.624 (1H, | 442.83 |
| | quinoxalin-6-yl-2,3-dihydro-1H- | s (br)), 9.030 (1H, d, J = 1.7 Hz), 9.012 (1H, d, | |
| | pyrazol-4-yl)-N-thiazol-2-yl- | J = 1.7 Hz), 8.208 (1H, d, J = 8.5 Hz), 8.143 (1H, | |
| | benzamide | d, J = 1.5 Hz), 8.110 (1H, m), 8.103-7.219 (3H, | |
| | | obscured), 7.842 (1H, d, J = 7.4 Hz), 7.765 (1H, | |
| | | d, J = 8.5 Hz, 2.0 Hz), 7.307 (1H, m). | |
| 47 | 1,2-Dimethyl-4-[2-methyl-5- | ¹H NMR (400 MHz, DMSO-d6): 8.973 (1H, | 479.99 |
| | (morpholine-4-sulfonyl)-phenyl]-5- | d, J = 1.7 Hz), 8.941 (1H, d, J = 1.7 Hz), | |
| | quinoxalin-6-yl-1,2-dihydro-pyrazol- | 8.148 (1H, d, J = 8.5 Hz), 7.889 (1H, d, J = 1.7 Hz), | |
| | 3-one | 7.732 (1H, d of d, J = 8.5 Hz, 1.7 Hz), 7.531 (1H, | |
| | | d, J = 8.3 Hz), 7.442 (1H, d of d, J = 8.0 Hz, | |
| | | 2.0 Hz), 7.002 (1H, d, J = 2.0 Hz), 3.467 (3H, s), | |
| | | 3.314 (3H, s), 3.300-3.288 (4H, m), | |
| | | 2.454 (3H, s), 2.224-1.922 (4H, m). | |
| 48 | 4-(3-Dimethylaminomethyl-phenyl)- | ¹H NMR (400 MHz, DMSO-d6): 9.583 (1H, s | 373.86 |
| | 1,2-dimethyl-5-quinoxalin-6-yl-1,2- | (br)), 9.037 (1H, d, J = 1.8 Hz), 9.009 (1H, d, | |
| | dihydro-pyrazol-3-one | J = 1.6 Hz), 8.191 (1H, d, J = 8.6 Hz), 8.103 (1H, | |
| | | d, J = 1.8 Hz), 7.748 (1H, d of d, J = 8.8 Hz, | |
| | | 2.0 Hz), 7.351 (1H, s (br)), 7.329-7.278 (2H, | |
| | | m), 7.278-7.198 (1H, m), 4.079 (2H, d, | |
| | | J = 5.0 Hz), 3.454 (3H, s), 3.243 (3H, s), | |
| | | 2.533-2.458 (6H, m). | |
| 49 | [3-(1,2-Dimethyl-3-oxo-5- | ¹H NMR (400 MHz, DMSO-d6): 9.023 (1H, | 374.81 |
| | quinoxalin-6-yl-2,3-dihydro-1H- | d, J = 1.8 Hz), 9.002 (1H, d, J = 1.8 Hz), | |
| | pyrazol-4-yl)-phenyl]-acetic acid | 8.170 (1H, d, 8.7 Hz), 8.097 (1H, d, J = 1.8 Hz), | |
| | | 7.717 (1H, d of d, J = 8.7 Hz, 1.9 Hz), | |
| | | 7.325-7.197 (2H, m), 7.103-7.047 (1H, m), | |
| | | 7.037-6.972 (2H, m), 3.440 (3H, s), 3.364 (2H, s), | |
| | | 3.200 (3H, s). | |
| 50 | 4-(2-tert-Butoxymethyl-phenyl)-1,2- | ¹H NMR (400 MHz, DMSO-d6): 8.964 (1H, | 402.93 |
| | dimethyl-5-quinoxalin-6-yl-1,2- | m), 8.944 (1H, m), 8.086 (1H, d, J = 8.6 Hz), | |
| | dihydro-pyrazol-3-one | 8.057 (1H, m), 7.742 (1H, d of d, J = 8.6 Hz, | |
| | | 1.9 Hz), 7.393 (1H, d, J = 7.7 Hz), 7.201 (1H, d | |
| | | of d, J = 7.5 Hz, 7.5 Hz), 7.041 (1H, d of d, | |
| | | J = 7.5 Hz, 7.5 Hz), 6.818 (1H, d, J = 7.5 Hz), | |
| | | 4.353 (2H, m), 3.442 (3H, s), 3.237 (3H, s), | |
| | | 1.084 (9H, s). | |
| 51 | 4-(2-Hydroxy-phenyl)-1,2-dimethyl- | ¹H NMR (400 MHz, DMSO-d6): 9.027 (1H, | 332.74 |
| | 5-quinoxalin-6-yl-1,2-dihydro- | d, J = 1.8 Hz), 9.006 (1H, d, J = 1.8 Hz), | |
| | pyrazol-3-one | 8.185 (1H, d, J = 8.7 Hz), 8.109 (1H, d, J = 2.0 Hz), | |
| | | 7.725 (1H, d of d, J = 8.7 Hz, 2.0 Hz), 6.978 (1H, | |
| | | d of d, J = 7.5 Hz, 7.5 Hz), 6.755 (1H, d, | |
| | | J = 8.0 Hz), 6.610 (1H, d of d, J = 7.9 Hz, 1.8 Hz), | |
| | | 6.457 (1H, d of d, J = 7.5 Hz, 7.5 Hz), 3.564 (3H, | |
| | | s), 3.397 (3H, s). | |
| 52 | 4-(1,2-Dimethyl-3-oxo-5- | ¹H NMR (400 MHz, DMSO-d6): 9.049 (1H, | 395.71 |
| | quinoxalin-6-yl-2,3-dihydro-1H- | d, J = 1.8 Hz), 9.027 (1H, d, J = 1.8 Hz), | |
| | pyrazol-4-yl)-benzenesulfonamide | 8.232 (1H, d, J = 8.7 Hz), 8.152 (1H, d, J = 1.9 Hz), | |
| | | 7.762 (1H, d of d, J = 8.7 Hz, 1.9 Hz), 7.580 (2H, | |
| | | d, J = 8.6 Hz), 7.410 (2H, d, J = 8.6 Hz), | |
| | | 7.202 (2H, s (br)), 3.472 (3H, s), 3.281 (3H, s). | |
| 53 | 4-Benzo[1,2,5]oxadiazol-5-yl-1,2- | ¹H NMR (400 MHz, DMSO-d6): 9.075 (1H, | 358.8 |
| | dimethyl-5-quinoxalin-6-yl-1,2- | d, J = 1.8 Hz), 9.049 (1H, d, J = 1.9 Hz), | |
| | dihydro-pyrazol-3-one | 8.267 (1H, d, J = 7.3 Hz), 8.254 (1H, s), 7.987 (1H, | |
| | | m), 7.852 (1H, d of d, J = 8.6 Hz, 2.1 Hz), | |
| | | 7.746 (1H, d of d, J = 9.7 Hz, 1.1 Hz), 7.289 (1H, d of | |
| | | d, J = 9.4 Hz, 1.3 Hz), 3.522 (3H, s), 3.372 (3H, | |
| | | s). | |
| 54 | 1′-Benzyl-1,2-dimethyl-5- | ¹H NMR (400 MHz, DMSO-d6): 9.052 (1H, | 396.63 |
| | quinoxalin-6-yl-1,2-dihydro-1′H- | m), 9.042 (1H, m), 8.248 (1H, d, J = 8.5 Hz), | |
| | [4,4′]bipyrazolyl-3-one | 8.170 (1H, d, J = 1.8 Hz), 7.880-7.821 (2H, m), | |
| | | 7.305-7.197 (3H, m), 7.155-7.086 (3H, m), | |
| | | 5.221 (2H, s), 3.400 (3H, s), 3.116 (3H, s). | |
| 55 | 4-(3-Methoxymethyl-phenyl)-1,2- | ¹H NMR (400 MHz, DMSO-d6): 9.024 (1H, | 360.85 |
| | dimethyl-5-quinoxalin-6-yl-1,2- | m), 9.003 (1H, m), 8.186 (1H, d, J = 8.6 Hz), | |
| | dihydro-pyrazol-3-one | 8.094 (1H, d, J = 1.6 Hz), 7.735 (1H, d of d, | |
| | | J = 8.5 Hz, 1.8 Hz), 7.230 (1H, m), | |
| | | 7.153-7.093 (2H, m), 7.058-6.999 (1H, m), 4.203 (2H, s), | |
| | | 3.446 (3H, s), 3.216 (3H, s), 3.008 (3H, s). | |
| 56 | 4-(2-Hydroxymethyl-phenyl)-1,2- | ¹H NMR (400 MHz, DMSO-d6): 8.975 (1H, | 346.69 |
| | dimethyl-5-quinoxalin-6-yl-1,2- | m), 8.955 (1H, m), 8.086 (1H, d, J = 8.8 Hz), | |
| | dihydro-pyrazol-3-one | 7.971 (1H, s), 7.638 (1H, d, J = 8.6 Hz), | |
| | | 7.455 (1H, d, J = 7.7 Hz), 7.208 (1H, d of d, J = 7.4 Hz, | |
| | | 7.4 Hz), 6.987 (1H, d of d, J = 7.2 Hz, 7.2 Hz), | |
| | | 6.761 (1H, d, J = 7.9 Hz), 3.466 (3H, s), | |
| | | 3.288 (3H, s). | |
| 57 | 4-(3-Benzo[1,2,5]thiadiazol-5-yl-5- | ¹H NMR (300 MHz, CDCl₃): d 3.14 (s, 3H), | 316.83 |
| | methoxy-pyrazol-1-yl)-benzoic acid | 3.52 (s, 3H), 7.16-7.33 (m, 3H), 7.32 (d, J = 5.4 Hz, | |
| | methyl ester | 2H), 7.64 (d, J = 6.3 Hz, 1H), 8.60 (d, | |
| | | J = 6.6 Hz, 1H), 8.15 (s, 1H), 8.90 (d, J = 3 Hz, | |
| | | 2H) | |
| 58 | 1,2-Dimethyl-4-phenyl-5- | ¹H NMR (CDCl₃, 300 MHz) d 2.06 (s, 3H), | 331.7 |
| | quinoxalin-6-yl-1,2-dihydro-pyrazol- | 3.27 (s, 3H), 3.58 (s, 3H), 6.88 (d, J = 8 Hz, 1H), | |
| | 3-one: | 7.55 (t, J = 8 Hz, 1H), 7.79 (dd, J = 8 Hz, J = 2 Hz, | |
| | | 1H), 7.94 (d, J = 8 Hz, 1H), 8.12 (d, J = 8 Hz, 1H), | |
| | | 8.22 (d, J = 2 Hz, 1H), 8.92 (q, J = 2 Hz, 2H); | |
| 59 | 1,2-Dimethyl-4-(6-methyl-pyridin-2- | ¹H NMR (400 MHz, DMSO-d6): 9.047 (1H, | 331.82 |
| | yl)-5-quinoxalin-6-yl-1,2-dihydro- | m), 9.025 (1H, m), 8.219 (1H, d, J = 8.8 Hz), | |
| | pyrazol-3-one | 8.130 (1H, m), 7.747 (1H, d, J = 8.6 Hz), | |
| | | 7.417 (1H, s), 7.156 (1H, d of d, J = 7.8 Hz, 7.8 Hz), | |
| | | 6.954 (1H, d, J = 8.0 Hz), 3.462 (3H, s), | |
| | | 3.248 (3H, s). | |
| 60 | 4-(3-Aminophenyl)-1,2-dihydro-1,2- | ¹H NMR (400 MHz, DMSO-d6): 9.043 (1H, | 384.87 |
| | dimethyl-5-(quinoxalin-7-yl)pyrazol- | m), 9.019 (1H, m), 8.240-8.187 (2H, m), | |
| | 3-one | 8.164 (1H, m), 8.025 (1H, m), 7.768 (1H, d, | |
| | | J = 8.6 Hz), 7.561 (1H, d, J = 9.0 Hz), 7.424 (1H, | |
| | | d, J = 8.7 Hz), 6.225 (1H, d, J = 6.1 Hz), | |
| | | 3.480 (3H, s), 3.273 (3H, s). | |
| 61 | 1,2-Dihydro-1,2-dimethyl-4-(4-oxo- | ¹H NMR (400 MHz, DMSO-d6): 9.055 (1H, | 351.64 |
| | 4H-chromen-6-yl)-5-(quinoxalin-7- | d, J = 1.9 Hz), 9.033 (1H, d, J = 1.8 Hz), | |
| | yl)pyrazol-3-one | 8.243 (1H, d, J = 8.6 Hz), 8.216 (1H, d, J = 2.4 Hz), | |
| | | 8.184 (1H, d, J = 1.8 Hz), 7.793 (1H, d of d, | |
| | | J = 8.6 Hz, 2.1 Hz), 7.686 (1H, d of d, J = 8.5 Hz, | |
| | | 2.6 Hz), 7.330 (1H, d, J = 8.3 Hz), 3.479 (3H, s), | |
| | | 3.309 (3H, s). | |
| 62 | 4-(6-Chloropyridin-3-yl)-1,2- | ¹H NMR (400 MHz, DMSO-d6): 9.043 (1H, | 345.74 |
| | dihydro-1,2-dimethyl-5-(quinoxalin- | d, J = 1.8 Hz), 9.023 (1H, d, J = 1.8 Hz), | |
| | 7-yl)pyrazol-3-one | 8.204 (1H, d, J = 8.5 Hz), 8.123 (1H, d, J = 1.8 Hz), | |
| | | 7.729 (1H, d of d, J = 8.5 Hz, 1.8 Hz), 7.417 (1H, | |
| | | m), 7.042 (1H, d, J = 8.1 Hz), 6.931 (1H, d, | |
| | | J = 8.3 Hz), 3.452 (3H, s), 3.232 (3H, s), | |
| | | 2.171 (3H, s). | |
| 63 | 4-(3-Amino-4-methylphenyl)-1,2- | ¹H NMR (400 MHz, DMSO-d6): 9.026 (1H, | 365.61 |
| | dihydro-1,2-dimethyl-5-(quinoxalin- | d, J = 1.9 Hz), 9.010 (1H, d, J = 1.9 Hz), | |
| | 7-yl)pyrazol-3-one | 8.182 (1H, d, J = 8.7 Hz), 8.092 (1H, d, J = 1.7 Hz), | |
| | | 7.710 (1H, d of d, J = 8.7 Hz, 2.1 Hz), 6.956 (1H, | |
| | | d, J = 8.3 Hz), 6.922 (1H, d, J = 2.1 Hz), | |
| | | 6.316 (1H, d of d, J = 8.3 Hz, 2.1 Hz), 3.433 (3H, s), | |
| | | 3.194 (3H, s). | |
| 64 | 4-(3-Amino-4-chlorophenyl)-1,2- | ¹H NMR (400 MHz, DMSO-d6): 9.035 (1H, | 345.8 |
| | dihydro-1,2-dimethyl-5-(quinoxalin- | d, J = 1.9 Hz), 9.011 (1H, d, J = 1.8 Hz), | |
| | 7-yl)pyrazol-3-one | 8.195 (1H, d, J = 8.6 Hz), 8.173-8.049 (4H, m), | |
| | | 7.717 (1H, d of d, J = 8.5 Hz, 1.9 Hz), 7.582 (1H, m), | |
| | | 7.241-7.123 (2H, m), 7.002 (1H, d, J = 7.5 Hz), | |
| | | 3.901 (2H, m), 3.457 (3H, s), 3.232 (3H, s). | |
| 65 | 4-(3-(Aminomethyl)phenyl)-1,2- | ¹H NMR (400 MHz, DMSO-d6): 9.049 (1H, | 394.88 |
| | dihydro-1,2-dimethyl-5-(quinoxalin- | m), 9.023 (1H, m), 8.230 (1H, d, J = 8.7 Hz), | |
| | 7-yl)pyrazol-3-one | 8.174 (1H, m), 7.855 (1H, m), 7.776 (1H, d, | |
| | | J = 8.7 Hz), 7.610 (1H, d, J = 8.1 Hz), 7.527 (1H, | |
| | | d, J = 7.7 Hz), 7.407 (1H, d of d, J = 7.9 Hz, | |
| | | 7.9 Hz), 3.480 (3H, s), 3.300 (3H, s), | |
| | | 2.977 (3H, s). | |
| 66 | 1,2-Dihydro-1,2-dimethyl-4-(3- | ¹H NMR (400 MHz, DMSO-d6): | 395.82 |
| | (methylsulfonyl)phenyl)-5- | 9.101-9.002 (2H, m), 8.205 (1H, d, J = 8.5 Hz), 8.136 (1H, | |
| | (quinoxalin-7-yl)pyrazol-3-one | m), 7.926 (1H, m), 7.738 (1H, d, J = 8.5 Hz), | |
| | | 7.541 (1H, m), 7.370-7.184 (4H, m), | |
| | | 3.467 (3H, s), 3.264 (3H, s). | |
| 67 | 1,2-Dihydro-1,2-dimethyl-4-(3- | ¹H NMR (400 MHz, DMSO-d6): | 346.75 |
| | (aminosulfonyl)phenyl)-5- | 9.098-8.996 (2H, m), 8.198 (1H, d, J = 8.7 Hz), 8.117 (1H, | |
| | (quinoxalin-7-yl)pyrazol-3-one | m), 7.749 (1H, d, J = 8.5 Hz), 7.040 (1H, d of d, | |
| | | J = 8.0 Hz, 8.0 Hz), 6.898 (1H, m), 6.744 (1H, d, | |
| | | J = 7.8 Hz), 6.669 (1H, d, J = 8.9 Hz), 3.510 (3H, | |
| | | s), 3.441 (3H, s), 3.207 (3H, s). | |
| 68 | 1,2-Dihydro-4-(3-methoxyphenyl)- | ¹H NMR (400 MHz, DMSO-d6): 8.990 (1H, | 355.85 |
| | 1,2-dimethyl-5-(quinoxalin-7- | d, J = 1.7 Hz), 8.967 (1H, d, J = 1.6 Hz), | |
| | yl)pyrazol-3-one | 8.134 (1H, d, J = 8.6 Hz), 8.050 (1H, d, J = 1.7 Hz), | |
| | | 7.705 (1H, d of d, J = 8.6 Hz, 1.7 Hz), 7.438 (1H, | |
| | | d, J = 7.7 Hz), 7.237 (1H, d of d, J = 7.5 Hz, | |
| | | 7.5 Hz), 7.035 (1H, d of d, J = 7.5 Hz, 7.5 Hz), | |
| | | 6.773 (1H, d, J = 7.5 Hz), 4.187 (2H, s (br)), | |
| | | 3.490 (3H, s), 3.325 (3H, s). | |
| 69 | 2-(2-(2,3-Dihydro-1,2-dimethyl-3- | ¹H NMR (400 MHz, DMSO-d6): 9.805 (1H, | 373.81 |
| | oxo-5-(quinoxalin-7-yl)-1H-pyrazol- | s (br)), 9.021 (1H, d, J = 1.8 Hz), 9.002 (1H, d, | |
| | 4-yl)phenyl)acetonitrile | J = 2.1 Hz), 8.173 (1H, d, J = 8.7 Hz), 8.088 (1H, | |
| | | d, J = 1.5 Hz), 7.709 (1H, d of d, J = 8.7 Hz, | |
| | | 2.1 Hz), 7.538 (1H, m), 7.458 (1H, d, | |
| | | J = 8.5 Hz), 7.026 (1H, d of d, J = 8.0 Hz, 8.0 Hz), | |
| | | 6.730 (1H, d, J = 8.0 Hz), 3.443 (3H, s), | |
| | | 3.205 (3H, s), 1.924 (3H, s). | |
| 70 | N-(3-(2,3-Dihydro-1,2-dimethyl-3- | ¹H NMR (400 MHz, DMSO-d6): 9.044 (1H, | 331.79 |
| | oxo-5-(quinoxalin-7-yl)-1H-pyrazol- | d, J = 2.0 Hz), 9.016 (1H, d, J = 2.0 Hz), | |
| | 4-yl)phenyl)acetamide | 8.235 (1H, d, J = 8.7 Hz), 8.205 (1H, d, J = 1.8 Hz), | |
| | | 7.795 (1H, d of d, J = 8.5 Hz, 2.0 Hz), 7.474 (1H, | |
| | | d, J = 8.0 Hz), 7.259 (1H, d of d of d, J = 7.8 Hz, | |
| | | 7.8 Hz, 1.4 Hz), 6.991 (1H, d of d of d, | |
| | | J = 7.6 Hz, 7.6 Hz, 1.1 Hz), 6.661 (1H, d of d, | |
| | | J = 7.8 Hz, 1.3 Hz), 3.646 (3H, s), 3.580 (3H, s). | |
| 71 | 4-(2-Aminophenyl)-1,2-dihydro-1,2- | ¹H NMR (400 MHz, DMSO-d6): 9.049 (1H, | 376.83 |
| | dimethyl-5-(quinoxalin-7-yl)pyrazol- | d, J = 1.7 Hz), 9.030 (1H, d, J = 1.7 Hz), | |
| | 3-one | 8.222 (1H, d, J = 8.8 Hz), 8.164 (1H, d, J = 1.7 Hz), | |
| | | 7.761 (1H, d of d, J = 8.5 Hz, 1.7 Hz), 7.290 (1H, | |
| | | m), 7.143 (1H, d of d, J = 2.1 Hz, 2.1 Hz), | |
| | | 6.940 (1H, m), 3.465 (3H, s), 3.265 (3H, s). | |
| 72 | 4-(3-Amino-5-nitrophenyl)-1,2- | ¹H NMR (400 MHz, DMSO-d6): 9.047 (1H, | 367.86 |
| | dihydro-1,2-dimethyl-5-(quinoxalin- | m), 8.973 (1H, d, J = 1.6 Hz), 8.941 (1H, d, | |
| | 7-yl)pyrazol-3-one | J = 1.6 Hz), 8.858 (1H, d, J = 8.1 Hz), | |
| | | 8.108-8.035 (3H, m), 7.843 (1H, d of d, J = 8.4 Hz, 4.9 Hz), | |
| | | 7.706 (1H, d of d, J = 8.6 Hz, 1.9 Hz), | |
| | | 7.634-7.556 (2H, m), 3.632 (3H, s), 3.512 (3H, s). | |
| 73 | 1,2-Dihydro-1,2-dimethyl-4- | ¹H NMR (400 MHz, DMSO-d6): 9.052 (1H, | 389.86 |
| | (quinolin-8-yl)-5-(quinoxalin-7- | d, J = 1.7 Hz), 9.031 (1H, d, J = 1.7 Hz), | |
| | yl)pyrazol-3-one | 8.220 (1H, d, J = 8.5 Hz), 8.148 (1H, d, J = 1.7 Hz), | |
| | | 7.750 (1H, d of d, J = 8.7 Hz, 1.9 Hz), 7.346 (1H, | |
| | | m), 7.226 (1H, m), 7.211 (1H, m), 3.619 (3H, | |
| | | s), 3.462 (3H, s), 3.260 (3H, s). | |
| 74 | Methyl 3-amino-5-(2,3-dihydro-1,2- | ¹H NMR (400 MHz, DMSO-d6): 9.082 (1H, | 317.61 |
| | dimethyl-3-oxo-5-(quinoxalin-7-yl)- | d, J = 1.7 Hz), 9.058 (1H, d, J = 1.7 Hz), | |
| | 1H-pyrazol-4-yl)benzoate | 8.929 (1H, d, J = 1.9 Hz), 8.558 (1H, d, J = 5.3 Hz), | |
| | | 8.293 (1H, d, J = 5.5 Hz), 8.280 (1H, s), | |
| | | 8.037 (1H, d, J = 8.4 Hz), 7.858 (1H, d of d, J = 8.7 Hz, | |
| | | 1.7 Hz), 7.719 (1H, d of d, J = 8.4 Hz, 5.6 Hz), | |
| | | 3.535 (3H, s), 3.422 (3H, s). | |
| 75 | 1,2-Dihydro-1,2-dimethyl-4- | ¹H NMR (400 MHz, DMSO-d6): 9.049 (1H, | 368.62 |
| | (pyridin-3-yl)-5-(quinoxalin-7- | d, J = 1.7 Hz), 9.031 (1H, d, J = 1.8 Hz), | |
| | yl)pyrazol-3-one | 8.229 (1H, d, J = 8.6 Hz), 8.166 (1H, d, J = 1.8 Hz), | |
| | | 7.769 (1H, d of d, J = 8.6 Hz, 1.8 Hz), 7.635 (1H, | |
| | | d of d, J = 7.5 Hz, 2.2 Hz), 7.160 (1H, d of d, | |
| | | 9.0 Hz, 9.0 Hz), 7.008 (1H, d of d of d, | |
| | | J = 8.6 Hz, 4.6 Hz, 2.2 Hz), 3.464 (3H, s), | |
| | | 3.260 (3H, s). | |
| 76 | 4-(3-Chloro-4-fluorophenyl)-1,2- | ¹H NMR (400 MHz, DMSO-d6): 9.036 (1H, | 387.87 |
| | dihydro-1,2-dimethyl-5-(quinoxalin- | d, J = 1.7 Hz), 9.011 (1H, d, J = 1.9 Hz), | |
| | 7-yl)pyrazol-3-one | 8.207 (1H, d, J = 8.6 Hz), 8.122 (1H, d, J = 1.7 Hz), | |
| | | 7.774 (1H, d of d, J = 8.6 Hz, 1.9 Hz), | |
| | | 7.304-7.196 (3H, m), 7.115 (1H, d of d of d, | |
| | | J = 6.7 Hz, 1.9 Hz, 1.9 Hz), 3.459 (3H, s), | |
| | | 3.243 (3H, s), 2.817 (3H, s (br)), 2.556 (3H, s (br)). | |
| 77 | 3-(2,3-Dihydro-1,2-dimethyl-3-oxo- | ¹H NMR (400 MHz, DMSO-d6): 9.046 (1H, | 374.86 |
| | 5-(quinoxalin-7-yl)-1H-pyrazol-4- | m), 9.025 (1H, m), 8.215 (1H, d, J = 8.7 Hz), | |
| | yl)-N,N-dimethylbenzamide | 8.150 (1H, m), 7.994 (1H, m), 7.759 (1H, d, | |
| | | J = 8.7 Hz), 7.661 (1H, d, J = 7.4 Hz), 7.419 (1H, | |
| | | d, J = 7.4 Hz), 7.264 (1H, d of d, J = 7.8 Hz, | |
| | | 7.8 Hz), 3.690 (3H, s), 3.469 (3H, s), | |
| | | 3.266 (3H, s). | |
| 78 | Methyl 3-(2,3-dihydro-1,2-dimethyl- | ¹H NMR (300 MHz, DMSO-d6): 9.047 (1H, | 306.82 |
| | 3-oxo-5-(quinoxalin-7-yl)-1H- | d, J = 1.9 Hz), 9.030 (1H, d, J = 1.8 Hz), | |
| | pyrazol-4-yl)benzoate | 8.262 (1H, d, J = 8.7 Hz), 8.178 (1H, d, J = 1.7 Hz), | |
| | | 7.893 (1H, d of d, J = 1.5 Hz, 0.8 Hz), 7.857 (1H, | |
| | | d of d, J = 8.6 Hz, 1.9 Hz), 7.436 (1H, d of d, | |
| | | J = 1.8 Hz, 1.8 Hz), 5.901 (1H, d of d, J = 1.9 Hz, | |
| | | 0.8 Hz), 3.435 (3H, s), 3.174 (3H, s). | |
| 79 | 4-Furan-3-yl-1,2-dimethyl-5- | | |
| | quinoxalin-6-yl-1,2-dihydro-pyrazol- | | |
| | 3-one | | |
| 80 | 5-Benzo[1,2,5]thiadiazol-5-yl-4-(3- | ¹H NMR (300 MHz, (CD₃)₂SO): d 0.89 (t, | 344.6 |
| | bromo-phenyl)-2-(4-hydroxy- | J = 7 Hz, 3H), 2.38 (q, J = 7 Hz, 2H), 3.21 (s, 3H), | |
| | bicyclo[2.2.2]oct-1-ylmethyl)-1- | 3.44 (s, 3H), 6.95 (m, 1H), 7.07 (m, 3H), | |
| | methyl-1,2-dihydro-pyrazol-3-one | 7.75 (dd, J = 8 Hz, J = 2 Hz, 1H), 8.12 (d, J = 2 Hz, 1H), | |
| | | 8.22 (d, J = 8 Hz, 1H), 9.02 (dd, J = 7 Hz, J = 2 Hz, | |
| | | 2H); | |
| 81 | 4-(3-Ethyl-phenyl)-1,2-dimethyl-5- | ¹H NMR (300 MHz, (CD₃)₂SO): d 0.87 (d, | 358.6 |
| | quinoxalin-6-yl-1,2-dihydro-pyrazol- | J = 7 Hz, 6H), 2.61 (m, 1H), 3.22 (s, 3H), | |
| | 3-one | 3.45 (s, 3H), 6.96 (m, 2H), 7.11 (t, J = 7 Hz, 1H), | |
| | | 7.20 (m, 1H), 7.76 (dd, J = 8 Hz, J = 2 Hz, 1H), | |
| | | 8.12 (d, J = 2 Hz, 1H), 8.22 (d, J = 8 Hz, 1H), | |
| | | 9.02 (dd, J = 7 Hz, J = 2 Hz, 2H); | |
| 82 | 4-(3-Isopropyl-phenyl)-1,2- | ¹H NMR (300 MHz, (CD₃)₂SO): d 2.13 (s, | 362.7 |
| | dimethyl-5-quinoxalin-6-yl-1,2- | 3H), 3.24 (s, 3H), 3.46 (s, 3H), 7.04 (m, 3H), | |
| | dihydro-pyrazol-3-one | 7.18 (t, J = 2 Hz, 1H), 7.78 (dd, J = 8 Hz, J = 2 Hz, | |
| | | 1H), 8.14 (d, J = 2 Hz, 1H), 8.23 (d, J = 8 Hz, 1H), | |
| | | 9.02 (dd, J = 7 Hz, J = 2 Hz, 2H); | |
| 83 | 1,2-Dimethyl-4-(3-methylsulfanyl- | ¹H NMR (300 MHz, (CD₃)₂SO): d 3.23 (s, | 342.8 |
| | phenyl)-5-quinoxalin-6-yl-1,2- | 3H), 3.46 (s, 3H), 5.10 (d, J = 11 Hz, 1H), | |
| | dihydro-pyrazol-3-one | 5.46 (d, J = 17 Hz, 1H), 6.55 (dd, J = 17 Hz, J = 11 Hz, | |
| | | 1H), 7.12 (m, 1H), 7.21 (m, 1H), 7.41 (m, 1H), | |
| | | 7.76 (dd, J = 8 Hz, J = 2 Hz, 1H), 8.12 (d, J = 2 Hz, | |
| | | 1H), 8.22 (d, J = 8 Hz, 1H), 9.02 (dd, J = 7 Hz, | |
| | | J = 2 Hz, 2H); | |
| 84 | 1,2-Dimethyl-5-quinoxalin-6-yl-4- | ¹H NMR (300 MHz, (CD₃)₂SO): d 2.47 (s, | 331.6 |
| | (3-vinyl-phenyl)-1,2-dihydro- | 3H), 3.50 (s, 3H), 3.57 (s, 3H), 7.29 (m, 1H), | |
| | pyrazol-3-one | 7.90 (m, 1H), 8.00 (s, 1H), 8.25 (d, J = 7 Hz, | |
| | | 1H), 8.37 (m, 2H), 9.02 (dd, J = 7 Hz, J = 2 Hz, | |
| | | 2H); | |
| 85 | 1,2-Dimethyl-4-(2-methyl-pyridin-4- | ¹H NMR (300 MHz, (CD₃)₂SO): d 2.21 (s, | 331.2 |
| | yl)-5-quinoxalin-6-yl-1,2-dihydro- | 3H), 3.07 (s, 3H), 3.40 (s, 3H), 6.90 (m, 3H), | |
| | pyrazol-3-one | 7.14 (bs, 1H), 7.65 (m, 2H), 8.06 (m, 2H), | |
| | | 8.95 (m, 2H) | |
| 86 | 5-Benzo[1,2,5]thiadiazol-5-yl- | ¹H NMR (300 MHz, DMSO-d6): | 246.48 |
| | 1,2-dimethyl-1,2-dihydro- | 8.283 (1H, m), 8.241 (1H, d, J = 8.9 Hz), | |
| | pyrazol-3-one | 7.826 (1H, d, J = 9.0 Hz), 5.896 (1H, m), | |
| | | 3.383 (3H, s), 3.322 (3H, s) | |
| 87 | 5-Benzo[1,2,5]thiadiazol-5-yl-4- | ¹H NMR (400 MHz, DMSO-d6): | 324.52 |
| | bromo-1,2-dimethyl-1,2-dihydro- | 8.317-8.269 (2H, m), 7.788 (1H, dd, J = 8.8, | |
| | pyrazol-3-one | 1.7 Hz), 3.416 (3H, s), 3.247 (3H, s). | |
| 88 | 5-Benzo[1,2,5]thiadiazol-5-yl-4- | ¹H NMR (300 MHz, DMSO-d6): | 375.17 |
| | (3-chloro-4-fluoro-phenyl)-1,2- | 8.252 (1H, m), 8.221 (1H, d, J = 8.8 Hz), | |
| | dimethyl-1,2-dihydro-pyrazol-3- | 7.688 (1H, dd, J = 7.5, 2.1 Hz), 7.571 (1H, dd = 9.1, | |
| | one | 1.6 Hz), 7.169 (1H, dd, J = 9.1, 9.1 Hz), | |
| | | 7.034 (1H, m), 3.454 (3H, s), 3.267 (3H, | |
| | | s). | |
| 89 | 4-(3-Chloro-4-fluoro-phenyl)-1,2- | ¹H NMR (300 MHz, DMSO-d6): | 358.22 |
| | dimethyl-5-[1,2,4]triazolo[1,5- | 9.200 (1H, m), 8.626 (1H, s), 7.974 (1H, d, | |
| | a]pyridin-6-yl-1,2-dihydro- | J = 9.2 Hz), 7.707 (1H, dd, J = 7.6, 2.2 Hz), | |
| | pyrazol-3-one | 7.543 (1H, dd, J = 9.2, 1.7 Hz), 7.197 (1H, | |
| | | dd, J = 9.0, 9.0 Hz), 7.077 (1H, m), | |
| | | 3.441 (3H, s), 3.289 (3H, s). | |
| 90 | 4-m-Tolyl-5-[1,2,4]triazolo[1,5- | ¹H NMR (300 MHz, DMSO-d6): | 292.23 |
| | a]pyridin-6-yl-1,2-dihydro- | 8.891 (1H, m), 8.520 (1H, s), 7.822 (1H, d, | |
| | pyrazol-3-one | J = 9.4 Hz), 7.469 (1H, d, J = 9.2 Hz), | |
| | | 7.209-7.141 (2H, m), 7.026 (1H, d, J = 7.5 Hz), | |
| | | 2.243 (3H, s). | |
| 91 | 2-Phenyl-4-m-tolyl-5- | ¹H NMR (300 MHz, DMSO-d6): | 368.23 |
| | [1,2,4]triazolo[1,5-a]pyridin-6-yl- | 11.136 (1H, s (br)), 8.718 (1H, s (br)), 8.480 (1H, | |
| | 1,2-dihydro-pyrazol-3-one | s), 7.880-7.777 (3H, m), 7.691 (1H, d, | |
| | | J = 9.7 Hz), 7.530 (2H, t, J = 7.9 Hz), | |
| | | 7.355 (1H, t, J = 7.5 Hz), 7.285 (1H, t, J = 7.5 Hz), | |
| | | 7.244 (1H, s (br)), 7.155 (1H, d, J = 7.5 Hz), | |
| | | 7.107 (1H, d, J = 7.9 Hz), 2.310 (3H, s). | |
| 92 | 4-(5-Oxo-4-m-tolyl-3- | ¹H NMR (300 MHz, DMSO-d6): | 447.15 |
| | [1,2,4]triazolo[1,5-a]pyridin-6-yl- | 8.761 (1H, m), 8.498 (1H, s), 8.097 (2H, d, | |
| | 2,5-dihydro-pyrazol-1-yl)- | J = 8.9 Hz), 7.959 (2H, d, J = 8.9 Hz), | |
| | benzenesulfonamide | 7.834 (1H, d, J = 9.2 Hz), 7.689 (1H, d, J = 9.2 Hz), | |
| | | 7.420 (2H, s), 7.329-7.231 (2H, m), | |
| | | 7.171 (1H, d, J = 7.8 Hz), 7.115 (1H, d, J = 7.5 Hz), | |
| | | 2.313 (3H, s). | |

### VI. Assays

The TGFβ inhibitory activity of compounds of formula (I) can be assessed by methods described in the following examples.

### Example 93

### Fluorescence Polarization Assay for Evaluating Inhibition of TGFβ Receptor

Competitive displacement using a fluorescence polarization assay utilized an Oregon green-labeled ALK4/5 inhibitor, which was shown to bind with high affinity to ALK5 (Kd, 0.34+0.01 nmol/L) and ALK4 (Kd, 0.53+0.03 nmol/L), using fluorescence polarization saturation curve analysis. Varying concentrations of compounds of formula (I) and 25 nmol/L of the Oregon Green-labeled ALK4/5 inhibitor were incubated (1 hour, room temperature, in the dark) with 4.5 mol/L of hALK4-K or hALK5-K, 30 mmol/L Hepes pH 7.5, 20 mmol/L NaCl, 1 mmol/L MgCl2, 100 mmol/L KCl, 0.01% BSA, 0.01% Tween-20 at a final concentration of 1% DMSO in black 96-well Microfluor 2 plates (Cat. No. 7205, ThermoLab Systems).

The signal was detected at excitation/emission settings of 490/530 nanometers using an Analyst HT (LJL BioSystems, Sunnyvale, Calif.). The IC₅₀ values for the tested compounds of formula (I) were determined by nonlinear regression and their Kᵢ values were calculated from the Cheng-Prusoff equation.

### Example 94

### Cell-Free Assay for Evaluating Inhibition of Autophosphorylation of TGFβ Type I Receptor

The serine-threonine kinase activity of TGFβ type I receptor was measured as the autophosphorylation activity of the cytoplasmic domain of the receptor containing an N-terminal poly histidine, TEV cleavage site-tag, e.g., His-TGFβRI. The His-tagged receptor cytoplasmic kinase domains were purified from infected insect cell cultures using the Gibco-BRL FastBac HTh baculovirus expression system.

To a 96-well Nickel FlashPlate (NEN Life Science, Perkin Elmer) was added 20 μl of 1.25 μCi ³³P-ATP/25 μM ATP in assay buffer (50 mM Hepes, 60 mM NaCl, 1 mM MgCl₂, 2 mM DTT, 5 mM MnCl₂, 2% glycerol, and 0.015% Brij® 35). 10 μl of each test compound of formula (I) prepared in 5% DMSO solution were added to the FlashPlate. The assay was then initiated with the addition of 20 ul of assay buffer containing 12.5 μmol of His-TGFβRI to each well. Plates were incubated for 30 minutes at room temperature and the reactions were then terminated by a single rinse with TBS. Radiation from each well of the plates was read on a TopCount (Packard). Total binding (no inhibition) was defined as counts measured in the presence of DMSO solution containing no test compound and non-specific binding was defined as counts measured in the presence of EDTA or no-kinase control.

Alternatively, the reaction performed using the above reagents and incubation conditions but in a microcentrifuge tube was analyzed by separation on a 4-20% SDS-PAGE gel and the incorporation of radiolabel into the 40 kDa His-TGFβRI SDS-PAGE band was quantitated on a Storm Phosphoimager (Molecular Dynamics).

Compounds of formula (I) typically exhibited IC₅₀ values of less than 10 μM; some exhibited IC₅₀ values of less than 1 μM; and some even exhibited IC₅₀ values of less than 50 nM.

### Example 95

### Cell-Free Assay for Evaluating Inhibition of Activin Type I Receptor Kinase Activity

Inhibition of the Activin type I receptor (Alk 4) kinase autophosphorylation activity by tested compounds of formula (I) can be determined in a similar manner to that described above in Example 85 except that a similarly His-tagged form of Alk4 (His-Alk 4) is used in place of the His-TGFβRI.

### Example 96

### TGFβ Type I Receptor Ligand Displacement FlashPlate Assay

50 nM of tritiated 4-(3-pyridin-2-yl-1H-pyrazol-4-yl)-quinoline (custom-ordered from PerkinElmer Life Science, Inc., Boston, Mass.) in assay buffer (50 mM Hepes, 60 mM NaCl₂, 1 mM MgCl₂, 5 mM MnCl₂, 2 mM 1,4-dithiothreitol (DTT), 2% Brij® 35; pH 7.5) was premixed with a test compound of formula (I) in 1% DMSO solution in a v-bottom plate. Control wells containing either DMSO without any test compound or control compound in DMSO were used. To initiate the assay, His-TGFβ Type I receptor in the same assay buffer (Hepes, NaCl₂, MgCl₂, MnCl₂, DTT, and 30% Brij® added fresh) was added to a nickel coated FlashPlate (PE, NEN catalog number: SMP 107), while the control wells contained only buffer (i.e., no His-TGFβ Type I receptor). The premixed solution of tritiated 4-(3-pyridin-2-yl-1H-pyrazol-4-yl)-quinoline and test compound of formula (I) was then added to the wells. The wells were aspirated after an hour at room temperature and radioactivity in wells (emitted from the tritiated compound) was measured using TopCount (PerkinElmer, Boston).

Compounds of formula (I) typically exhibited Kᵢ values of less than 10 μM; some exhibited Kᵢ values of less than 1 μM; and some even exhibited Kᵢ values of less than 50 nM.

### Example 97

### Assay for Evaluating Cellular Inhibition of TGFβ Signaling and Cytotoxicity

Biological activity of the compounds of formula (I) was determined by measuring their ability to inhibit TGFβ-induced PAI-Luciferase reporter activity in HepG2 cells.

HepG2 cells were stably transfected with the PAI-luciferase reporter grown in DMEM medium containing 10% FBS, penicillin (100 U/ml), streptomycin (100 μg/ml), L-glutamine (2 mM), sodium pyruvate (1 mM), and non-essential amino acids (1×). The transfected cells were then plated at a concentration of 2.5×10⁴ cells/well in 96 well plates and starved for 3-6 hours in media with 0.5% FBS at 37° C. in a 5% CO₂ incubator. The cells were then stimulated with 2.5 ng/ml TGFβ ligand in the starvation media containing 1% DMSO either in the presence or absence of a test compound of formula (I) and incubated as described above for 24 hours. The media was washed out the following day and the luciferase reporter activity was detected using the LucLite Luciferase Reporter Gene Assay kit (Packard, cat. no. 6016911) as recommended. The plates were read on a Wallac Microbeta plate reader, the reading of which was used to determine the IC₅₀ values of compounds of formula (I) for inhibiting TGFβ-induced PAI-Luciferase reporter activity in HepG2 cells. Compounds of formula (I) typically exhibited IC₅₀ values of less 10 uM.

Cytotoxicity was determined using the same cell culture conditions as described above. Specifically, cell viability was determined after overnight incubation with the CytoLite cell viability kit (Packard, cat. no. 6016901). Compounds of formula (I) typically exhibited LD₂₅ values greater than 10 μM.

### Example 98

### Assay for Evaluating Inhibition of TGFβ Type I Receptor Kinase Activity in Cells

The cellular inhibition of activin signaling activity by the test compounds of formula (I) can be determined in a similar manner as described above in Example 88 except that 100 ng/ml of activin can be added to serum starved cells in place of the 2.5 ng/ml TGFβ.

### Example 99

### Assay for TGFβ-Induced Collagen Expression

Step A: Preparation of Immortalized Collagen Promoter-Green Fluorescent Protein Cells

Fibroblasts are derived from the skin of adult transgenic mice expressing Green Fluorescent Protein (GFP) under the control of the collagen 1A1 promoter (see Krempen, K. et al., Gene Exp., 8: 151-163 (1999)). Cells are immortalized with a temperature sensitive large T antigen that is in an active stage at 33° C. Cells are expanded at 33° C. and then transferred to 37° C. at which temperature the large T antigen becomes inactive (see Xu, S. et al., Exp. Cell Res., 220: 407-414 (1995)). Over the course of about 4 days and one split, the cells cease proliferating. Cells are then frozen in aliquots sufficient for a single 96 well plate.

Step B: Assay of TGFβ-Induced Collagen-GFP Expression

Cells are thawed, plated in complete DMEM (contains non-essential amino acids, 1 mM sodium pyruvate and 2 mM L-glutamine) with 10% fetal calf serum, and then incubated for overnight at 37° C., 5% CO₂. The cells are trypsinized in the following day and transferred into 96 well format with 30,000 cells per well in 50 μl complete DMEM containing 2% fetal calf serum, but without phenol red. The cells are incubated at 37° C. for 3 to 4 hours to allow them to adhere to the plate. Solutions containing a test compound of formula (I) are then added to wells with no TGFβ (in triplicates), as well as wells with 1 ng/ml TGFβ (in triplicates). DMSO is also added to all of the wells at a final concentration of 0.1%. GFP fluorescence emission at 530 nm following excitation at 485 nm is measured at 48 hours after the addition of solutions containing a test compound on a CytoFluor microplate reader (PerSeptive Biosystems). The data are then expressed as the ratio of TGFβ-induced to non-induced for each test sample.

### Example 100

### Assay for Evaluating Inhibition and/or Prevention of Restinosis (Stenotic Fibrotic Response Balloon Catheter Injury of the Rat Carotid Artery)

The ability of compounds of formula (I) to prevent the stenotic fibrotic response is tested by administration of the test compounds to rats that have undergone balloon catheter injury of the carotid artery. The test compounds are administered intravenously, subcutaneously or orally.

Sprague Dawley rats (400 g, 3 to 4 months old) are anesthetized by inter paratenal i.p. injection with 2.2 mg/kg xylazine (AnaSed, Lloyd laboratories) and 50 mg/kg ketamine (Ketalar, Parke-Davis). The left carotid artery and the aorta are denuded with a 2F balloon catheter according to the procedure described in Clowes et al., Lab Invest., 49: 327-333 (1983). Test compounds of formula (I) are each administered to the treatment group (n=5-10 rats) (i.v., p.o., or s.c.; qod, once per day, bid, tid or by continuous s.c. infusion via an Alzet minipump) starting the day of surgery and subsequently for 14 more days. The control group (n=5 rats) receives the same volume of vehicle administered using the same regimen as the test compound-treated rats. The animals are sacrificed under anesthesia 14 days post-balloon injury. Perfusion fixation is carried out under physiological pressure with phosphate buffered (0.1 mol/L, pH 7.4) 4% paraformadehyde. The injured carotid artery is excised, post-fixed and embedded for histological and morphometic analysis. Sections (5 μm) are cut from the proximal, middle and distal segments of the denuded vessel and analyzed using image analysis software. The circumference of the lumen and the lengths of the internal elastic lamina (IEL) and the external elastic lamina (EEL) are determined by tracing along the luminal surface the perimeter of the neointima (IEL) and the perimeter of the tunica media (EEL) respectively. The lumen (area within the lumen), medial (area between the IEL and EEL) and intimal (area between the lumen and the IEL) areas are also determined using morphometric analysis. Statistical analysis is used ANOVA to determine statistically significant differences between the means of treatment groups (p≦0.05). Multiple comparisons between groups is then performed using the Scheffe test. The Student t test is used to compare the means between 2 groups, and differences are considered significant if P≦0.05. All data are shown as mean±SEM.

The TGFB inhibition activities of several compounds of the present invention were assayed according to the examples above. Some assayed compounds exhibited an IC₅₀ of less than 10 μM (e.g., less than 5.0 μM, 4.5 μM, 4.0 μM, 3.5 μM or 2.5 μM).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A compound of the following formula (I): 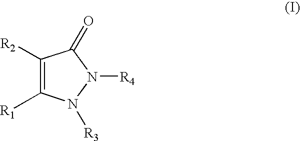
or an N-oxide or a pharmaceutically acceptable salt thereof wherein,
R₁ is an 8-12 membered saturated, partially unsaturated, or fully unsaturated bicyclic ring system having 0-5 heteroatoms independently selected from O, S, or N, in which, R₁ is optionally substituted with up to 5 substituents selected from (Y—R₅);
Each of R₂ and R₃ is independently hydrogen, halo, aliphatic, cycloaliphatic, (cycloaliphatic)alkyl, aryl, araliphatic, heterocycloaliphatic, (heterocycloaliphatic)alkyl, heteroaryl, or heteroaraliphatic;
R₄ is hydrogen, halo, aliphatic, cycloaliphatic, (cycloaliphatic)alkyl, aryl, araliphatic, heterocycloaliphatic, (heterocycloaliphatic)alkyl, heteroaryl, or heteroaraliphatic, each of which is optionally substituted with 1 to 3 of (Y—R₅),
or, R₃ and R₄, together with the nitrogen atoms to which they are attached, form a 5- to 7-membered heterocycloaliphatic ring optionally substituted with 1 to 3 of (Y—R₅);
Each R₅ is independently hydrogen, halo, an aliphatic, an cycloaliphatic, an (cycloaliphatic)alkyl, an aryl, an araliphatic, an heterocycloaliphatic, an (heterocycloaliphatic)alkyl, or an heteroaryl;
Each Y is independently a bond, —C(O)—, —C(O)—O—, —O—C(O)—, —S(O)ₚ—O—, —O—S(O)ₚ—, —C(O)—N(R^{b})—, —N(R^{b})—C(O)—, —O—C(O)—N(R^{b})—, —N(R^{b})—C(O)—O—, —O—S(O)ₚ—N(R^{b})—, —N(R^{b})—S(O)ₚ—O—, —N(R^{b})—C(O)—N(R^{c})—, —N(R^{b})—S(O)ₚ—N(R^{c})—, —C(O)—N(R^{b})—S(O)ₚ—, —S(O)ₚ—N(R^{b})—C(O)—, —C(O)—N(R^{b})—S(O)ₚ—N(R^{c})—, —C(O)—O—S(O)ₚ—N(R^{b})—, —N(R^{b})—S(O)ₚ—N(R^{c})—C(O)—, —N(R^{b})—S(O)ₚ—O—C(O)—, —S(O)ₚ—N(R^{b}), —N(R^{b})—S(O)ₚ—, —N(R^{b})—, —S(O)ₚ—, —O—, —S—, or —(C(R^{b})(R^{c}))_{q}—;
Each of R^{b} and R^{c} is independently hydrogen, hydroxy, alkyl, alkoxy, amino, aryl, aralkyl, heterocycloalkyl, heteroaryl, or heteroaralkyl;
p is 1 or 2, and
q is 1-4;
provided that if R₁ is a benzimidazol-6-yl, the nitrogen atom at the first position of the benzimidazole ring is not directly substituted with sulfonyl.

2. The compound of claim 1, wherein R₁ is a 9 to 11 membered bicyclic ring system.

3. The compound of claim 2, wherein R₁ is an aromatic 9- or 10-membered bicyclic ring system.

4. The compound of claim 3, wherein R₁ is a bicyclic heteroaryl.

5. The compound of claim 4, wherein R₁ is an phenyl fused with a 4- to 8-membered monocyclic heterocycloaliphatic or heteroaryl.

6. The compound of claim 5, wherein R₁ is an indolizinyl, indolyl, isoindolyl, 3H-indolyl, indolinyl, benzo[b]furyl, benzo[b]thiophenyl, quinolinyl, or isoquinolinyl.

7. The compound of claim 6, wherein R₁ is substituted with halo, aliphatic, cycloaliphatic, heterocycloaliphatic, (cycloaliphatic)aliphatic, (heterocycloaliphatic)aliphatic, alkoxy, amino, acyl, carboxy, amido, sulfonyl, sulfamoyl, sulfanyl, sulfinyl, aryl, heteroaryl, heteroaralkyl, or aralkyl.

8. The compound of claim 5, wherein R₁ is a phenyl fused with a 4- to 8-membered monocyclic heterocycle in which the heterocycle includes 2 or more heteroatoms.

9. The compound of claim 8, wherein R₁ is a 1H-indazolyl, benzimidazolyl, benzthiazolyl, cinnolyl, phthalazyl, quinazolyl, quinoxalyl, or 1,8-naphthyridyl.

10. The compound of claim 9, wherein R₁ is a 1H-indazolyl, benzthiazolyl, cinnolyl, phthalazyl, quinazolyl, quinoxalyl, or 1,8-naphthyridyl.

11. The compound of claim 9, wherein R₁ is substituted with aliphatic, cycloaliphatic, heterocycloaliphatic, (cycloaliphatic)aliphatic, (heterocycloaliphatic)aliphatic, amino, amido, sulfamoyl, carboxy, sulfonyl, alkoxy, sulfanyl, sulfinyl, aryl, heteroaryl, heteroaralkyl, or aralkyl.

12. The compound of claim 5, wherein R₁ is phenyl fused with a 4- to 8-membered monocyclic heterocycle in which the heterocycle includes three heteroatoms.

13. The compound of claim 12, wherein R₁ is benzo-1,2,5-thiadiazolyl.

14. The compound of claim 1, wherein R₁ is quinoxal-1-yl, quinoxal-2-yl, quinoxal-7-yl, or quinoxal-8-yl, cinnol-1-yl, cinnol-2-yl, cinnol-3-yl, cinnol-4-yl, cinnol-5-yl, cinnol-6-yl, cinnol-7-yl, cinnol-8-yl, phthalaz-1-yl, phthalaz-2-yl, phthalaz-3-yl, phthalaz-4-yl, phthalaz-5-yl, phthalaz-6-yl, phthalaz-7-yl, phthalaz-8-yl, quinazol-1-yl, quinazol-2-yl, quinazol-3-yl, quinazol-4-yl, quinazol-5-yl, quinazol-6-yl, quinazol-7-yl, quinazol-8-yl, 1,8-naphthyrid-1-yl, 1,8-naphthyrid-2-yl, 1,8-naphthyrid-3-yl, 1,8-naphthyrid-4-yl, 1,8-naphthyrid-5-yl, 1,8-naphthyrid-6-yl, 1,8-naphthyrid-7-yl, or 1,8-naphthyrid-8-yl.

15. The compound of claim 1, wherein R₁ is substituted with alkyl, cycloalkyl, heterocycloalkyl, amido, amino, sulfamoyl, sulfonyl, aryl, heteroaryl, cyano, nitro, hydroxyl, heteroaralkyl, or aralkyl.

16. The compound of claim 1, wherein R₂ is aryl or heteroaryl.

17. The compound of claim 16, wherein R₂ is phenyl.

18. The compound of claim 17, wherein R₂ is substituted at the meta position relative to the point of attachment between R₂ and the pyrazalone ring.

19. The compound of claim 18, wherein R₂ is substituted with halo, amido, carboxy, amino, alkoxy, sulfonyl, sulfanyl, sulfinyl, or aliphatic at the meta position relative to the point of attachment between R₂ and the pyrazalone ring.

20. The compound of claim 17, wherein R₂ is substituted at the ortho position relative to the point of attachment between R₂ and the pyrazalone ring.

21. The compound of claim 20, wherein R₂ is substituted with amino, cyanoalkyl, alkoxyalkyl, alkoxy, alkyl, or cyano at the ortho position relative to the point of attachment between R₂ and the pyrazalone ring.

22. The compound of claim 17, wherein R₂ is substituted at the para position relative to the point of attachment between R₂ and the pyrazalone ring.

23. The compound of claim 22, wherein R₂ is substituted with halo, cyanoalkyl, morpholinylsulfonyl, or haloalkyl at the para position relative to the point of attachment between R₂ and the pyrazalone ring.

24. The compound of claim 16, wherein R₂ is furyl, thiophenyl, 2H-pyrrolyl, pyrrolyl, oxazolyl, thiazolyl, imidazolyl, pyridyl, pyridazyl, pyramidyl, pyrazolyl, or pyrazyl.

25. The compound of claim 24, wherein R₂ is substituted with halo, carboxy, amido, alkoxy, sulfamoyl, sulfonyl, aminoalkyl, alkoxyalkyl, alkylcarbonyl, amino, or aliphatic.

26. The compound of claim 16, wherein R₂ is a bicyclic aryl or a bicyclic heteroaryl.

27. The compound of claim 26, wherein R₂ is quinolyl, indolyl, 3H-indolyl, isoindolyl, benzo[b]-4H-pyranyl, cinnolyl, quinoxylyl, benzimidazyl, benzo-1,2,5-thiadiazolyl, benzo-1,2,5-oxadiazolyl, or benzthiophenyl.

28. The compound of claim 27, wherein R₂ is substituted halo, carboxy, alkylcarbonyl, amido, alkoxy, sulfamoyl, sulfonyl, aminoalkyl, alkoxyalkyl, alkylcarbonyl, amino, or aliphatic.

29. The compound of claim 1, wherein R₃ is hydrogen, halo, aliphatic, cycloaliphatic, heterocycloaliphatic, amino, amido, hydroxy, alkoxy, aryl, heteroaryl, sulfonyl, sulfinyl, or sulfanyl.

30. The compound of claim 29, wherein R₃ is aliphatic, cycloaliphatic, heterocycloaliphatic, amino, amido, alkoxy, aryl, or heteroaryl, each optionally substituted with 1-3 substituents independently selected from hydrogen, halo, aliphatic, cycloaliphatic, heterocycloaliphatic, aryl, heteroaryl, hydroxy, alkoxy, amino, cyano, carboxy, carbonyl, sulfonyl, sulfanyl, and sulfinyl.

31. The compound of claim 30, wherein R₃ is alkyl, aryl, or heteroaryl.

32. The compound of claim 31, wherein R₃ is furyl, thiopheny, 2H-pyrrolyl, pyrrolyl, oxazolyl, thiazolyl, imidazolyl, pyrazolyl, pyridyl, pyridazyl, pyrimidyl, pyrazyl, or 1,3,5-triazyl.

33. The compound of claim 1, wherein R₄ is hydrogen, halo, aliphatic, cycloaliphatic, heterocycloaliphatic, amino, amido, hydroxide, alkoxy, aryl, heteroaryl, sulfonyl, sulfinyl, or sulfanyl.

34. The compound of claim 33, wherein R₄ is aliphatic, cycloaliphatic, heterocycloaliphatic, amino, amido, alkoxy, aryl, or heteroaryl, each optionally substituted with 1-3 substituents independently selected from hydrogen, halo, aliphatic, cycloaliphatic, heterocycloaliphatic, aryl, heteroaryl, hydroxyl, alkoxy, sulfonyl, sulfanyl, or sulfinyl.

35. The compound of claim 34, wherein R₄ is alkyl.

36. The compound of claim 35, wherein R₄ is haloalkyl.

37. The compound of claim 35, wherein the alkyl is optionally substituted with cycloaliphatic.

38. The compound of claim 35, wherein the alkyl is optionally substituted with bicycloaliphatic.

39. The compound of claim 35, wherein the alkyl is optionally substituted with aryl.

40. The compound of claim 33, wherein R₄ is heteroaryl.

41. The compound of claim 40, wherein R₄ is furyl, thiopheny, 2H-pyrrolyl, pyrrolyl, oxazolyl, thiazolyl, imidazolyl, pyrazolyl, pyridyl, pyridazyl, pyrimidyl, pyrazyl, or 1,3,5-triazyl.

42. The compound of claim 1, wherein R₃ and R₄ together with the nitrogen atoms to which they are attached form a 5 or 6 membered ring optionally substituted with 1-3 substituents independently selected from hydrogen, halo, aliphatic, cycloaliphatic, heterocycloaliphatic, aryl, heteroaryl, hydroxyl, alkoxy, sulfonyl, sulfanyl, and sulfinyl.

43. The compound of claim 1, wherein R₁ is

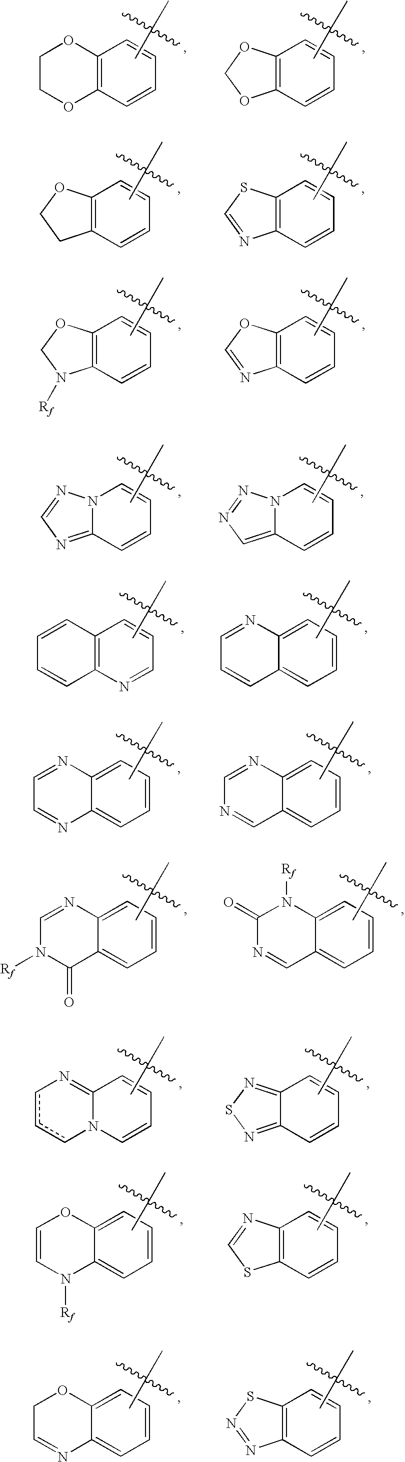

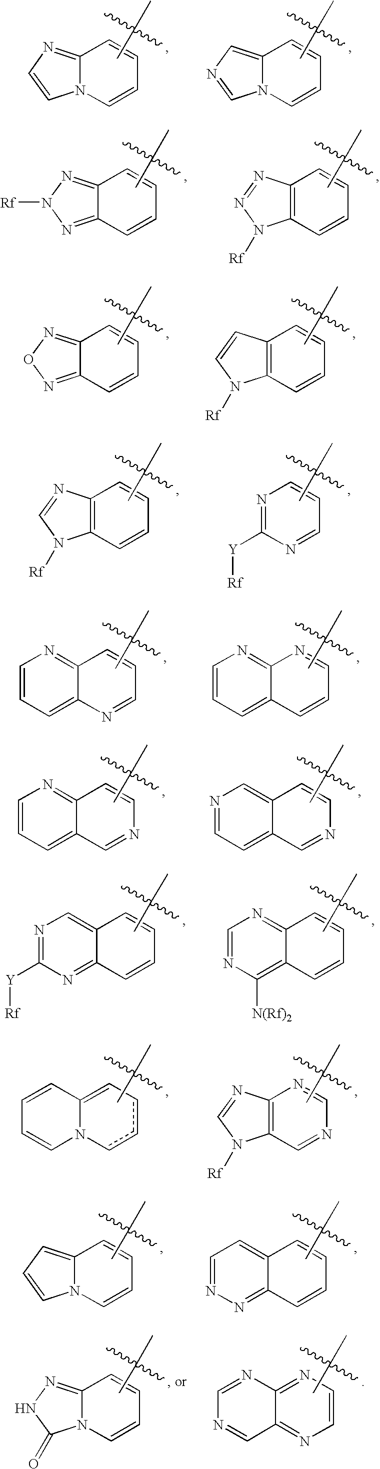

44. The compound of claim 1, wherein R₂ is

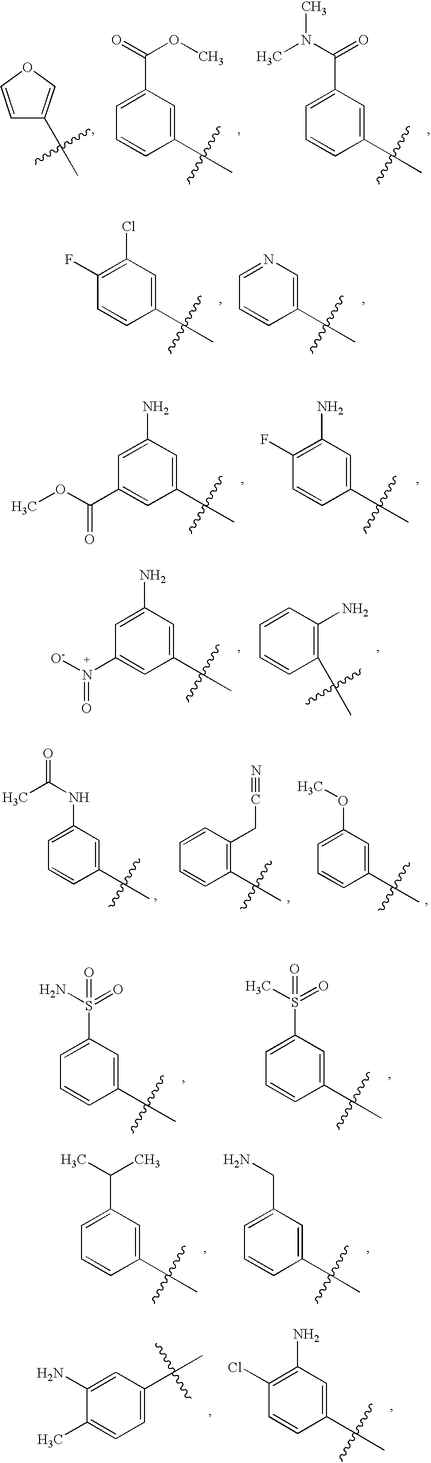

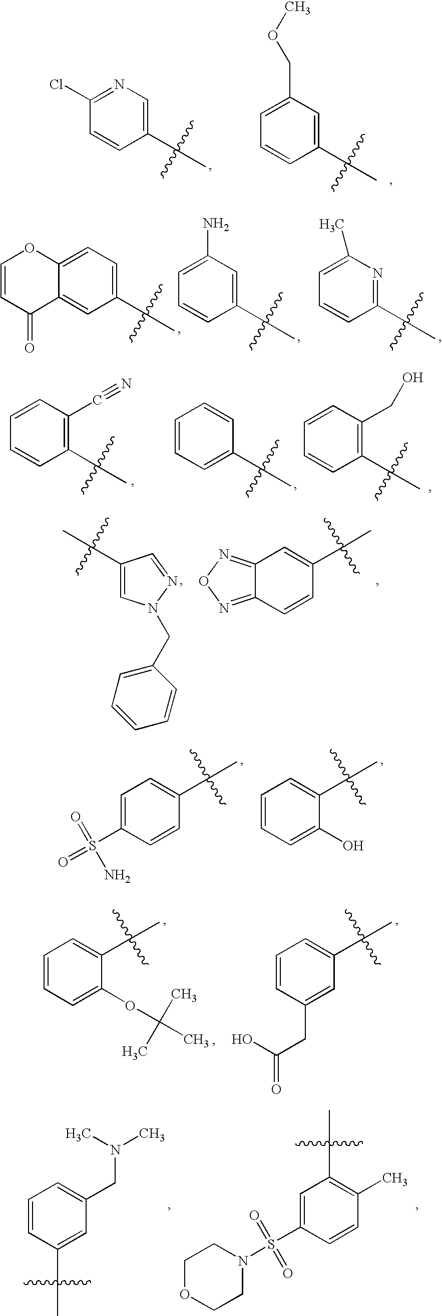

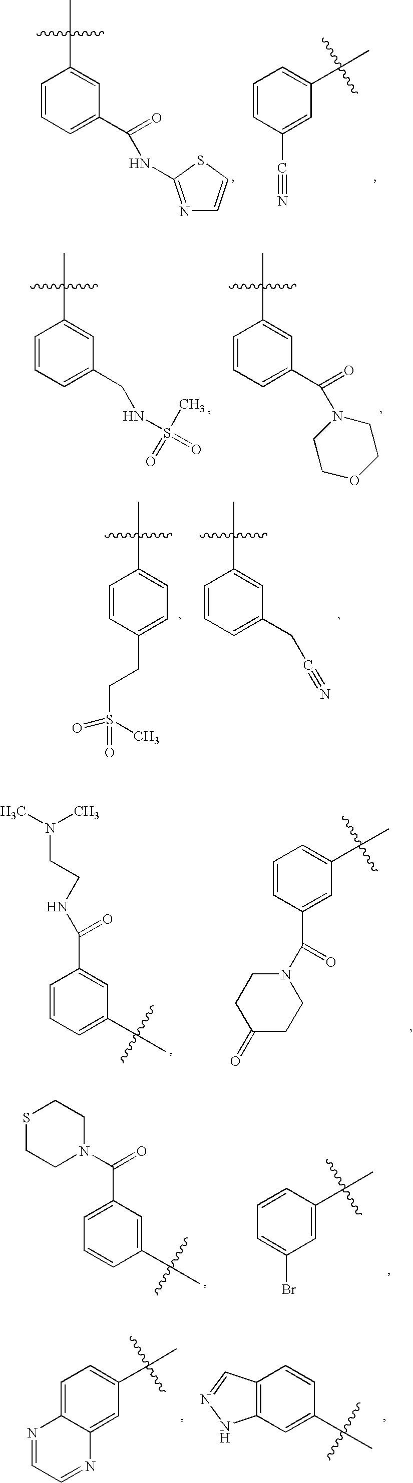

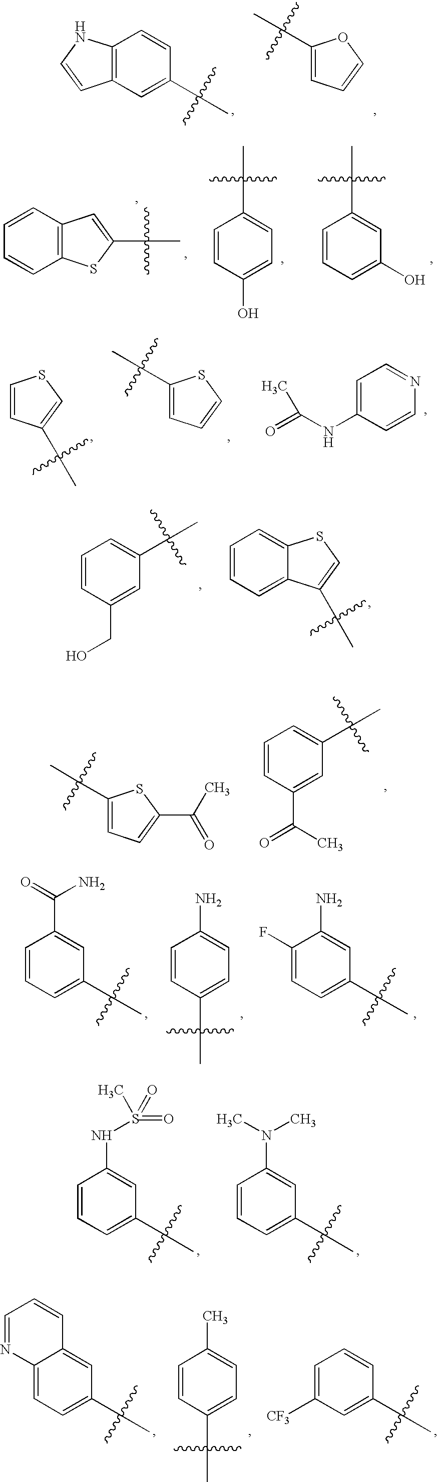

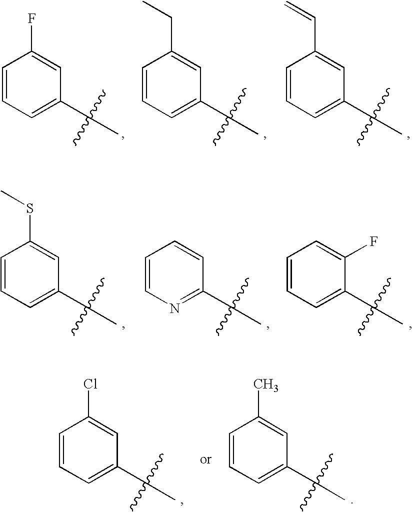

45. A compound selected from the group consisting of
2-(1,2-dimethyl-3-oxo-5-quinoxalin-6-yl-2,3-dihydro-1H-pyrazol-4-yl)-benzonitrile;
1,2-dimethyl-5-quinoxalin-6-yl-4-thiophen-3-yl-1,2-dihydro-pyrazol-3-one;
5-benzo[1,2,5]thiadiazol-5-yl-1,2-diethyl-4-m-tolyl-1,2-dihydro-pyrazol-3-one;
4-(2-methyl-5-oxo-3-quinoxalin-6-yl-4-m-tolyl-2,5-dihydro-pyrazol-1-ylmethyl)-benzoic acid methyl ester;
1-methyl-5-quinoxalin-6-yl-4-m-tolyl-2-(4-trifluoromethoxy-benzyl)-1,2-dihydro-pyrazol-3-one;
1-methyl-5-quinoxalin-6-yl-2-(4-trifluoromethyl-phenyl)-1,2-dihydro-pyrazol-3-one;
1,2-dimethyl-4-pyridin-2-yl-5-quinoxalin-6-yl-1,2-dihydro-pyrazol-3-one;
2-pyridin-2-yl-3-quinoxalin-6-yl-6,7-dihydro-5H-pyrazolo[1,2-a]pyrazol-1-one;
2-pyridin-2-yl-3-quinoxalin-6-yl-5,6,7,8-tetrahydro-pyrazolo[1,2-a]pyridazin-1-one;
1,2-dimethyl-5-quinoxalin-6-yl-4-m-tolyl-1,2-dihydro-pyrazol-3-one;
4-(3-chloro-phenyl)-1,2-dimethyl-5-quinoxalin-6-yl-1,2-dihydro-pyrazol-3-one;
4-(2-fluoro-phenyl)-1,2-dimethyl-5-quinoxalin-6-yl-1,2-dihydro-pyrazol-3-one;
1,2-diethyl-4-pyridin-2-yl-5-quinoxalin-6-yl-1,2-dihydro-pyrazol-3-one;
1,2-dimethyl-4-pyridin-2-yl-5-quinoxalin-6-yl-1,2-dihydro-pyrazol-3-one;
4-(3-fluoro-phenyl)-1,2-dimethyl-5-quinoxalin-6-yl-1,2-dihydro-pyrazol-3-one;
1,2-dimethyl-5-quinoxalin-6-yl-4-(3-trifluoromethyl-phenyl)-1,2-dihydro-pyrazol-3-one;
4-(3-amino-4-fluoro-phenyl)-1,2-dimethyl-5-quinoxalin-6-yl-1,2-dihydro-pyrazol-3-one;
1,2-dimethyl-4-quinolin-6-yl-5-quinoxalin-6-yl-1,2-dihydro-pyrazol-3-one;
4-(3-dimethylamino-phenyl)-1,2-dimethyl-5-quinoxalin-6-yl-1,2-dihydro-pyrazol-3-one;
3-(1,2-dimethyl-3-oxo-5-quinoxalin-6-yl-2,3-dihydro-1H-pyrazol-4-yl)-benzene sulfonamide;
4-(4-amino-phenyl)-1,2-dimethyl-5-quinoxalin-6-yl-1,2-dihydro-pyrazol-3-one;
3-(1,2-dimethyl-3-oxo-5-quinoxalin-6-yl-2,3-dihydro-1H-pyrazol-4-yl)-benzamide;
1,2-dimethyl-5-quinoxalin-6-yl-4-thiophen-2-yl-1,2-dihydro-pyrazol-3-one;
4-(3-acetyl-phenyl)-1,2-dimethyl-5-quinoxalin-6-yl-1,2-dihydro-pyrazol-3-one;
4-(5-acetyl-thiophen-2-yl)-1,2-dimethyl-5-quinoxalin-6-yl-1,2-dihydro-pyrazol-3-one;
4-benzo[b]thiophen-3-yl-1,2-dimethyl-5-quinoxalin-6-yl-1,2-dihydro-pyrazol-3-one;
4-(3-hydroxymethyl-phenyl)-1,2-dimethyl-5-quinoxalin-6-yl-1,2-dihydro-pyrazol-3-one;
3-(1,2-dimethyl-3-oxo-5-quinoxalin-6-yl-2,3-dihydro-1H-pyrazol-4-yl)-benzonitrile;
N-[4-(1,2-dimethyl-3-oxo-5-quinoxalin-6-yl-2,3-dihydro-1H-pyrazol-4-yl)-phenyl]-acetamide;
4-(3-hydroxy-phenyl)-1,2-dimethyl-5-quinoxalin-6-yl-1,2-dihydro-pyrazol-3-one;
4-(4-hydroxy-phenyl)-1,2-dimethyl-5-quinoxalin-6-yl-1,2-dihydro-pyrazol-3-one;
4-furan-2-yl-1,2-dimethyl-5-quinoxalin-6-yl-1,2-dihydro-pyrazol-3-one;
4-(3-bromo-phenyl)-1,2-dimethyl-5-quinoxalin-6-yl-1,2-dihydro-pyrazol-3-one;
4-benzo[b]thiophen-2-yl-1,2-dimethyl-5-quinoxalin-6-yl-1,2-dihydro-pyrazol-3-one;
4-(1H-indol-5-yl)-1,2-dimethyl-5-quinoxalin-6-yl-1,2-dihydro-pyrazol-3-one;
4-(1H-indazol-6-yl)-1,2-dimethyl-5-quinoxalin-6-yl-1,2-dihydro-pyrazol-3-one;
1,2-dimethyl-4,5-di-quinoxalin-6-yl-1,2-dihydro-pyrazol-3-one;
1-[3-(1,2-dimethyl-3-oxo-5-quinoxalin-6-yl-2,3-dihydro-1H-pyrazol-4-yl)-benzoyl]-piperidin-4-one;
1,2-dimethyl-5-quinoxalin-6-yl-4-[3-(thiomorpholine-4-carbonyl)-phenyl]-1,2-dihydro-pyrazol-3-one;
N-(2-dimethylamino-ethyl)-3-(1,2-dimethyl-3-oxo-5-quinoxalin-6-yl-2,3-dihydro-1H-pyrazol-4-yl)-benzamide;
[3-(1,2-dimethyl-3-oxo-5-quinoxalin-6-yl-2,3-dihydro-1H-pyrazol-4-yl)-phenyl]-acetonitrile;
N-[4-(1,2-dimethyl-3-oxo-5-quinoxalin-6-yl-2,3-dihydro-1H-pyrazol-4-yl)-benzyl]-methanesulfonamide;
1,2-dimethyl-4-[3-(morpholine-4-carbonyl)-phenyl]-5-quinoxalin-6-yl-1,2-dihydro-pyrazol-3-one;
N-[3-(1,2-dimethyl-3-oxo-5-quinoxalin-6-yl-2,3-dihydro-1H-pyrazol-4-yl)-benzyl]-methanesulfonamide;
3-(1,2-dimethyl-3-oxo-5-quinoxalin-6-yl-2,3-dihydro-1H-pyrazol-4-yl)-N-thiazol-2-yl-benzamide;
1,2-dimethyl-4-[2-methyl-5-(morpholine-4-sulfonyl)-phenyl]-5-quinoxalin-6-yl-1,2-dihydro-pyrazol-3-one;
4-(3-dimethylaminomethyl-phenyl)-1,2-dimethyl-5-quinoxalin-6-yl-1,2-dihydro-pyrazol-3-one;
[3-(1,2-dimethyl-3-oxo-5-quinoxalin-6-yl-2,3-dihydro-1H-pyrazol-4-yl)-phenyl]-acetic acid;
4-(2-tert-butoxymethyl-phenyl)-1,2-dimethyl-5-quinoxalin-6-yl-1,2-dihydro-pyrazol-3-one;
4-(2-hydroxy-phenyl)-1,2-dimethyl-5-quinoxalin-6-yl-1,2-dihydro-pyrazol-3-one;
4-(1,2-dimethyl-3-oxo-5-quinoxalin-6-yl-2,3-dihydro-1H-pyrazol-4-yl)-benzenesulfonamide;
4-benzo[1,2,5]oxadiazol-5-yl-1,2-dimethyl-5-quinoxalin-6-yl-1,2-dihydro-pyrazol-3-one;
1′-benzyl-1,2-dimethyl-5-quinoxalin-6-yl-1,2-dihydro-1′H-[4,4′]bipyrazolyl-3-one;
4-(3-methoxymethyl-phenyl)-1,2-dimethyl-5-quinoxalin-6-yl-1,2-dihydro-pyrazol-3-one;
4-(2-hydroxymethyl-phenyl)-1,2-dimethyl-5-quinoxalin-6-yl-1,2-dihydro-pyrazol-3-one;
4-(3-benzo[1,2,5]thiadiazol-5-yl-5-methoxy-pyrazol-1-yl)-benzoic acid methyl ester;
1,2-dimethyl-4-phenyl-5-quinoxalin-6-yl-1,2-dihydro-pyrazol-3-one;
1,2-dimethyl-4-(6-methyl-pyridin-2-yl)-5-quinoxalin-6-yl-1,2-dihydro-pyrazol-3-one;
4-(3-aminophenyl)-1,2-dihydro-1,2-dimethyl-5-(quinoxalin-7-yl)pyrazol-3-one,
1,2-dihydro-1,2-dimethyl-4-(4-oxo-4H-chromen-6-yl)-5-(quinoxalin-7-yl)pyrazol-3-one;
4-(6-chloropyridin-3-yl)-1,2-dihydro-1,2-dimethyl-5-(quinoxalin-7-yl)pyrazol-3-one;
4-(3-amino-4-methylphenyl)-1,2-dihydro-1,2-dimethyl-5-(quinoxalin-7-yl)pyrazol-3-one;
4-(3-amino-4-chlorophenyl)-1,2-dihydro-1,2-dimethyl-5-(quinoxalin-7-yl)pyrazol-3-one;
4-(3-(aminomethyl)phenyl)-1,2-dihydro-1,2-dimethyl-5-(quinoxalin-7-yl)pyrazol-3-one;
1,2-dihydro-1,2-dimethyl-4-(3-(methylsulfonyl)phenyl)-5-(quinoxalin-7-yl)pyrazol-3-one;
1,2-dihydro-1,2-dimethyl-4-(3-(aminosulfonyl)phenyl)-5-(quinoxalin-7-yl)pyrazol-3-one;
1,2-dihydro-4-(3-methoxyphenyl)-1,2-dimethyl-5-(quinoxalin-7-yl)pyrazol-3-one;
2-(2-(2,3-dihydro-1,2-dimethyl-3-oxo-5-(quinoxalin-7-yl)-1H-pyrazol-4-yl)phenyl)acetonitrile;
N-(3-(2,3-dihydro-1,2-dimethyl-3-oxo-5-(quinoxalin-7-yl)-1H-pyrazol-4-yl)phenyl)acetamide;
4-(2-aminophenyl)-1,2-dihydro-1,2-dimethyl-5-(quinoxalin-7-yl)pyrazol-3-one;
4-(3-amino-5-nitrophenyl)-1,2-dihydro-1,2-dimethyl-5-(quinoxalin-7-yl)pyrazol-3-one;
1,2-dihydro-1,2-dimethyl-4-(quinolin-8-yl)-5-(quinoxalin-7-yl)pyrazol-3-one;
methyl 3-amino-5-(2,3-dihydro-1,2-dimethyl-3-oxo-5-(quinoxalin-7-yl)-1H-pyrazol-4-yl)benzoate;
1,2-dihydro-1,2-dimethyl-4-(pyridin-3-yl)-5-(quinoxalin-7-yl)pyrazol-3-one;
4-(3-chloro-4-fluorophenyl)-1,2-dihydro-1,2-dimethyl-5-(quinoxalin-7-yl)pyrazol-3-one;
3-(2,3-dihydro-1,2-dimethyl-3-oxo-5-(quinoxalin-7-yl)-1H-pyrazol-4-yl)-N,N-dimethylbenzamide;
methyl 3-(2,3-dihydro-1,2-dimethyl-3-oxo-5-(quinoxalin-7-yl)-1H-pyrazol-4-yl)benzoate;
4-furan-3-yl-1,2-dimethyl-5-quinoxalin-6-yl-1,2-dihydro-pyrazol-3-one;
5-benzo[1,2,5]thiadiazol-5-yl-4-(3-bromo-phenyl)-2-(4-hydroxy-bicyclo[2.2.2]oct-1-ylmethyl)-1-methyl-1,2-dihydro-pyrazol-3-one;
4-(3-ethyl-phenyl)-1,2-dimethyl-5-quinoxalin-6-yl-1,2-dihydro-pyrazol-3-one;
4-(3-isopropyl-phenyl)-1,2-dimethyl-5-quinoxalin-6-yl-1,2-dihydro-pyrazol-3-one;
1,2-dimethyl-4-(3-methylsulfanyl-phenyl)-5-quinoxalin-6-yl-1,2-dihydro-pyrazol-3-one;
1,2-dimethyl-5-quinoxalin-6-yl-4-(3-vinyl-phenyl)-1,2-dihydro-pyrazol-3-one;
1,2-dimethyl-4-(2-methyl-pyridin-4-yl)-5-quinoxalin-6-yl-1,2-dihydro-pyrazol-3-one;
1,2-dimethyl-4-m-tolyl-5-[1,2,4]triazolo[1,5-a]pyridin-6-yl-1,2-dihydro-pyrazol-3-one;
5-benzo[1,2,5]thiadiazol-5-yl-1,2-dimethyl-1,2-dihydro-pyrazol-3-one;
5-benzo[1,2,5]thiadiazol-5-yl-4-bromo-1,2-dimethyl-1,2-dihydro-pyrazol-3-one;
5-benzo[1,2,5]thiadiazol-5-yl-4-(3-chloro-4-fluoro-phenyl)-1,2-dimethyl-1,2-dihydro-pyrazol-3-one;
4-(3-chloro-4-fluoro-phenyl)-1,2-dimethyl-5-[1,2,4]triazolo[1,5-a]pyridin-6-yl-1,2-dihydro-pyrazol-3-one;
4-m-tolyl-5-[1,2,4]triazolo[1,5-a]pyridin-6-yl-1,2-dihydro-pyrazol-3-one;
2-phenyl-4-m-tolyl-5-[1,2,4]triazolo[1,5-a]pyridin-6-yl-1,2-dihydro-pyrazol-3-one; and
4-(5-oxo-4-m-tolyl-3-[1,2,4]triazolo[1,5-a]pyridin-6-yl-2,5-dihydro-pyrazol-1-yl)-benzenesulfonamide.

46. A pharmaceutical composition comprising a compound of claim 1 or 45 and a pharmaceutically acceptable carrier.

47. A method of inhibiting the TGFβ signaling pathway in a subject, comprising administering to said subject an effective amount of a compound of claim 1 or 45.

48. A method of inhibiting the TGFβ type I receptor in a cell, comprising contacting said cell with an effective amount of a compound of claim 1 or 45.

49. A method of reducing the accumulation of excess extracellular matrix induced by TGFβ in a subject, comprising administering to said subject an effective amount of a compound of claim 1 or 45.

50. A method of treating or preventing fibrotic condition in a subject, comprising administering to said subject an effective amount of a compound of claim 1 or 45.

51. The method of claim 50, wherein the fibrotic condition is selected from the group consisting of mesothelioma, acute respiratory distress syndrome (ARDS), atherosclerosis, scleroderma, keloids, glomerulonephritis, diabetic nephropathy, lupus nephritis, hypertension-induced nephropathy, idiopathic pulmonary fibrosis, cholangitis, restenosis, ocular scarring, corneal scarring, hepatic fibrosis, biliary fibrosis, liver cirrhosis, cirrhosis due to fatty liver disease (alcoholic and nonalcoholic steatosis), pulmonary fibrosis, renal fibrosis, sarcoidosis, acute lung injury, drug-induced lung injury, spinal cord injury, CNS scarring, systemic lupus erythematosus, Wegener's granulomatosis, cardiac fibrosis, post-infarction cardiac fibrosis, post-surgical fibrosis, connective tissue disease, radiation-induced fibrosis, chemotherapy-induced fibrosis, transplant arteriopathy, fibrosclerosis, fibrotic cancers, fibroids, fibroma, fibroadenomas, and fibrosarcomas.

52. A method of inhibiting metastasis of tumor cells in a subject, comprising administering to said subject an effective amount of a compound of claim 1 or 45.

53. A method of treating carcinomas mediated by an overexpression of TGFβ, comprising administering to a subject in need of such treatment an effective amount of a compound of claim 1 or 45.

54. The method of claim 53, wherein said carcinomas are selected from the group consisting of carcinomas of the lung, breast, liver, biliary tract, gastrointestinal tract, head and neck, pancreas, prostate, cervix, multiple myeloma, melanoma, glioma, and glioblastomas.

55. A method of treating or preventing restinosis, vascular disease, or hypertension by administering to a subject in need thereof a compound of claim 1 or 45.

56. The method of claim 55, wherein restinosis is coronary restenosis, peripheral restenosis, or carotid restenosis.

57. The method of claim 55, wherein vascular disease is intimal thickening, vascular remodeling, or organ transplant-related vascular disease.

58. The method of claim 57, wherein the vascular disease is intimal thickening or vascular remodeling.

59. The method of claim 55, wherein hypertension is primary or secondary hypertension, systolic hypertension, pulmonary hypertension, or hypertension-induced vascular remodeling.

60. The method of claim 55, wherein the compound is administered locally.

61. The method of claim 55, wherein the compound is administered via an implantable device.

62. The method of claim 61, wherein the device is a delivery pump.

63. The method of claim 61, wherein the device is a stent.
